(19) United States
(12) Patent Application Publication
(10) Pub. No.: US 2023/0228751 A1
(43) Pub. Date: Jul. 20, 2023

(54) APTAMERS AGAINST SARS-COV-2

(71) Applicant: APTAMER DIAGNOSTICS LIMITED, York (GB)

(72) Inventors: Arron TOLLEY, York (GB); David BUNKA, York (GB); Edward BARNES, York (GB); Banushan BALANSETHUPATHY, York (GB); Harriet FLYNN, York (GB); Janice ROBOTTOM, York (GB); Laura MILBURN, York (GB); Phoebe O'HARA, York (GB); Joe REED, York (GB)

(21) Appl. No.: 18/002,224

(22) PCT Filed: Jun. 18, 2021
(86) PCT No.: PCT/GB2021/051559

§ 371 Date: Dec. 16, 2022

(30) Foreign Application Priority Data
Jun. 18, 2020 (GB) 2009351.4
Nov. 4, 2020 (GB) 2017437.1
Apr. 15, 2021 (GB) 2105379.8

(51) Int. Cl. G01N 33/569; G01N 33/543; G01N 33/53

(52) U.S. Cl. CPC G01N 33/56983; G01N 33/54388; G01N 33/5306; G01N 2469/10; G01N 2333/165

(57) ABSTRACT

The invention relates to one or more aptamers isolated against the SARS-CoV-2 spike protein and methods of using the same. Certain embodiments of the invention relate to methods of detecting the presence, absence or amount of SARS-CoV-2 in a sample using the one or more aptamers described herein. In certain embodiments, the invention relates to one or more aptamers that are capable of specifically binding to SARS-CoV-2 proteins, including aptamers that are capable of specifically binding to the S1 subunit (including the receptor binding domain (RBD)) and/or the S2 subunit within their native conformation as part of the SARS-CoV-2 spike protein in its trimeric form or as separate monomers.

### FIELD OF THE INVENTION

Embodiments of the present invention relate to one or more aptamers isolated against the SARS-CoV-2 spike protein and methods of using the same. Certain embodiments of the invention relate to methods of detecting the presence, absence or amount of SARS-CoV-2 in a sample using the one or more aptamers described herein. In certain embodiments, the invention relates to one or more aptamers that are capable of specifically binding to SARS-CoV-2 proteins, including aptamers that are capable of specifically binding to the S1 subunit (including the receptor binding domain (RBD)) and/or the S2 subunit within their native conformation as part of the SARS-CoV-2 spike protein in its trimeric form or as separate monomers.

### BACKGROUND TO THE INVENTION

Severe acute respiratory syndrome coronavirus 2 (SARS-CoV-2), was a previously unknown coronavirus discovered in December 2019 in Wuhan, China. Human-to-human transmission of SARS-CoV-2 is thought to occur primarily via respiratory droplets, with infection a causative agent of the coronavirus disease 2019 (COVID-19). This outbreak of atypical pneumonia was declared a pandemic by the World Health Organization in March 2020.

This atypical pneumonia, which can be fatal, is caused by the replication of SARS-CoV-2 in the lower respiratory tract. The pathophysiology of COVID-19 shows an aggressive inflammatory response, which can result in acute inflammation and pulmonary dysfunction. Therefore, in some instances, disease severity in patients is exacerbated by the host's immune response, with the overproduction of pro-inflammatory cytokines (also known as the cytokine storm) triggering patients to develop acute respiratory distress syndrome.

Population-scale diagnostic testing for COVID-19 is crucial for breaking the chain of SARS-CoV-2 infections. A number of SARS-CoV-2 monoclonal antibodies have been raised against various SARS-CoV-2 antigens, including the proteins that form the viral coat. Such SARS-CoV-2 antibodies are being developed, for example, as point-of-care testing kits. However, such tests may rely on a pair of antibodies (which can be difficult to isolate and expensive to produce). In addition, many of the antibodies which have been isolated to date have specificity issues, leading to false positive results; or sensitivity issues, leading to false negative results.

It is an aim of some embodiments of the present invention to at least partially mitigate some of the problems identified in the prior art by developing detection agents which are more reliable, accurate and/or cheaper to produce as compared to antibody-based tests.

### SUMMARY OF CERTAIN EMBODIMENTS OF THE INVENTION

The present invention relates to the development of one or more aptamers against the SARS-CoV-2 proteins (including the spike protein in its native trimeric form) and methods of using the same.

In certain embodiments, the invention relates to aptamers that are capable of binding to the S1 subunit within the SARS-CoV-2 spike protein (including its native trimeric form). In certain embodiments, the invention relates to aptamers that are capable of binding to the RBD within the S1 subunit.

In certain embodiments, the invention relates to aptamers that are capable of binding to the S2 subunit within the SARS-CoV-2 spike protein (including its native trimeric form).

In certain embodiments, the invention relates to two or more aptamers (e.g., a pair, triplet or quadruplet of aptamers) wherein at least one aptamer binds to the S1 subunit and at least one aptamer binds to a different region of the S1 subunit or the S2 subunit within the SARS-CoV-2 spike protein. Such aptamers are particularly suitable for use in ‘sandwich assay’ formats such as ELISA or lateral flow devices as further described herein.

In certain embodiments, the aptamers of the invention bind to SARS-CoV-2 and also cross-react with other coronaviruses such as SARS-CoV and/or MERS-CoV.

In certain embodiments, the aptamers of the invention specifically bind to SARS-CoV-2 but do not cross-react with other coronaviruses such as SARS-CoV and/or MERS-CoV.

The aptamers described herein are shown to work effectively and provide a simple and quick means of testing the presence, absence or amount of SARS-CoV-2 in a sample using a simple gain-of signal assay format. In particular, the invention provides aptamers that are capable of binding to the S1 subunit (including the RBD) and/or the S2 subunit within the SARS-CoV-2 spike protein (including its native trimeric form) with unexpectedly high affinity and specificity. The aptamers described herein allow viral loads of SARS-CoV-2 to be detected in biological fluids such as saliva.

In certain embodiments, the invention provides one or more aptamers capable of specifically binding to the S1 and/or S2 subunit of the SARS-CoV-2 spike protein wherein the one or more aptamers comprise:
(a) a nucleic acid sequence selected from any one or more of SEQ ID NOs: 4, 8, 9 or 43;
(b) a nucleic acid sequence selected from any one or more of SEQ ID NOs: 134, 140 or 144;
(c) a nucleic acid sequence selected from any one or more of SEQ ID NOs: 146, 150 or 171;
(d) a nucleic acid sequence selected from any one or more of SEQ ID NOs: 177, 179, 183, 188, 190 or 191;
(e) a nucleic acid sequence having at least about 85%, 90%, 95% or 99% identity or more with any one or more of the sequences of (a) to (d); or
(f) a nucleic acid sequence having at least about 15, 20, 25, 30, 35, 40, 45, 50, 55, 60, 65, 70, 75 or more consecutive nucleotides of any one or more of the sequences of (a) to (e).

Advantageously, these aptamers are highly effective in diagnostic assays for the SARS-CoV-2 virus. As described herein, these aptamers exhibit an unexpected wide range of advantageous properties and have also shown to be particularly effective in an ELISA-like assay, lateral flow devices and/or electrochemical sensor formats as compared to other aptamers. For example, these aptamers are capable of unexpectedly high affinity binding to the S1 and/or S2 subunit of the SARS-CoV-2 spike protein (including in its native trimeric form). These aptamers are capable of binding to the SARS-CoV-2 spike protein present in a saliva sample. These aptamers do not cross-react with the homologous spike protein from SARS-CoV and/or MERS-CoV.

In certain embodiments, the one or more aptamers of the invention may comprise:
(a) a nucleic acid sequence selected from any one of SEQ ID NOs 4, 8 or 9; or
(b) a nucleic acid sequence having at least about 85%, 90%, 95% or 99% identity or more with any one or more of the sequences of (a).

Such aptamers are capable of specifically binding to the S1 subunit of the SARS-CoV-2 spike protein. These aptamers exhibit an unexpected wide range of advantageous properties (including binding to SARS-Cov-2 spike protein present in a saliva sample and in its native trimeric form) and have also shown to be particularly effective in ELISA-like formats or lateral flow assays.

In certain embodiments, the one or more aptamers of the invention may comprise:
(a) a nucleic acid sequence selected from any one of SEQ ID NOs 177, 183 or 191; or
(b) a nucleic acid sequence having at least about 85%, 90%, 95% or 99% identity or more with any one or more of the sequences of (a).

Such aptamers are capable of specifically binding to the S2 subunit of the SARS-CoV-2 spike protein.

In certain embodiments, the one or more aptamers of the invention may comprise:
(a) a nucleic acid sequence selected from any one of SEQ ID NOs 179, 188 or 190; or
(b) a nucleic acid sequence having at least about 85%, 90%, 95% or 99% identity or more with any one or more of the sequences of (a).

Such aptamers are unexpectedly capable of binding to the S2 subunit of the SARS-CoV-2 spike protein in its native trimeric form.

In certain embodiments, the invention provides one or more minimal effective fragments of any one or more aptamers as described herein.

In preferred embodiments, the one or more minimal effective fragments of the aptamers of the invention comprises a nucleic acid sequence selected from SEQ ID NOs 134, 140 or 144 or variants thereof as described herein. These exhibit an unexpected wide range of advantageous properties and have also shown to be particularly effective in ELISA-like formats. As described herein, such aptamers are capable of capable of high affinity binding to the S1 subunit whilst not binding (or binding with only low affinity) to the S2 subunit.

In preferred embodiments, the one or more minimal effective fragments of the aptamers of the invention comprise a nucleic acid sequence selected from any one of SEQ ID NOs: 146, 150 or 171 or variants thereof as described herein. These are capable of high affinity binding to the S2 subunit whilst not binding (or binding with only low affinity) to the S1 subunit. Advantageously, such aptamers may be used in combination with the S1 binding aptamers (or variants thereof) as described herein in sandwich assay platforms (e.g., ELISA like assays).

In certain embodiments, the invention provides two or more aptamers (e.g., two, three, four, five or more aptamers) capable of binding to two or more (e.g., two, three, four, five or more) different regions of the SARS-CoV-2 spike protein (including in its trimeric form) as described herein. For example, the invention provides two or more aptamers comprising any first aptamer as described herein that is capable of binding to a first region of the S1 and/or RBD of the SARS-CoV-2 spike protein (e.g. S1 subunit and/or its RBD) and any second aptamer as described herein that binds to a different or non-competing region of the SARS-CoV-2 spike protein (e.g. S1 subunit and/or its RBD). In other words, the two or more aptamers do not compete for binding to the same epitope of the SARS-CoV-2 spike protein.

In certain embodiments, the invention provides two or more aptamers, wherein a first aptamer is capable of binding to the S1 subunit of the SARS-CoV-2 spike protein and a second aptamer is capable of binding to either a different (non-overlapping) region of the S1 subunit of the SARS-CoV-2 spike protein or to the S2 subunit of the SARS-CoV-2 spike protein.

In certain embodiments, the invention provides at least a pair of aptamers comprising:
(a) a first aptamer comprising a nucleic sequence selected from any one of SEQ ID NOs 4, 8, 9, 43, 134, 144 or 140 or a sequence having at least about 85%, 90%, 95% or 99% identity or more with any one of SEQ ID NOs 4, 8, 9, 43, 134, 144 or 140; and
(b) a second aptamer comprising a nucleic sequence selected from any one of SEQ ID NOs 146, 150, 171, 177, 179, 183, 188, 190 or 191 or a sequence having at least about 85%, 90%, 95% or 99% identity or more with any one of SEQ ID NOs 146, 150, 171, 177, 179, 183, 188, 190 or 191.

In certain embodiments, the at least pair of aptamers comprises:
(a) a first and second aptamer comprising nucleic acid sequences selected from SEQ ID NO: 4 and 146, 4 and 150, 4 and 171, 4 and 177, 4 and 179, 4 and 183, 4 and 188, 4 and 190, or 4 and 191 respectively;
(b) a first and second aptamer comprising nucleic acid sequences selected from SEQ ID NO: 8 and 146, 8 and 150, 8 and 171, 8 and 177, 8 and 179, 8 and 183, 8 and 188, 8 and 190, or 8 and 191 respectively;
(c) a first and second aptamer comprising nucleic acid sequences selected from SEQ ID NO: 9 and 146, 9 and 150, 9 and 171, 9 and 177, 9 and 179, 9 and 183, 9 and 188, 9 and 190, or 9 and 191 respectively;
(d) a first and second aptamer comprising nucleic acid sequences selected from SEQ ID NO: 43 and 146, 43 and 150, 43 and 171, 43 and 177, 43 and 179, 43 and 183, 43 and 188, 43 and 190, or 43 and 191 respectively;
(e) a first and second aptamer comprising nucleic acid sequences selected from SEQ ID NO: 134 and 146, 134 and 150, 134 and 171, 134 and 177, 134 and 179, 134 and 183, 134 and 188, 134 and 190, or 134 and 191 respectively;
(f) a first and second aptamer comprising nucleic acid sequences selected from SEQ ID NO: 144 and 146, 144 and 150, 144 and 171, 144 and 177, 144 and 179, 144 and 183, 144 and 188, 144 and 190, or 144 and 191 respectively;
(g) a first and second aptamer comprising nucleic acid sequences selected from SEQ ID NO: 140 and 146, 140 and 150, 140 and 171, 140 and 177, 140 and 179, 140 and 183, 140 and 188, 140 and 190, or 140 and 191 respectively;
(h) a first and/or second aptamer comprising nucleic acid sequences having at least about 85%, 90%, 95% or 99% identity or more with any of the aptamers of (a) to (g); or
(i) a first and/or second aptamer comprising nucleic acid sequences having at least about 15, 20, 25, 30, 35, 40, 45, 50, 55, 60, 65, 70, 75 or more consecutive nucleotides of any one of the aptamers of (a) to (g).

In preferred embodiments, the one or more aptamers comprise:
(a) a nucleic acid sequence selected from SEQ ID NO: 140 and/or SEQ ID NO: 190; or
(b) one or more aptamers comprising nucleic acid sequences having at least about 85%, 90%, 95% or 99% identity or more with any of the aptamers of (a); or
(c) one or more aptamers comprising nucleic acid sequences having at least about 15, 20, 25, 30 or 31 consecutive nucleotides of any one of the aptamers of (a) to (b).

In certain embodiments, the one or more aptamers are isolated.

In certain embodiments, the one or more aptamers are DNA aptamers.

In certain embodiments, the one or more aptamers comprise a detectable label.

In certain embodiments, the invention provides one or more aptamers that compete for binding to SARS-CoV-2 spike protein (including when in its native form) with any aptamer (or variant thereof) as described herein.

In certain embodiments, the invention provides a complex comprising any one or more aptamers as described herein and one or more detectable molecules. Typically, the complex further comprises the SARS-Cov-2 virus or at least a portion thereof. For example, the complex may further comprise the S1 or S2 subunit of the spike protein, a monomer of the SARS-Cov2 spike protein, a trimer of the SARS-Cov2 spike protein or the like.

In certain embodiments, the invention provides a biosensor, assay plate or test strip comprising any one or more aptamers as described herein and one or more detectable molecules.

In certain embodiments, the invention provides a lateral flow device comprising one or more aptamers as described herein.

In certain embodiments, the invention provides a functionalised electrode or biosensor surface comprising one or more aptamers described herein.

In preferred embodiments, the functionalised electrode or biosensor comprises:
(a) an aptamer comprising a nucleic sequence selected from SEQ ID NO: 10, 20, 24 or 44; or
(b) a sequence having at least about 85%, 90%, 95% or more sequence identity with SEQ ID NO: 10, 20, 24 or 44.

As further described herein, such aptamers are particularly effective in functionalised electrodes or biosensor (e.g., wherein the aptamer is immobilised onto the surface of the electrode) due to their structural properties.

In certain embodiments, the invention provides apparatus for detecting the presence, absence or level of SARS-CoV-2 in a sample, the apparatus comprising any one or more aptamers as described herein.

In certain embodiments, the invention provides reagents for use in an ELISA or ELISA-like assay, a lateral flow device or functionalised electrode or sensor surface for detecting the presence, absence or level of SARS-CoV-2 in a sample.

In certain embodiments, the invention provides use of any one or more aptamers as described herein, any complex as described herein, any biosensor or test strip as described herein, any apparatus as described herein, any lateral flow device as described herein or any functionalised electrode as described herein for detecting, enriching, separating and/or isolating SARS-CoV-2.

In certain embodiments, the invention provides a method of detecting the presence, absence or amount of SARS-CoV-2 in a sample, the method comprising:
(i) interacting the sample with any one or more aptamers as described herein; and
(ii) detecting the presence, absence or amount of SARS-CoV-2.

In certain embodiments, the invention provides a kit for detecting, quantifying and/or enriching SARS-CoV-2, the kit comprising any one or more aptamers as described herein.

In certain embodiments, the invention provides one or more aptamers that are capable of inhibiting the interaction between the RBD of the S1 subunit within the SARS-CoV-2 spike protein with the human receptor angiotensin-converting enzyme 2 (ACE2) receptor.

In certain embodiments, the one or more aptamers capable of inhibiting the interaction between RBD of the S1 subunit with the ACE2 receptor are one or more therapeutic aptamers.

In certain embodiments, the invention provides any one or more aptamers against the S1 subunit of the SARS-CoV-2 spike protein as described herein for use as a medicament.

In certain embodiment, the invention provides any one or more aptamers as described herein against the S1 subunit of the SARS-CoV-2 spike protein for use in the treatment and/or prevention of a disease or condition in which SARS-CoV-2 is implicated.

In certain embodiments, the invention provides a pharmaceutical composition comprising any or more aptamers against the S1 subunit of the SARS-CoV-2 spike protein as described herein.

In certain embodiments, the invention provides a vaccine comprising any one or more aptamers against the S1 subunit of the SARS-CoV-2 spike protein as described herein.

In certain embodiments, the invention provides an aptamer-drug conjugate comprising any one or more aptamers against the S1 subunit of the SARS-CoV-2 spike protein as described herein.

### Detailed Description of Certain Embodiments of the Invention

### BRIEF DESCRIPTION OF THE FIGURES

Certain embodiments of the present invention will be described in more detail below, with reference to the accompanying Figures in which:

FIG. 1 shows a Selection Stringency Map used in the generation of aptamers against the S1 subunit of the SARS-CoV-2 spike protein. This table includes details of the amount of aptamer library used, amount of target protein used, the amount of counter selection target, the incubation times, the number of washes etc.

FIG. 2 shows Biolayer Interferometry data for the aptamer population C1S isolated against the S1 subunit of the spike protein from SARS-CoV-2 (COVID-19), binding to the related S1 subunits from SARS, SARS-CoV-2 (COVID-19) and MERS.

FIG. 3 shows Biolayer Interferometry data demonstrating interaction between individual aptamer clones immobilised onto sensor probes, and the S1 subunit of the spike protein from SARS-CoV-2 (COVID-19); but not significantly with the related S1 subunits from SARS and MERS. This representative data was obtained for exemplar aptamer clones S1_A8 (top) (SEQ ID NO: 9), S1_A3-A (middle) (SEQ ID NO: 4) and S1_A6 (bottom) (SEQ ID NO: 8).

FIG. 4 shows Biolayer Interferometry data demonstrating interaction between individual aptamer clones immobilised onto sensor probes, and the S1 subunit of the spike protein from SARS-CoV-2 (COVID-19), and weakly cross reacts with the related S1 subunits from MERS, but not SARS. This representative data was obtained for exemplar aptamer clones S1_A3-B (top) (SEQ ID NO: 5), S1_C1-A (middle) (SEQ ID NO: 19) and S1_08 (bottom) (SEQ ID NO: 25).

FIG. 5 shows Biolayer Interferometry data demonstrating interaction between an individual aptamer immobilised onto sensor probes, and the S1 subunit of the spike protein from SARS-CoV-2 (COVID-19), MERS and SARS. This representative data was obtained for exemplar aptamer clone S1_D11-B (SEQ ID NO: 36).

FIG. 6 shows Biolayer Interferometry assay data demonstrating concentration dependant binding of the S1 subunit of the spike protein from SARS-CoV-2 (COVID-19) to sensor probes immobilised with aptamer clones S1_A3-A (SEQ ID NO: 4) and S1_A6 (SEQ ID NO: 8); along with their overlaid kinetic fits (dashed lines). The calculated binding parameters are shown in the table accompanying each figure.

FIG. 7 shows Biolayer Interferometry assay data demonstrating concentration dependant binding of the S1 subunit of the spike protein from SARS-CoV-2 (COVID-19) to sensor probes immobilised with aptamer clones S1_A8 (SEQ ID NO: 9) and S1_F2 (SEQ ID NO: 43); along with their overlaid kinetic fits (dashed lines). The calculated binding parameters are shown in the table accompanying each figure.

FIG. 8 shows the Minimal Aptamer Fragment Identification (MAFI) Biolayer Interferometry assay data comparing interaction between a panel of immobilised fragments of aptamer S1_A8 (SEQ ID NO: 9) and the S1 subunit of the spike protein from SARS-CoV-2 (COVID-19). Fragments which retain the ability to bind the target (seen at 0-120 sec) are used to identify the minimal functional fragment. Predicted secondary structures are given for the full aptamer and the identified minimal binding fragment—“Optimer” S1_A8_F21 (SEQ ID NO: 140).

FIG. 9 shows ‘Reference corrected’ Biolayer Interferometry assay data for exemplar individual aptamer clones (upper) and their respective Optimers (lower), loaded onto BLI sensor probes; interacting with buffered saliva samples (10% and 50% (v/v) saliva; black and white traces respectively) spiked with the S1 subunit of the spike protein from SARS-CoV-2 (COVID-19), at final concentration of (0.5 μM). Data is provided for aptamers S1_A3 (SEQ ID NO: 4), S1_A6 (SEQ ID NO: 8) and S1_A8 (SEQ ID NO: 9), along with their respective Optimers S1_A3_F18 (SEQ ID NO: 134), S1_A6_F14 (SEQ ID NO: 144) and S1_A8_F21 (SEQ ID NO: 140), respectively.

FIG. 10 shows Biolayer Interferometry assay data demonstrating concentration dependent binding of the spike protein trimer from SARS-CoV-2 (COVID-19) to sensor probes immobilised with Optimers S1_A3_F18 (SEQ ID NO: 134), S1_A6_F14 (SEQ ID NO: 144), S1_A8_F21 (SEQ ID NO: 140) along with their overlaid kinetic fits. The calculated binding parameters are shown in the incorporated table.

FIG. 11 shows an indirect ELONA data demonstrating specific binding of individual aptamer clones S1_A3 (SEQ ID NO: 4), S1_A6 (SEQ ID NO: 8), S1_A8 (SEQ ID NO: 9) and Optimer S1_A3_F18 (SEQ ID NO: 134); to the S1 subunit of the spike protein from SARS-CoV-2 (COVID-19) (left bar). No binding is seen to the related S1 subunits from SAR-CoV (middle bar) or MERS (right bar).

FIG. 12 shows a Selection Stringency Map used in the generation of aptamers against the S2 subunit of the spike protein from SARS-CoV-2 (COVID-19). Similar to FIG. 1, this table includes details of the amount of aptamer library used, amount of target protein used, the amount of counter selection target, the incubation times, the number of washes etc.

FIG. 13 shows Biolayer Interferometry assay data demonstrating concentration dependent binding of the spike protein trimer from SARS-CoV-2 (COVID-19) to sensor probes immobilised with Optimers S2_A2_F12 (SEQ ID NO: 179), S2_B1_F12 (SEQ ID NO: 188), S2_G1_F21 (SEQ ID NO: 190) along with their overlaid kinetic fits. The calculated binding parameters are shown in the incorporated table.

FIG. 14 shows Biolayer Interferometry assay data demonstrating concentration dependent binding of the S1 subunit of the spike protein from SARS-CoV-2 (COVID-19) WT (SARS-CoV-2 WT) and Denmark, Kent, South Africa and Brazil variants of concern (SARS-CoV-2 D14G, SARS-CoV-2 B.1.1.7, SARS-CoV-2 B.1.351, and SARS-CoV-2 P.1, respectively), to sensor probes immobilised with Optimer S1_A8_F21 (SEQ ID NO: 140), along with their respective overlaid kinetic fits. The calculated binding parameters are shown in the incorporated table.

FIG. 15 shows Biolayer Interferometry assay data demonstrating concentration dependent binding of the irradiated viral material from SARS-CoV-2 WT or the Kent or South Africa variants of concern (SARS-CoV-2 B.1.1.7 and SARS-CoV-2 B.1.351, respectively), to sensor probes immobilised with Optimers S1_A8_F21 (SEQ ID NO: 140) or S2_G1_F21 (SEQ ID NO: 190); (upper and lower respectively).

FIG. 16 shows Sandwich ELONA data demonstrating concentration dependent detection of SARS-CoV-2 trimer, when the S1 binding Optimer S1_A3_F18 (SEQ ID NO: 134) is used as a capture reagent and SARS-CoV-2 S2 binding aptamer clones S2_A2 (SEQ ID NO: 146) (middle left bar), S2_B1 (SEQ ID NO: 150) (middle right bar), S2_G1 (SEQ ID NO: 171) (right bar) or the parent aptamer pool S2_8R (left bar) are used as detection reagents.

FIG. 17 shows Sandwich ELONA data demonstrating concentration dependent detection of SARS-CoV-2 trimer, when the S1 aptamer clone S1_A3 (SEQ ID NO: 4); or Optimer S1_A3_F18 (SEQ ID NO: 134) are used as a capture reagent and S2 binding Optimers S2_A2_F17 (SEQ ID NO: 177) (right bar), S2_B1_F18 (SEQ ID NO: 183) (middle bar) or S2_G1_F22 (SEQ ID NO: 191) (left bar); are used as detection reagents.

FIG. 18 shows concentration dependent detection of irradiated SARS-CoV-2 viral material, in a Lateral Flow Device using the S1 binding Optimer ‘S1_A8_F21’ (SEQ ID NO: 140) immobilised in the ‘Test line’ and S2 binding Optimer ‘S2_G1_F21’ (SEQ ID NO: 190) immobilised on the Gold Nanoparticles used for detection. Mock nasal swab samples were spiked with irradiated SARS-CoV-2 at 1×10⁵-3×10³ PFU/ml. No ‘Test line’ is seen in the buffer alone ‘negative control’ (far right), demonstrating that Test line formation is specific to the viral material.

### DETAILED DESCRIPTION

Further features of certain embodiments of the present invention are described below. The practice of embodiments of the present invention will employ, unless otherwise indicated, conventional techniques of molecular biology, microbiology, recombinant DNA technology and immunology, which are within the skill of those working in the art.

Most general molecular biology, microbiology recombinant DNA technology and immunological techniques can be found in Sambrook et al, Molecular Cloning, A Laboratory Manual (2001) Cold Harbor-Laboratory Press, Cold Spring Harbor, N.Y. or Ausubel et al., Current protocols in molecular biology (1990) John Wiley and Sons, N.Y. Unless defined otherwise, all technical and scientific terms used herein have the same meaning as commonly understood by one of ordinary skill in the art to which this disclosure belongs. For example, the Concise Dictionary of Biomedicine and Molecular Biology, Juo, Pei-Show, 2nd ed., 2002, CRC Press; The Dictionary of Cell and Molecular Biology, 3rd ed., Academic Press; and the Oxford University Press, provide a person skilled in the art with a general dictionary of many of the terms used in this disclosure.

Units, prefixes and symbols are denoted in their Système International de Unitese (SI) accepted form. Numeric ranges are inclusive of the numbers defining the range. Unless otherwise indicated, amino acid sequences are written left to right in amino to carboxy orientation and nucleic acid sequences are written left to right in 5′ to 3′ orientation.

As used herein, the term “antigen” refers to a molecule, generally a toxin or other foreign substance, capable of inducing an immune response in a subject organism (namely, a host), such as a human or an animal. Sometimes, antigens are produced by a pathogen, such as a bacteria or virus; in other instances, the antigen is produced by the host organism itself, the antigen may be responsible or contribute to a disease or condition in the host organism.

Furthermore, as used herein, the term “antigen” refers to a molecular target of an aptamer, which is expressed in a tissue, a cell or a virus and/or secreted in body fluids such as urine, saliva, nasal swab, sputum, tear, blood, seminal and cerebrospinal fluids.

As used herein, the term “virus” refers to a small infectious agent. Generally, viruses are non-living complex molecules that can only replicate inside the living cells of host organisms. Viruses infect a variety of organisms such as animals, plants, humans, bacteria, and so forth, causing various diseases and conditions.

As used herein, the term “coronavirus” refers to a virus from the Coronaviridae family. Typically, these large single-stranded RNA viruses comprise a lipid envelope studded with club-shaped spike proteins. These viruses may cause diseases in mammals and birds. Specifically, these viruses may cause respiratory tract infections in humans, ranging from mild to lethal.

In the following, the invention will be explained in more detail by means of non-limiting examples of specific embodiments. In the example experiments, standard reagents and buffers free from contamination are used.

SARS-CoV-2 Spike Protein

In certain embodiments, the invention provides one or more aptamers capable of binding to the SARS-CoV-2 spike protein (including in its native trimeric form).

The entry of the SARS-CoV-2 virus into human cells is thought to be mediated by the transmembrane spike protein, which is located on the virion and protrudes out from the viral surface. The spike protein is a homotrimeric glycoprotein (e.g. “trimer” as referred to herein), each monomer is around 180 kDa and comprises two functional subunits (S1 and S2), which are responsible for host cell receptor recognition (S1) and viral-cell membrane fusion (S2) respectively. The spike S1 subunit includes the RBD, which may recognize and bind to human receptor angiotensin-converting enzyme 2 (ACE2). The direct binding of the RBD to ACE2's peptidase domain (PD) may enable high affinity binding by the spike protein.

Although the spike protein's RBD may be the most variable part of the coronavirus genome, the amino acid residues required for ACE2 binding are conserved between SARS-CoV and SARS-CoV-2.

In certain embodiments, the aptamers of the invention are capable of binding to SARS-CoV-2 and are also capable of binding to other coronaviruses (e.g., SARS-CoV and/or MERS-CoV). In other words, the aptamers of the invention may cross-react with homologous coronavirus spike proteins (such as SARS-CoV and MERS-CoV spike proteins).

In certain embodiments, the aptamers of the invention are capable of specifically binding to SARS-CoV-2, but do not specifically bind to other coronaviruses (e.g. SARS-CoV and/or MERS-CoV). In other words, the aptamers of the invention are highly selective for the SARS-CoV-2 spike protein and may not cross-react with homologous coronavirus spike proteins (such as SARS-CoV and MERS-CoV's spike proteins).

In certain embodiments, the invention provides one or more aptamers capable of specifically binding to the full-length SARS-CoV-2 spike protein as set forth in SEQ ID NO: 117 or variants thereof. In certain embodiments, the aptamers of the invention specifically bind to a protein having at least about 80%, at least about 85%, at least about 90%, at least about 95%, at least about 96%, at least about 97%, at least about 98%, at least about 99% or more sequence identity to SEQ ID NO: 117.

In certain embodiments, the invention provides one or more aptamers capable of specifically binding to the full-length SARS-CoV-2 spike protein (i.e., as set forth in SEQ ID NO: 117 or variants thereof) within its natural conformation as part of a trimer (i.e., the homotrimeric glycoprotein).

In certain embodiments, the invention provides one or more aptamers capable of specifically binding to amino acids 1 to 1213 of SEQ ID NO: 117 (or variants thereof).

In certain embodiments, the invention provides one or more aptamers capable of specifically binding to amino acids 1214 to 1273 of SEQ ID NO: 117 (or variants thereof).

In certain embodiments, the invention provides one or more aptamers capable of specifically binding to amino acids 686 to 1213 of SEQ ID NO: 117 (or variants thereof).

In certain embodiments, the invention provides one or more aptamers capable of specifically binding to the S2 subunit of the SARS-CoV-2 spike protein as set forth in SEQ ID NO: 119 and/or variants thereof. In certain embodiments, the aptamers of the invention specifically bind to a protein having at least about 90%, at least about 95%, at least about 96%, at least about 97%, at least about 98%, at least about 99% or more sequence identity to SEQ ID NO: 119.

In certain embodiments, the invention provides one or more aptamers capable of specifically binding to amino acids 1 to 685 of SEQ ID NO: 117 (or variants thereof).

In certain embodiments, the invention provides one or more aptamers capable of specifically binding to amino acids 16 to 685 of SEQ ID NO: 117 (or variants thereof).

In certain embodiments, the invention provides one or more aptamers capable of specifically binding to amino acids 300 to 600 of SEQ ID NO: 117 (or variants thereof).

In certain embodiments, the invention provides one or more aptamers capable of specifically binding to amino acids 319 to 541 of SEQ ID NO: 117 (or variants thereof).

In certain embodiments, the invention provides one or more aptamers capable of specifically binding to amino acids 1 to 528 of SEQ ID NO: 119 (or variants thereof).

In certain embodiments, the invention provides one or more aptamers capable of specifically binding to amino acids 1 to 528 of SEQ ID NO: 119 (e.g., Ser686 to Pro1213 of the SARS-CoV-2 spike protein S2 subunit amino acid sequence) (or variants thereof).

In certain embodiments, the invention provides one or more aptamers capable of specifically binding to a variant of SEQ ID NO: 117 comprising one or more of the following point mutations:
(a) Alanine to Serine at position 435 (A435S);
(b) Phenylalanine to Leucine at position 342 (F342L);
(c) Lysine to Arginine at position 458 (K458R);
(d) Asparagine to Aspartic acid at position 354 (N354D);
(e) Valine to Phenylalanine at position 367 (V367F);
(f) Valine to Alanine at position 483 (V483A);
(g) Aspartic acid to Glycine at position 614 (D614G); and/or
(h) Glutamic acid to Lysine at position 484 (E484K).

In certain embodiments, the invention provides one or more aptamers capable of binding to one or more emerging SARS-CoV-2 variants. In certain embodiments, the aptamers are capable of binding to one or more SARS-CoV-2 variants that are more capable of evading the immune system and/or have different binding affinity or specificity for host cell receptors as compared to the original lineage of SARS-CoV-2 (also referred to as “Wuhan-Hu-1”).

In preferred embodiments, the aptamers of the invention are capable of binding to the B.1.1.7 variant (also referred to as the “UK variant”), B.1.351 variant (also referred to as the “South Africa variant” or “SA variant”), P.1 variant (also referred to as the “Brazil variant”) and/or the D614G variant (also referred to as the “Denmark variant”.

In certain embodiments, the aptamers of the invention are capable of binding to additional dominant emerging variants. For example, the aptamers of the invention may also be capable of binding to the B.1.617.2 (Delta or Indian variant) or any other variants that may arise.

In certain embodiments, the invention provides one or more aptamers capable of binding to one or more of the equivalent point mutations in SEQ ID NO:118 (e.g., corresponding to Val16-Arg685 of SEQ ID NO:117).

In certain embodiments, the invention provides one or more aptamers capable of specifically binding to the S1 subunit of the SARS-CoV-2 spike protein as set forth in SEQ ID NO: 118 or variants thereof. In certain embodiments, the aptamers of the invention specifically bind to a protein having at least about 80%, at least about 85%, at least about 90%, at least about 95%, at least about 96%, at least about 97%, at least about 98%, at least about 99% or more sequence identity to SEQ ID NO: 118.

In certain embodiments, the invention provides one or more aptamers capable of specifically binding to one or more amino acids of the S1 subunit of the SARS-CoV-2 spike protein that are outside of the RBD of the S1 subunit of the SARS-CoV-2 spike protein. For example, in certain embodiments the aptamers are capable of specifically binding to one or more amino acids 1 to 318 or 542 to 685 of the full-length spike protein as set forth in SEQ ID NO: 117. In certain embodiments, the aptamers are capable of specifically binding to one or more amino acids 1 to 303 or 542 to 685 of the S1 subunit as set forth in SEQ ID NO: 118.

In certain embodiments, the invention provides one or more aptamers that are capable of specifically binding to:
(a) full-length SARS-CoV-2 spike protein as set forth in SEQ ID NO: 117 or variants thereof; and/or
(b) the S1 subunit of the SARS-CoV-2 spike protein as set forth in SEQ ID NO: 118 or variants thereof;
wherein the aptamers are not capable of specifically binding to the RBD of the S1 subunit of the SARS-CoV-2 spike protein as set forth in SEQ ID NO: 120 or variants thereof.

In certain embodiments, the aptamers of the invention do not specifically bind to a protein having at least about 80%, at least about 85%, at least about 90%, at least about 95%, at least about 96%, at least about 97%, at least about 98%, at least about 99% or more sequence identity to SEQ ID NO: 120.

In certain embodiments, the invention provides one or more aptamers capable of specifically binding to one or more amino acids of the S1 subunit of the SARS-CoV-2 spike protein that are within the RBD of the S1 subunit of the SARS-CoV-2 spike protein. For example, in certain embodiments the aptamers are capable of specifically binding to one or more amino acids 319 to 541 of the full-length spike protein as set forth in SEQ ID NO: 117. In certain embodiments, the aptamers are capable of specifically binding to one or more amino acids 304 to 526 of the S1 subunit as set forth in SEQ ID NO: 118.

In certain embodiments, the invention provides one or more aptamers that are capable of specifically binding to:
(a) full-length SARS-CoV-2 spike protein as set forth in SEQ ID NO: 117 or variants thereof; and/or
(b) the S1 subunit of the SARS-CoV-2 spike protein as set forth in SEQ ID NO: 118 or variants thereof;
wherein the aptamers are capable of specifically binding to the RBD of the S1 subunit of the SARS-CoV-2 spike protein as set forth in SEQ ID NO: 120 or variants thereof.

In certain embodiments, the invention provides one or more aptamers capable of specifically binding to the receptor-binding domain (RBD) of the S1 subunit of the SARS-CoV-2 spike protein as set forth in SEQ ID NO: 120 or variants thereof. In certain embodiments, the aptamers of the invention specifically bind to a protein having at least about 80%, at least about 85%, at least about 90%, at least about 95%, at least about 96%, at least about 97%, at least about 98%, at least about 99% or more sequence identity to SEQ ID NO: 120.

In certain embodiments, the aptamers of the invention are capable of specifically binding to the S1 subunit of the SARS-CoV-2 spike protein, including the RBD of the S1 subunit.

An aptamer that binds “specifically” to the SARS-CoV-2 spike protein (e.g. the S1 subunit of the SARS-Cov-2 spike protein and/or its RBD) is an aptamer which binds with preferential or high affinity to the SARS-CoV-2 spike protein (or subunit and/or RBD thereof) but does not bind (or binds with lower affinity) to the homologous spike protein (or subunit and/or RBD) of other coronaviruses.

In certain embodiments, the aptamers of the invention bind with preferential or high affinity to SARS-CoV-2 spike protein S1 (and/or its RBD), and do not bind (or bind with only low affinity) to homologous spike proteins of other coronaviruses.

In certain embodiments, the aptamers of the invention may not bind (or bind with only low affinity) to the SARS coronavirus (SARS-CoV, as identified in 2003) and/or Middle East respiratory syndrome coronavirus (MERS-CoV).

In certain embodiments, an aptamer that binds with preferential or high affinity to the SARS-Cov-2 spike protein may bind with at least about 2×, 3×, 4×, 5×, 10×, 20×, 50×, 100× or 1000× more affinity as compared to binding to the equivalent protein of other coronoaviruses (e.g. SARS-CoV and/or MERS-CoV).

In certain embodiments, a “strong” binder as described herein is an aptamer that has a target interaction greater than 1 nm based on the signal response in a BLI assay. In certain embodiments, a “moderate” binder as described herein is an aptamer that has a target interaction of between 0.5 nm to 1 nm based on the signal response in a BLI assay. In certain embodiments, a “low” (or no significant) binder as described herein is an aptamer that has a target interaction of less than 0.5 nm (e.g., less than 0.3 nm) based on the signal response in a BLI assay.

In certain embodiments, the aptamer of the invention may not bind (or binds with only low affinity) to the full-length SARS-CoV spike protein as set forth in SEQ ID NO: 121 or variants thereof. In certain embodiments, the aptamers of the invention may not bind (or bind with only low affinity) to a protein having at least about 85%, at least about 90%, at least about 95%, at least about 96%, at least about 97%, at least about 98%, at least about 99% or more sequence identity to SEQ ID NO: 121.

In certain embodiments, the aptamers of the invention may not bind (or bind with only low affinity) to the S1 subunit of the SARS-CoV spike protein as set forth in SEQ ID NO: 122 or variants thereof. In certain embodiments, the aptamers of the invention may not bind (or bind with only low affinity) to a protein having at least about 85%, at least about 90%, at least about 95%, at least about 96%, at least about 97%, at least about 98%, at least about 99% or more sequence identity to SEQ ID NO: 122.

In certain embodiments, the aptamers of the invention may not bind (or bind with only low affinity) to the S2 subunit of the SARS-CoV spike protein as set forth in SEQ ID NO: 123 or variants thereof. In certain embodiments, the aptamers of the invention may not bind (or bind with only low affinity) to a protein having at least about 90%, at least about 91%, at least about 95%, at least about 96%, at least about 97%, at least about 98%, at least about 99% or more sequence identity to SEQ ID NO: 123.

In certain embodiments, the aptamers of the invention may not bind (or bind with only low affinity) to the RBD of the S1 subunit of the SARS-CoV spike protein as set forth in SEQ ID NO: 124 or variants thereof. In certain embodiments, the aptamers of the invention may not bind (or bind with only low affinity) to a protein having at least about 85%, at least about 90%, at least about 95%, at least about 96%, at least about 97%, at least about 98%, at least about 99% or more sequence identity to SEQ ID NO: 124.

In certain embodiments, the aptamers of the invention may not bind (or bind with only low affinity) to the full-length MERS-CoV spike protein as set forth in SEQ ID NO: 125 or variants thereof. In certain embodiments, the aptamers of the invention may not bind (or bind with only low affinity) to a protein having at least about 80%, at least about 85%, at least about 90%, at least about 95%, at least about 96%, at least about 97%, at least about 98%, at least about 99% or more sequence identity to SEQ ID NO: 125.

In certain embodiments, the aptamers of the invention may not bind (or bind with only low affinity) to the S1 subunit of the MERS-CoV spike protein as set forth in SEQ ID NO: 126 or variants thereof. In certain embodiments, the aptamers of the invention may not bind (or bind with only low affinity) to a protein having at least about 80%, at least about 85%, at least about 90%, at least about 95%, at least about 96%, at least about 97%, at least about 98%, at least about 99% or more sequence identity to SEQ ID NO: 126.

In certain embodiments, the aptamers of the invention may not bind (or bind with only low affinity) to the S2 subunit of the MERS-CoV spike protein as set forth in SEQ ID NO: 127 or variants thereof. In certain embodiments, the aptamers of the invention may not bind (or bind with only low affinity) to a protein having at least about 80%, at least about 85%, at least about 90%, at least about 95%, at least about 96%, at least about 97%, at least about 98%, at least about 99% or more sequence identity to SEQ ID NO: 127.

In certain embodiments, the aptamers of the invention may not bind (or bind with only low affinity) to the RBD of the S1 subunit of the MERS-CoV spike protein as set forth in SEQ ID NO: 128 or variants thereof. In certain embodiments, the aptamers of the invention may not bind (or bind with only low affinity) to a protein having at least about 80%, at least about 85%, at least about 90%, at least about 95%, at least about 96%, at least about 97%, at least about 98%, at least about 99% or more sequence identity to SEQ ID NO: 128.

In certain embodiments, the aptamers of the invention bind with high affinity to the SARS-CoV-2 spike protein (including the S1 subunit and/or its RBD), and also bind with high affinity to homologous spike proteins of other coronaviruses.

In certain embodiments, the aptamers of the invention bind with high affinity to the SARS-CoV-2 spike protein (including the S1 subunit and/or its RBD), and also bind with high affinity to homologous spike protein (including the S1 subunit and/or its RBD) of the SARS coronavirus (SARS-CoV, as identified in 2003) and/or Middle East respiratory syndrome coronavirus (MERS-CoV).

In certain embodiments, the aptamers of the invention may bind (or bind with high affinity) to the full-length SARS-CoV spike protein as set forth in SEQ ID NO: 121 or variants thereof. In certain embodiments, the aptamers of the invention may bind (or bind with high affinity) to a protein having at least about 80%, at least about 85%, at least about 90%, at least about 95%, at least about 96%, at least about 97%, at least about 98%, at least about 99% or more sequence identity to SEQ ID NO: 121.

In certain embodiments, the aptamers of the invention may bind (or bind with high affinity) to the S1 subunit of the SARS-CoV spike protein as set forth in SEQ ID NO: 122 or variants thereof. In certain embodiments, the aptamers of the invention may bind (or bind with high affinity) to a protein having at least about 80%, at least about 85%, at least about 90%, at least about 95%, at least about 96%, at least about 97%, at least about 98%, at least about 99% or more sequence identity to SEQ ID NO: 122.

In certain embodiments, the aptamers of the invention may bind (or bind with high affinity) to to the S2 subunit of the SARS-CoV spike protein as set forth in SEQ ID NO: 123 or variants thereof. In certain embodiments, the aptamers of the invention may bind (or bind with high affinity) to a protein having at least about 90%, at least about 95%, at least about 96%, at least about 97%, at least about 98%, at least about 99% or more sequence identity to SEQ ID NO: 123.

In certain embodiments, the aptamers of the invention may bind (or bind with high affinity) to the RBD of the S1 subunit of the SARS-CoV spike protein as set forth in SEQ ID NO: 124 or variants thereof. In certain embodiments, the aptamers of the invention may bind (or bind with high affinity) to a protein having at least about 80%, at least about 85%, at least about 90%, at least about 95%, at least about 96%, at least about 97%, at least about 98%, at least about 99% or more sequence identity to SEQ ID NO: 124.

In certain embodiments, the aptamers of the invention may bind (or bind with high affinity) to the full-length MERS-CoV spike protein as set forth in SEQ ID NO: 125 or variants thereof. In certain embodiments, the aptamers of the invention may bind (or bind with high affinity) to a protein having at least about 80%, at least about 85%, at least about 90%, at least about 95%, at least about 96%, at least about 97%, at least about 98%, at least about 99% or more sequence identity to SEQ ID NO: 125.

In certain embodiments, the aptamers of the invention may bind (or bind with high affinity) to the S1 subunit of the MERS-CoV spike protein as set forth in SEQ ID NO: 126 or variants thereof. In certain embodiments, the aptamers of the invention may bind (or bind with high affinity) to a protein having at least about 80%, at least about 85%, at least about 90%, at least about 95%, at least about 96%, at least about 97%, at least about 98%, at least about 99% or more sequence identity to SEQ ID NO: 126.

In certain embodiments, the aptamers of the invention may bind (or bind with high affinity) to the S2 subunit of the MERS-CoV spike protein as set forth in SEQ ID NO: 127 or variants thereof. In certain embodiments, the aptamers of the invention may bind (or bind with high affinity) to a protein having at least about 80%, at least about 85%, at least about 90%, at least about 95%, at least about 96%, at least about 97%, at least about 98%, at least about 99% or more sequence identity to SEQ ID NO: 127.

In certain embodiments, the aptamers of the invention may bind (or bind with high affinity) to the RBD of the S1 subunit of the MERS-CoV spike protein as set forth in SEQ ID NO: 128 or variants thereof. In certain embodiments, the aptamers of the invention may bind (or bind with high affinity) to a protein having at least about 80%, at least about 85%, at least about 90%, at least about 95%, at least about 96%, at least about 97%, at least about 98%, at least about 99% or more sequence identity to SEQ ID NO: 128.

As used herein, the term “high affinity” is understood to mean, for example, an aptamer that binds to the protein with a binding dissociation equilibrium constant (K_{D}) of less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 0.9 nM, less than about 0.8 nM, less than about 0.7 nM, less than about 0.6 nM, less than about 0.5 nM, less than about 0.4 nM, less than about 0.3 nM, less than about 0.2 nM, less than about 0.1 nM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 3 pM, less than about 2 pM, less than about 1 pM or less.

In certain embodiments, the aptamers of the invention are capable of specifically binding to the SARS-CoV-2 spike protein (including the S1 subunit and/or its RBD and/or the S2 subunit) with a K_{D} of less than about 100 nM or less. For example, the aptamers of the invention preferably have a K_{D} of less than about 90 nM, 80 nM, 70 nM, 60 nM, 50 nM, 40 nM, 30 nM, 20 nM, 10 nM or less against the SARS-CoV-2 spike protein in its native trimeric form. In certain embodiments, the aptamers of the invention have a K_{D} between about 20-40 nM against the SARS-CoV-2 spike protein in its native trimeric form.

The binding affinity of aptamers may be measured by any method known to a person skilled in the art, including, for example, surface plasmon resonance (SPR), Biolayer Interferometry (BLI), ELISA, fluorescence assays such as fluorescence anisotropy or fluorescence polarisation, Microscale Thermophoresis or the like.

As used herein, the term “low affinity” is understood to mean, for example, an aptamer that binds to the protein with a binding dissociation equilibrium constant (K_{D}) of more than about 1 μM, more than about 2 μM, more than about 3 μM, more than about 4 μM, more than about 5 μM, more than about 6 μM, more than about 7 μM, more than about 8 μM, more than about 9 μM, more than about 10 μM or has no detectable response.

In certain embodiments, the aptamers of the invention have a rapid association rate (e.g., between about 0-120 seconds) and a slow dissociation rate (e.g., between about 120-240 seconds). Methods of measuring the association rate constant (Kₐ) and dissociation rate constant (K_{d}) are also well described in the art.

In certain embodiments, the aptamers of the invention are capable of binding (e.g., bind with high affinity) to SARS-CoV-2 proteins (e.g., the S1 subunit including its RBD and/or the S2 subunit) as expressed in any type of cell. Suitable cells may include prokaryotic cells such as bacterial cells (e.g., Escherichia coli or the like) or eukaryotic cells such as insects (e.g., Baculovirus insect cells or the like).

In certain embodiments, the aptamers of the invention are capable of binding (e.g., bind with high affinity) to SARS-CoV-2 proteins (e.g., the S1 subunit including its RBD and/or the S2 subunit) as expressed in mammalian cells (e.g., CHO cells, HEK293 cells, or the like). Typically, the aptamers of the invention are capable of binding with high affinity to SARS-CoV-2 spike protein that is produced in a mammalian (e.g. human) host cell during a COVID-19 viral infection.

Advantageously, the aptamers of the invention are capable of binding (e.g., bind with high affinity) to SARS-CoV-2 spike protein that has authentic protein folding, glycosylation and/or post-translational modifications as compared to naturally occurring SARS-CoV-2 spike protein produced in its host (e.g. human) cell environment. The aptamers of the invention (e.g. raised against target SARS-CoV-2 spike protein expressed in mammalian cell lines) may therefore display improved native target protein binding as compared to any other types of aptamer raised against target SARS-CoV-2 spike protein expressed in non-mammalian cell lines (e.g. prokaryotic or insect cell expression systems as further described herein).

The SARS-CoV-2 spike protein may undergo several post-translational modifications (PTMs), including N-linked glycosylation and palmitoylation. Aptly, the aptamers of the invention are capable of binding (e.g., bind with high affinity) to post-translationally modified (e.g. specific N-glycosylated) SARS-CoV-2 spike protein. In such embodiments, the aptamers of the invention may not bind (or bind with only low affinity) to non post-translationally modified SARS-CoV-2 spike protein (e.g. spike protein instead produced using prokaryotic or insect expression systems).

Aptamers

The aptamers described herein are small artificial ligands, comprising DNA, RNA or modifications thereof, capable of specifically binding to SARS-CoV-2 spike protein with high affinity and specificity. Typically, the aptamers of the invention are capable of specifically binding to SARS-CoV-2 spike protein S1 and/or the RBD of this S1 subunit and/or the S2 subunit.

As used herein, “aptamer”, “nucleic acid molecule” or “oligonucleotide” are used interchangeably to refer to a non-naturally occurring nucleic acid molecule that has a desirable action on a target molecule (i.e., SARS-CoV-2 spike protein, especially S1 and/or its RBD and/or S2).

The aptamers of the invention may be DNA aptamers. For example, the aptamers may be formed from single-stranded DNA (ssDNA). Alternatively, the aptamers of the invention may be RNA aptamers. For example, the aptamers can be formed from single-stranded RNA (ssRNA). The aptamers of the invention may comprise modified nucleic acids as described herein.

In preferred embodiments, the aptamers of the invention are DNA aptamers (e.g., ssDNA aptamers).

In certain embodiments, the aptamers of the invention are prepared using principles of in vitro selection known in the art, that include iterative cycles of target binding, partitioning and preferential amplification of target binding sequences.

In certain embodiments, the invention provides a single aptamer. In certain embodiments, the invention provides more than one aptamer, e.g., two, three, four, five or more aptamers. In certain embodiments, the invention provides at least two or more aptamers (e.g., aptamer pair, triplet or more) capable of binding to two or more different regions of the SARS-CoV-2 spike protein as described herein. In other words, the two or more aptamers do not compete for binding to the same epitope of the SARS-CoV-2 spike protein.

In certain embodiments, the invention provides at least two or more aptamers (e.g., aptamer pair, triplet or more) capable of binding to two or more different regions of the S1 subunit of the SARS-CoV-2 spike protein as described herein.

In certain embodiments, the invention provides at least two or more aptamers (e.g., aptamer pair, triplet or more) capable of binding to two or more different regions of the S2 subunit of the SARS-CoV-2 spike protein as described herein.

In preferred embodiments, the invention provides at least two or more aptamers (e.g., aptamer pair, triplet or more) wherein at least one aptamer is capable of binding to the S1 subunit and at least one aptamer is capable of binding to the S2 subunit of the SARS-CoV-2 spike protein as described herein.

In certain embodiments, the aptamer against the S1 subunit is used for “capture”, whilst the aptamer against the S2 subunit is used for “detection” as further described herein. Alternatively, the aptamer against the S2 subunit may be used for “capture”, whilst the aptamer against the S1 subunit is used for “detection” as further described herein.

As described herein, a “first aptamer” of the invention may be used for capture or detection. If the first aptamer is used for capture, the “second aptamer” may be used for detection. If the first aptamer is used for detection, the second aptamer may be used for capture.

In certain embodiments, the aptamers are selected from a nucleic acid molecule library such as a single-stranded DNA or RNA nucleic acid molecule library. Typically, the aptamers are selected from a library that is designed such that any selected aptamers need little to no adaptation to convert into any of the listed assay formats. In certain embodiments, the library comprises at least the following functional parts: a first primer binding region (P1), at least one randomised region (R) and a second primer binding region (P2).

Aptly, at least a portion of the randomised region (R) is involved in target molecule binding. The randomised region may be any suitable length. Typically, the randomised region is about 30 to 60 nucleic acid bases in length. For example, the randomised region may be about 40 nucleotides in length.

Aptly, the primer regions may serve as primer binding sites for PCR amplification of the library and selected aptamers. Once selected, the aptamer may be further modified before being used e.g., to remove one or both primer sequences and/or parts of the randomised region not required for target binding.

The skilled person would understand different primer sequences can be selected depending, for example, on the starting library and/or aptamer selection protocol. For example, the aptamers of the invention may comprise SEQ ID NO: 114 and/or 115. Thus, in certain embodiments the aptamers of the invention may be selected using the forward and reverse primer pair as set forth in SEQ ID NO: 114 and 116. In any of the aptamer sequences described herein, these primer sequences may be replaced with any alternative suitable primer sequence.

The first primer region and/or second region may comprise a detectable label as described herein. For example, the first and/or second primer region may be fluorescently (e.g., FAM)-labelled. In certain embodiments, the first and/or second primer region primer are phosphate (PO₄) labelled.

In certain embodiments, the aptamer of the invention further comprises a linker sequence. For example, aptamers of the invention may be immobilised to a support via one or more linker sequences as described herein.

In certain embodiments, the aptamers of the invention comprise or consist of a nucleic acid sequence selected from any one of SEQ ID NOs: 1 to 113 or fragments thereof (e.g., any one of SEQ ID NOs 129 to 144).

In certain embodiments, the aptamers of the invention comprise or consist of a nucleic acid sequence selected from any one of SEQ ID NOs: 145 to 176 or fragments thereof (e.g., any one of SEQ ID NOs 177 to 192).

S1 Aptamers

In certain embodiments, the aptamers of the invention comprise or consist of a nucleic acid sequence selected from any one of SEQ ID NOs 1 to 53 (i.e., aptamers generated against the S1 subunit of the SARS-CoV-2 spike protein).

In certain embodiments, the aptamers of the invention comprise or consist of a nucleic acid sequence selected from any one of SEQ ID NOs 54 to 113 (i.e., aptamers generated against the RBD of the S1 subunit of the SARS-CoV-2 spike protein).

In certain embodiments, the aptamers of the invention comprise or consist of any one of SEQ ID NOs 1, 2, 4 to 6, 8 to 14, 16 to 26, 28 to 46 or 48 to 49 (or variants thereof). The aptamers of the invention may comprise or consist of any one of SEQ ID NOs 4, 5, 6, 14, 17, 18, 21, 29, 32, 38, 45 or 50 (or variants thereof). As described further herein, these aptamers are capable of binding to an accessible region of the RBD within the subunit S1 of the SARS-CoV-2 spike protein (i.e., capable of binding to the RBD within its native conformation as part of the S1 subunit).

In certain embodiments, the aptamers of the invention comprise or consist of any one of SEQ ID NOs 1, 2, 4 to 6, 8 to 12, 14, 16 to 17, 19 to 25, 29, 31 to 32, 35 to 38, 43 to 45 or 48 to 49 (or variants thereof). For example, the aptamers of the invention may comprise or consist of any one of SEQ ID NOs 4, 5, 6, 14, 17, 21, 29, 32, 38 or 45. As described further herein, these aptamers are capable of binding with high affinity to an accessible region of the RBD within the subunit S1 of the SARS-CoV-2 spike protein.

In certain embodiments, the aptamers of the invention comprise or consist of a nucleic acid sequence selected from any one of SEQ ID NOs 2, 4, 6, 8, 9, 13, 16, 17, 18, 20, 21, 22, 23, 24, 26, 31, 34, 35, 40 to 46, 48 or 49 (or variants thereof). For example, the aptamers of the invention may comprise or consist of any one of SEQ ID NOs 4, 6, 17, 18, 21 or 45. As described further herein, these aptamers are capable of specifically binding to an accessible region of the RBD within the subunit S1 of the SARS-CoV-2 spike protein. In other words, these aptamers bind to an accessible region of the RBD within the S1 subunit of the SARS-CoV-2 spike protein but do not bind (or bind with low affinity) to the homologous protein from MERS-CoV and/or SARS-CoV.

In certain embodiments, the aptamers of the invention comprise or consist of any one of SEQ ID NOs 2, 4, 6, 8, 9, 16, 17, 20, 21, 22, 23, 24, 31, 35, 43 to 45, 48 or 49 (or variants thereof). For example, the aptamers of the invention comprise or consist of any one of SEQ ID NOs 4, 8, 9 or 43 (or variants thereof). As described further herein, these aptamers specifically bind with high affinity to the S1 subunit of the SARS-CoV-2 spike protein.

In certain embodiments, the aptamers of the invention comprise or consist of any one of SEQ ID NOs 4, 8, 9, 16, 21, 23, 31, 43 or 48 (or variants thereof). As described further herein, these aptamers specifically bind to SARS-CoV-2 spike protein in a saliva sample.

In certain embodiments, the aptamers of the invention comprise or consist of any one of SEQ ID NOs 4, 8, 9 (or variants thereof). As described further herein, these aptamers specifically bind with high affinity to the S1 subunit of the SARS-CoV-2 spike protein. Further, these aptamers specifically bind to SARS-CoV-2 spike protein in its native trimeric form and are particularly effective in an ELISA like assay format.

In certain embodiments, the aptamers of the invention comprise or consist of any one of SEQ ID NOs 21, 23, 24 or 31 (or variants thereof). As described further herein, these aptamers also specifically bind to SARS-CoV-2 spike protein in its native trimeric form.

In certain embodiments, the aptamers of the invention comprise or consist of any one of SEQ ID NOs 9, 21, 23 or 31 (or variants thereof). As described further herein, these aptamers specifically bind to SARS-CoV-2 spike protein in its native trimeric form and specifically bind to SARS-CoV-2 spike protein in a saliva sample.

In certain embodiments, the aptamers of the invention comprise or consist of any one of SEQ ID NOs 1, 5, 10, 11, 12, 14, 19, 25, 28 to 30, 32, 33, 36 to 39 (or variants thereof). For example, the aptamers of the invention comprise or consist of any one of SEQ ID NOs 36 or 38 (or variants thereof). As described further herein, these aptamers bind to the S1 subunit of the SARS-CoV-2 spike protein and also bind to the equivalent protein in SARS-CoV and/or MERS-CoV.

In certain embodiments, aptamers of the invention comprise or consist of a nucleic acid sequence having at least 85%, at least 90%, at least 95%, at least 96%, at least 97%, at least 98%, at least 99% or more sequence identity to the nucleotide sequence of any one of the sequences as described herein.

As used herein, “sequence identity” refers to the percentage nucleotide identity (or homology) over the entire length of the defined sequence (SEQ ID NO). The sequence identity is calculated based on the percentage of nucleotides in a candidate sequence that are identical with the nucleotides in said sequences after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, CLUSTALW or MegAlign (DNASTAR) software. For example, % nucleic acid sequence identity values can be generated using sequence comparison computer programs found on the European Bioinformatics Institute website (http://www.ebi.ac.uk).

With respect to any protein sequences, “sequence identity” also refers to the percentage amino acid identity (or homology) over the entire length of the defined sequence (SEQ ID NO). The sequence identify is calculated based on the percentage of amino acid residues in a sequence that are identical with the amino acid residues in said sequences after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLASTP.

In certain embodiments, aptamers of the invention comprise or consist of a minimal effective fragment of any one of the sequences as described herein. For example, the aptamers may comprise or consist of a minimal effect fragment of any one of:
SEQ ID NOs 1 to 53;
SEQ ID NOs 54 to 113;
SEQ ID NOs 1, 2, 4 to 6, 8 to 14, 16 to 26, 28 to 46 or 48 to 49;
SEQ ID NOs 4, 5, 6, 14, 17, 18, 21, 29, 32, 38, 45, 50;
SEQ ID NOs 1, 2, 4 to 6, 8 to 12, 14, 16 to 17, 19 to 25, 29, 31 to 32, 35 to 38, 43 to 45 or 48 to 49;
SEQ ID NOs 4, 5, 6, 14, 17, 21, 29, 32, 38 or 45;
SEQ ID NOs 2, 4, 6, 8, 9, 13, 16, 17, 18, 20, 21, 22, 23, 24, 26, 31, 34, 35, 40 to 46, 48 or 49;
SEQ ID NOs 4, 6, 17, 18, 21 or 45;
SEQ ID NOs 2, 4, 6, 8, 9, 16, 17, 20, 21, 22, 23, 24, 31, 35, 43 to 45, 48 or 49;
SEQ ID NOs 4, 6, 17, 21 or 45;
SEQ ID NOs 4, 8, 9 or 43;
SEQ ID NOs 4, 8 or 9;
SEQ ID NOs 1, 5, 10, 11, 12, 14, 19, 25, 28 to 30, 32, 33, 36 to 39;
SEQ ID NOs 36 or 38;
SEQ ID NOs 4, 8, 9, 16, 21, 23, 31, 43 or 48;
SEQ ID NOs 9, 21, 23, 24 or 31;
SEQ ID NOs 9, 21, 23 or 31; or
SEQ ID NOs 10, 20, 24 or 44.

In preferred embodiments, the aptamers may comprise or consist of a minimal effective fragment of any one or more of SEQ ID NOs 4, 8, 9 or 43.

In even more preferred embodiments, the aptamers may comprise or consist of a minimal effective fragment of any one or more of SEQ ID NOs 4, 8 or 9.

Herein, a “minimal effective fragment” is understood to mean a fragment (e.g., portion) of the full-length aptamer capable of binding to SARS-CoV-2 spike protein S1 (and/or the RBD subunit of S1) with at least the same (or improved) specificity and/or affinity as compared to the full-length aptamer. A minimal effective fragment may compete for binding to SARS-CoV-2 spike protein S1 with the full-length aptamer.

In certain embodiments, the aptamers of the invention comprise or consist of any one of SEQ ID NOs 129 to 144 (or variants thereof).

In certain embodiments, the aptamer may comprise or consist of one or more of SEQ ID NOs 129, 130, 131, 132, 133 or 134 (or variants thereof). As described further herein, these sequences correspond to minimal effective fragments of S1 Aptamer A3 (S1_A3, SEQ ID. NO: 4).

In certain embodiments, the aptamer may comprise or consist of one or more of SEQ ID NOs 142, 143 or 144 (or variants thereof). As described further herein, these sequences correspond to minimal effective fragments of S1 Aptamer A6 (S1_A6, SEQ ID. NO: 8).

In certain embodiments, the aptamer may comprise or consist of one or more of SEQ ID. NOs 135, 136, 137, 138, 139, 140 and 141 (or variants thereof). As described further herein, these sequences correspond to minimal effective fragments of S1 Aptamer A8 (S1_A8, SEQ ID. NO: 9).

In certain embodiments, the aptamer may comprise or consist of one or more of SEQ ID NOs 132, 134, 140, 142 or 144 (or variants thereof). As described further herein, these minimal effective fragments are advantageously capable of binding to SARS-CoV-2 spike protein present in a saliva sample.

In preferred embodiments, the aptamer may comprise or consist of SEQ ID NOs 134, 144 or 140 (or variants thereof). As described herein, these fragments are the shortest fragments which retain the ability to bind to their target (e.g., the S1 subunit of the SARS-CoV-2 spike protein). Aptly these fragments also function in an ELISA-like assay.

In preferred embodiments, the aptamer may comprise or consist of SEQ ID NO: 4, 8, 9, 134, 140 and/or 144 or variants thereof as described herein.

In even more preferred embodiments, the aptamer may comprise or consist of SEQ ID NO: 140 or variants thereof as described herein.

S2 Aptamers

In certain embodiments, the aptamers of the invention comprise or consist of a nucleic acid sequence selected from any one of SEQ ID NOs 145 to 176 (i.e., aptamers generated against the S2 subunit of the SARS-CoV-2 spike protein).

In certain embodiments, aptamers of the invention comprise or consist of a nucleic acid sequence having at least 85%, at least 90%, at least 95%, at least 96%, at least 97%, at least 98%, at least 99% or more sequence identity to the nucleotide sequence of any one of SEQ ID NOs 145 to 176. For example, the aptamers of the invention may comprise or consist of a nucleic acid sequence having at least 85%, at least 90%, at least 95%, at least 96%, at least 97%, at least 98%, at least 99% or more sequence identity to any one of SEQ ID NOs 146, 150 and 171 (or variants thereof).

In certain embodiments, aptamers of the invention comprise or consist of a minimal effective fragment of any one of SEQ ID NOs 145 to 176. As described above, a “minimal effective fragment” is understood to mean a fragment (e.g., portion) of the full-length aptamer capable of binding to SARS-CoV-2 spike protein S2 with at least the same (or improved) specificity and/or affinity as compared to the full-length aptamer. A minimal effective fragment may compete for binding to SARS-CoV-2 spike protein S2 with the full-length aptamer.

In certain embodiments, the aptamers of the invention may comprise or consist of a nucleic acid sequence comprising at least about 15, 20, 25, 30, 35, 40, 45, 50, 55, 60, 65, 70, 75 80 or more consecutive nucleotides of any one of the sequences described herein. In this context the term “about” typically means the referenced nucleotide sequence length plus or minus 10% of that referenced length.

In certain embodiments, aptamers of the invention comprise or consist of a nucleic acid sequence comprising at least about 15, 20, 25, 30, 35, 40, 45, 50, 55, 60 or more consecutive nucleotides of a sequence having at least 85%, at least 90%, at least 95%, at least 96%, at least 97%, at least 98%, at least 99% or more sequence identity with any of the sequences described herein.

In preferred embodiments, the aptamers of the invention comprise or consist of any one of SEQ ID NOs 146, 150 and 171 (or variants thereof). In a comparison between all the sequenced S2 binding aptamer clones, these aptamers represent the most prevalent sequences. These aptamers specifically bind with high affinity to the S2 subunit of the SARS-CoV-2 spike protein.

In preferred embodiments, the aptamers may comprise or consist of a minimal effect fragment of any one of SEQ ID NOs 146, 150 and 171 (or variants thereof). For example, the aptamers of the invention may comprise or consist of any one of SEQ ID NOs 177 to 192 (or variants thereof).

In certain embodiments, the aptamer may comprise or consist of any one of SEQ ID NOs 177 to 182 (or variants thereof). As described further herein, these sequences correspond to minimal effective fragments of S2 Aptamer S2 A2 (SEQ ID. NO: 146).

In preferred embodiments, the aptamer may comprise or consist of SEQ ID NO: 177 (or variants thereof). These aptamers specifically bind with high affinity to the S2 subunit of the SARS-CoV-2 spike protein.

In preferred embodiments, the aptamers of the invention comprise or consist of SEQ ID NO 179 (or variants thereof). These aptamers specifically bind to SARS-CoV-2 spike protein in its native trimeric form.

In certain embodiments, the aptamer may comprise or consist of any one of SEQ ID NOs 183 to 187 (or variants thereof). As described further herein, these sequences correspond to minimal effective fragments of S2 Aptamer S2_B1 (SEQ ID. NO: 150).

In preferred embodiments, the aptamer may comprise or consist of SEQ ID NO: 183 (or variants thereof). These aptamers specifically bind with high affinity to the S2 subunit of the SARS-CoV-2 spike protein.

In preferred embodiments, the aptamers of the invention comprise or consist of SEQ ID NO 188 (or variants thereof). As described further herein, these aptamers specifically bind to SARS-CoV-2 spike protein in its native trimeric form.

In certain embodiments, the aptamer may comprise or consist of one or more of SEQ ID NOs 189 to 192 (or variants thereof). As described further herein, these sequences correspond to minimal effective fragments of S2 Aptamer_S2_G1 (SEQ ID. NO: 171).

In preferred embodiments, the aptamer may comprise or consist of SEQ ID NO: 191. These aptamers specifically bind with high affinity to the S2 subunit of the SARS-CoV-2 spike protein.

In preferred embodiments, the aptamers of the invention comprise or consist of SEQ ID NO 190 (or variants thereof as described herein). As described further herein, these aptamers specifically bind to SARS-CoV-2 spike protein in its native trimeric form.

In preferred embodiments, the aptamer may comprise or consist of SEQ ID NO: 146, 150, 171, 179, 188 and/or 190 or variants thereof as described herein.

The aptamers of the invention may comprise natural or non-natural nucleotides and/or base derivatives (or combinations thereof). In certain embodiments, the aptamers comprise one or more modifications such that they comprise a chemical structure other than deoxyribose, ribose, phosphate, adenine (A), guanine (G), cytosine (C), thymine (T), or uracil (U). The aptamers may be modified at the nucleobase, at the sugar or at the phosphate backbone.

In certain embodiments, the aptamers comprise one or more modified nucleotides. Exemplary modifications include for example nucleotides comprising an alkylation, arylation or acetylation, alkoxylation, halogenation, amino group, or another functional group. Examples of modified nucleotides include 2′-fluoro ribonucleotides, 2′-NH₂—, 2′-OCH₃— and 2′-O-methoxyethyl ribonucleotides, which are used for RNA aptamers.

The aptamers of the invention may be wholly or partly phosphorothioate or DNA, phosphorodithioate or DNA, phosphoroselenoate or DNA, phosphorodiselenoate or DNA, locked nucleic acid (LNA), peptide nucleic acid (PNA), N3′-P5′phosphoramidate RNA/DNA, cyclohexene nucleic acid (CeNA), tricyclo DNA (tcDNA) or spiegelmer, or the phosphoramidate morpholine (PMO) components or any other modification known to those skilled in the art (see also Chan et al., Clinical and Experimental Pharmacology and Physiology (2006) 33, 533-540).

Some of the modifications allow the aptamers to be stabilized against nucleic acid-cleaving enzymes. In the stabilization of the aptamers, a distinction can generally be made between the subsequent modification of the aptamers and the selection with already modified RNA/DNA. The stabilization does not necessarily affect the affinity of the modified RNA/DNA aptamers but prevents the rapid decomposition of the aptamers in an organism or biological solutions by RNases/DNases. An aptamer is referred to as stabilized in the context of the present invention if the half-life in the sample (e.g., biological medium) is greater than one minute, preferably greater than one hour, more preferably greater than one day. The aptamers may also be modified with reporter molecules which, in addition to the detection of the labelled aptamers, may also contribute to increasing the stability.

Aptamers are characterised by the formation of a specific three-dimensional structure that depends on the nucleic acid sequence. The three-dimensional structure of an aptamer arises due to Watson and Crick intramolecular base pairing, Hoogsteen base pairing (quadruplex), wobble-pair formation or other non-canonical base interactions. This structure enables aptamers, analogous to antigen-antibody binding, to bind target structures accurately. A nucleic acid sequence of an aptamer may, under defined conditions, have a three-dimensional structure that is specific to a defined target structure.

The invention also provides aptamers that compete for binding to SARS-CoV-2 spike protein with aptamers as described herein. In certain embodiments, the invention provides aptamers that compete for binding to SARS-CoV-2 spike protein S1 (and/or the RBD of the S1 subunit) and/or S2 subunit with the aptamers set forth in any of the sequences described herein.

In certain embodiments, competition assays may be used to identify an aptamer that competes for binding to SARS-CoV-2 spike protein S1 and/or S2. In an exemplary competition assay, immobilized SARS-CoV-2 spike protein S1 and/or S2 is incubated in a solution comprising a first labelled aptamer that binds to the spike protein S1 and/or S2 and a second unlabelled aptamer that is being tested for its ability to compete with the first aptamer for binding to SARS-CoV-2 spike protein S1 and/or S2. As a control, immobilized SARS-CoV-2 spike protein S1 and/or S2 may be incubated in a solution comprising the first labelled aptamer but not the second unlabelled aptamer. After incubation under conditions permissive for binding of the first aptamer to SARS-CoV-2 spike protein S1 and/or S2 excess unbound aptamer may be removed, and the amount of label associated with immobilized SARS-CoV-2 spike protein S1 and/or S2 measured. If the amount of label associated with immobilized SARS-CoV-2 spike protein S1 and/or S2 is substantially reduced in the test sample relative to the control sample, then that indicates that the second aptamer is competing with the first aptamer for binding to SARS-CoV-2 spike protein S1 and/or S2.

Aptamer Pairs

In certain embodiments, the invention provides more than one aptamer, e.g., two, three, four, five or more aptamers.

In certain embodiments, the invention provides two or more aptamers that bind to two or more different regions of the SARS-CoV-2 spike protein as described herein (including in its trimeric form). By way of illustration only, a first aptamer may bind to the RBD within the S1 subunit and a second aptamer may bind to a different region of the S1 subunit. Alternatively, a first aptamer may bind to a first region within the RBD of the S1 subunit and a second aptamer may bind to a second different region within the RBD.

In certain embodiments, the invention provides two or more aptamers that bind to two or more different subunits of the SARS-CoV-2 spike protein as described herein. By way of illustration only, a first aptamer may bind to the S1 subunit and a second aptamer may bind to the S2 subunit. Aptamers capable of specifically binding to the S1 subunit or the S2 subunit are further described herein.

In certain embodiments, the invention provides two or more aptamers for use in a two-site binding assay (e.g., sandwich assay). Exemplary sandwich assays include, but are not limited to, ELISA, lateral flow assays and many other assays known to one skilled in the art. Such assays may utilise compatible pairs of aptamers capable of binding to different regions (e.g., epitopes) and/or subunits of the SARS-CoV-2 spike protein as described herein. In other words, the binding of one aptamer (of a compatible pair) to the SARS-CoV-2 spike protein may not interfere with (or affect) the binding of the second aptamer (of the compatible pair) to the equivalent protein.

In certain embodiments, the invention provides two or more aptamers (e.g., a compatible pair, triplet, or more) selected from any two or more nucleic acids selected from:
SEQ ID NOs 1 to 53 (or variants thereof);
SEQ ID NOs 54 to 113 (or variants thereof);
SEQ ID NOs 1, 2, 4 to 6, 8 to 14, 16 to 26, 28 to 46 or 48 to 49 (or variants thereof);
SEQ ID NOs 4, 5, 6, 14, 17, 18, 21, 29, 32, 38, 45, 50 (or variants thereof);
SEQ ID NOs 1, 2, 4 to 6, 8 to 12, 14, 16 to 17, 19 to 25, 29, 31 to 32, 35 to 38, 43 to 45 or 48 to 49 (or variants thereof);
SEQ ID NOs 4, 5, 6, 14, 17, 21, 29, 32, 38 or 45 (or variants thereof);
SEQ ID NOs 2, 4, 6, 8, 9, 13, 16, 17, 18, 20, 21, 22, 23, 24, 26, 31, 34, 35, 40 to 46, 48 or 49 (or variants thereof);
SEQ ID NOs 4, 6, 17, 18, 21 or 45 (or variants thereof);
SEQ ID NOs 2, 4, 6, 8, 9, 16, 17, 20, 21, 22, 23, 24, 31, 35, 43 to 45, 48 or 49 (or variants thereof);
SEQ ID NOs 4, 6, 17, 21 or 45 (or variants thereof);
SEQ ID NOs 4, 8, 9 or 43 (or variants thereof);
SEQ ID NOs 1, 5, 10, 11, 12, 14, 19, 25, 28 to 30, 32, 33, 36 to 39 (or variants thereof);
SEQ ID NOs 36 or 38 (or variants thereof);
SEQ ID NOs 4, 8, 9, 16, 21, 23, 31, 43 or 48 (or variants thereof);
SEQ ID NOs 9, 21, 23, 24 or 31 (or variants thereof);
SEQ ID NOs 9, 21, 23 or 31 (or variants thereof);
SEQ ID NOs 129, 130, 131, 132, 133, 134, 135, 136, 137, 138, 139, 140, 141, 142, 143 or 144 (or variants thereof); or
SEQ ID NOs 132, 134, 140, 142 or 144 (or variants thereof).

In certain embodiments, the invention provides two or more aptamers (e.g., a compatible pair, triplet, or more) selected from any two or more nucleic acids selected from any one of SEQ ID NOs 1 to 113 or 129 to 144 (or variants thereof) and any one of SEQ ID NOs 145 to 192.

In certain embodiments, the invention provides two or more aptamers selected from any one of SEQ ID NOs 1 to 113 or 129 to 144 (or variants thereof) and any one of SEQ ID NOs 145, 148, 150, 152 to 154, 156 to 159, 162 to 170, 172, 173, 175 or 177 to 192.

In preferred embodiments, the invention provides two or more aptamers selected from any one of:
SEQ ID NO 4 (or variants thereof) and SEQ ID NO 146 (or variants thereof);
SEQ ID NO 4 (or variants thereof) and SEQ ID NO 150 (or variants thereof);
SEQ ID NO 4 (or variants thereof) and SEQ ID NO 171 (or variants thereof);
SEQ ID NO 4 (or variants thereof) and SEQ ID NO 177 (or variants thereof);
SEQ ID NO 4 (or variants thereof) and SEQ ID NO 179 (or variants thereof);
SEQ ID NO 4 (or variants thereof) and SEQ ID NO 183 (or variants thereof);
SEQ ID NO 4 (or variants thereof) and SEQ ID NO 188 (or variants thereof);
SEQ ID NO 4 (or variants thereof) and SEQ ID NO 190 (or variants thereof);
SEQ ID NO 4 (or variants thereof) and SEQ ID NO 191 (or variants thereof)
SEQ ID NO 8 (or variants thereof) and SEQ ID NO 146 (or variants thereof);
SEQ ID NO 8 (or variants thereof) and SEQ ID NO 150 (or variants thereof);
SEQ ID NO 8 (or variants thereof) and SEQ ID NO 171 (or variants thereof);
SEQ ID NO 8 (or variants thereof) and SEQ ID NO 177 (or variants thereof);
SEQ ID NO 8 (or variants thereof) and SEQ ID NO 179 (or variants thereof);
SEQ ID NO 8 (or variants thereof) and SEQ ID NO 183 (or variants thereof);
SEQ ID NO 8 (or variants thereof) and SEQ ID NO 188 (or variants thereof);
SEQ ID NO 8 (or variants thereof) and SEQ ID NO 190 (or variants thereof);
SEQ ID NO 8 (or variants thereof) and SEQ ID NO 191 (or variants thereof);
SEQ ID NO 9 (or variants thereof) and SEQ ID NO 146 (or variants thereof);
SEQ ID NO 9 (or variants thereof) and SEQ ID NO 150 (or variants thereof);
SEQ ID NO 9 (or variants thereof) and SEQ ID NO 171 (or variants thereof);
SEQ ID NO 9 (or variants thereof) and SEQ ID NO 177 (or variants thereof);
SEQ ID NO 9 (or variants thereof) and SEQ ID NO 179 (or variants thereof);
SEQ ID NO 9 (or variants thereof) and SEQ ID NO 183 (or variants thereof);
SEQ ID NO 9 (or variants thereof) and SEQ ID NO 188 (or variants thereof);
SEQ ID NO 9 (or variants thereof) and SEQ ID NO 190 (or variants thereof);
SEQ ID NO 9 (or variants thereof) and SEQ ID NO 191 (or variants thereof);
SEQ ID NO 43 (or variants thereof) and SEQ ID NO 146 (or variants thereof);
SEQ ID NO 43 (or variants thereof) and SEQ ID NO 150 (or variants thereof);
SEQ ID NO 43 (or variants thereof) and SEQ ID NO 171 (or variants thereof);
SEQ ID NO 43 (or variants thereof) and SEQ ID NO 177 (or variants thereof);
SEQ ID NO 43 (or variants thereof) and SEQ ID NO 179 (or variants thereof);
SEQ ID NO 43 (or variants thereof) and SEQ ID NO 183 (or variants thereof);
SEQ ID NO 43 (or variants thereof) and SEQ ID NO 188 (or variants thereof);
SEQ ID NO 43 (or variants thereof) and SEQ ID NO 190 (or variants thereof);
SEQ ID NO 43 (or variants thereof) and SEQ ID NO 191 (or variants thereof);
SEQ ID NO 134 (or variants thereof) and SEQ ID NO 146 (or variants thereof);
SEQ ID NO 134 (or variants thereof) and SEQ ID NO 150 (or variants thereof);
SEQ ID NO 134 (or variants thereof) and SEQ ID NO 171 (or variants thereof);
SEQ ID NO 134 (or variants thereof) and SEQ ID NO 177 (or variants thereof);
SEQ ID NO 134 (or variants thereof) and SEQ ID NO 179 (or variants thereof);
SEQ ID NO 134 (or variants thereof) and SEQ ID NO 183 (or variants thereof);
SEQ ID NO 134 (or variants thereof) and SEQ ID NO 188 (or variants thereof);
SEQ ID NO 134 (or variants thereof) and SEQ ID NO 190 (or variants thereof);
SEQ ID NO 134 (or variants thereof) and SEQ ID NO 191 (or variants thereof);
SEQ ID NO 144 (or variants thereof) and SEQ ID NO 146 (or variants thereof);
SEQ ID NO 144 (or variants thereof) and SEQ ID NO 150 (or variants thereof);
SEQ ID NO 144 (or variants thereof) and SEQ ID NO 171 (or variants thereof);
SEQ ID NO 144 (or variants thereof) and SEQ ID NO 177 (or variants thereof);
SEQ ID NO 144 (or variants thereof) and SEQ ID NO 179 (or variants thereof);
SEQ ID NO 144 (or variants thereof) and SEQ ID NO 183 (or variants thereof);
SEQ ID NO 144 (or variants thereof) and SEQ ID NO 188 (or variants thereof);
SEQ ID NO 144 (or variants thereof) and SEQ ID NO 190 (or variants thereof);
SEQ ID NO 144 (or variants thereof) and SEQ ID NO 191 (or variants thereof);
SEQ ID NO 140 (or variants thereof) and SEQ ID NO 146 (or variants thereof);
SEQ ID NO 140 (or variants thereof) and SEQ ID NO 150 (or variants thereof);
SEQ ID NO 140 (or variants thereof) and SEQ ID NO 171 (or variants thereof);
SEQ ID NO 140 (or variants thereof) and SEQ ID NO 177 (or variants thereof);
SEQ ID NO 140 (or variants thereof) and SEQ ID NO 179 (or variants thereof);
SEQ ID NO 140 (or variants thereof) and SEQ ID NO 183 (or variants thereof);
SEQ ID NO 140 (or variants thereof) and SEQ ID NO 188 (or variants thereof);
SEQ ID NO 140 (or variants thereof) and SEQ ID NO 190 (or variants thereof);
SEQ ID NO 140 (or variants thereof) and SEQ ID NO 191 (or variants thereof);

In even more preferred embodiments, the invention provides at least two aptamers, wherein the aptamers are selected from SEQ ID NO: 140 (or variants thereof) and SEQ ID NO: 190 (or variants thereof).

As described herein, such aptamer pairs are particularly useful in sandwich assays or the like. In alternative embodiments (e.g., biosensor applications etc.) any one aptamer as described herein may be used.

Linker Sequence

In certain embodiments, the one or more aptamers of the invention further comprise one or more linker sequences. For example, the linker sequence may comprise a nucleic acid sequence which is configured to hybridise to at least a portion of the aptamer. The linker sequence may be configured to form a double-stranded duplex structure with at least a portion of the aptamer of the invention.

In certain embodiments, the linker sequence is between about 10 to about 20 nucleotides in length, e.g., about 10, 11, 12, 13, 14, 15, 16, 17, 18, 19, 20 or more nucleotides in length. Typically, the linker sequence is complementary to the aptamer over at least a portion of its length.

In certain embodiments, the linker sequence comprises a suitable functional moiety to allow surface attachment to the aptamer. The functional moiety may be selected from biotin, thiol, and amine or any other suitable group known to those skilled in the art.

In certain embodiments, the linker sequence or aptamer comprises a spacer molecule e.g., a spacer molecule selected from a polynucleotide molecule, C6 spacer molecule, a C12 spacer molecule, another length C spacer molecule, a hexaethylene glycol molecule, a hexanediol, and/or polyethylene glycol. The linker may be for example a biotin linker. In certain embodiments, the linker sequence or aptamer may be conjugated to streptavidin, avidin and/or neutravidin.

In certain embodiments, the linker sequence or aptamer may be modified for attachment to the support surface. For example, the linker sequence or aptamer may be attached via a silane linkage. The linker sequence or aptamer may be succinylated (e.g., to attach the linker sequence or aptamer to aminophenyl or aminopropyl-derivatized glass). Aptly, the support is aminophenyl or aminopropyl-derivatized. In certain embodiments, the linker sequence or aptamer comprises a NH₂ modification (e.g., to attach to epoxy silane or isothiocyanate coated glass). Typically, the support surface is coated with an epoxy silane or isothiocyanate. In certain embodiments, the linker sequence or aptamer is hydrazide-modified in order to attach to an aldehyde or epoxide molecule.

Support

In certain embodiments, the aptamer or linker sequence is attached to a support. Typically, the support is a solid support such as a wafer, slide, plate, membrane or a bead. The support may be a two-dimensional support e.g., a microplate or a three-dimensional support e.g., a bead. In certain embodiments, the support may comprise at least one or more silica wafers, fibres, magnetic beads etc. In certain embodiments, the solid support is part of a swab that is derivatised with one or more aptamers of the invention. In preferred embodiments, the solid support is part of a lateral flow device that is derivatised with one or more aptamers of the invention.

In certain embodiments, the support may comprise at least one nanoparticle e.g., gold nanoparticles or the like. In yet further embodiments, the support comprises a microtiter or other assay plate, a strip, a membrane, a film, a gel, a chip, a microparticle, a nanofiber, a nanotube, a micelle, a micropore, a nanopore, a silica-fibre or similar substrate forming a swab or a biosensor surface. In certain embodiments, the biosensor surface may be a probe tip surface, a biosensor flow-channel or similar.

In certain embodiments, the aptamer or linker sequence may be attached, directly or indirectly, to a magnetic bead, which may be e.g., carboxy-terminated, avidin-modified or epoxy-activated or otherwise modified with a compatible reactive group.

Immobilisation of oligonucleotides to a support e.g., a solid phase support can be accomplished in a variety of ways and in any manner known to those skilled in the art for immobilising DNA or RNA on solids. The immobilisation of aptamers on nanoparticles is e.g., as described in WO2005/13817. For example, a solid phase of paper or a porous material may be wetted with the liquid phase aptamer, and the liquid phase subsequently volatilized leaving the aptamer in the paper or porous material.

In certain embodiments, the support comprises a membrane, e.g., a nitrocellulose, a polyethylene (PE), a polytetrafluoroethylene (PTFE), a polypropylene (PP), a cellulose acetate (CA), a polyacrylonitrile (PAN), a polyimide (PI), a polysulf one (PS), a polyethersulfone (PES) membrane or an inorganic membrane comprising aluminium oxide (Al₂O₃), silicon oxide (SiO₂) and/or zirconium oxide (ZrO₂). Particularly suitable materials from which a support can be made include for example inorganic polymers, organic polymers, glasses, organic and inorganic crystals, minerals, oxides, ceramics, metals, especially precious metals, carbon and semiconductors. A particularly suitable organic polymer is a polymer based on polystyrene. Biopolymers, such as cellulose, dextran, agar, agarose and Sephadex, which may be functionalized, in particular as nitrocellulose or cyanogen bromide Sephadex, can be used as polymers which provide a solid support.

Detectable Labels

In certain embodiments, the aptamers of the invention are used to detect and/or quantify the amount of SARS-CoV-2 in a sample. Typically, the aptamers comprise a detectable label. Any label capable of facilitating detection and/or quantification of the aptamers may be used herein.

In certain embodiments, the detectable label is a fluorescent moiety, e.g., a fluorescent/quencher compound. Fluorescent/quencher compounds are known in the art. See, for example, Mary Katherine Johansson, Methods in Molecular Biol. 335: Fluorescent Energy Transfer Nucleic Acid Probes: Designs and Protocols, 2006, Didenko, ed., Humana Press, Totowa, N.J., and Marras et al., 2002, Nucl. Acids Res. 30, e122 (incorporated by reference herein).

In certain embodiments, the detectable label is FAM. In certain embodiments, the FAM-label is situated at the first or second primer region of the aptamer. The person skilled in the art would understand that the label could be located at any suitable position within the aptamer. Moieties that result in an increase in detectable signal when in proximity of each other may also be used herein, for example, as a result of fluorescence resonance energy transfer (“FRET”); suitable pairs include but are not limited to fluoroscein and tetramethylrhodamine; rhodamine 6G and malachite green, and FITC and thiosemicarbazole, to name a few.

In certain embodiments, the detectable label is selected from a fluorophore, a nanoparticle, a quantum dot, an enzyme, a radioactive isotope, a pre-defined sequence portion, a biotin, a desthiobiotin, a thiol group, an amine group, an azide, an aminoallyl group, a digoxigenin, an antibody, a catalyst, a colloidal metallic particle, a colloidal non-metallic particle, an organic polymer, a latex particle, a nanofiber, a nanotube, a dendrimer, a protein, and a liposome.

In certain embodiments, the detectable label is a fluorescent protein such as Green Fluorescent Protein (GFP) or any other fluorescent protein known to those skilled in the art.

In certain embodiments, the detectable label is an enzyme. For example, the enzyme may be selected from horseradish peroxidase, alkaline phosphatase, urease, β-galactosidase or any other enzyme known to those skilled in the art.

In certain embodiments, the nature of the detection will be dependent on the detectable label used. For example, the label may be detectable by virtue of its colour e.g., gold nanoparticles. A colour can be detected quantitatively by an optical reader or camera e.g., a camera with imaging software.

In certain embodiments, the detectable label is a fluorescent label e.g., a quantum dot. In such embodiments, the detection means may comprise a fluorescent plate reader, strip reader or similar, which is configured to record fluorescence intensity.

In embodiments in which the detectable label is an enzyme label, the detection means may, for example, be colorimetric, chemiluminescence and/or electrochemical (for example, using an electrochemical detector). Typically, electrochemical sensing is through conjugation of a redox reporter (e.g. methylene blue or ferrocene) to one end of the aptamer and a sensor surface to the other end. Typically, a change in aptamer conformation upon target binding changes the distance between the reporter and sensor to provide a readout.

In certain embodiments, the detectable label may further comprise enzymes such as horseradish peroxidase (HRP), Alkaline phosphatase (APP) or similar, to catalytically turnover a substrate to give an amplified signal.

In certain embodiments, the invention provides a complex (e.g., conjugate) comprising aptamers of the invention and a detectable molecule. Typically, the aptamers of the invention are covalently or physically conjugated to a detectable molecule.

In certain embodiments, the detectable molecule is a visual, optical, photonic, electronic, acoustic, opto-acoustic, mass, electrochemical, electro-optical, spectrometric, enzymatic, or otherwise physically, chemically or biochemically detectable label.

In certain embodiments, the detectable molecule is detected by luminescence, UV/VIS spectroscopy, enzymatically, electrochemically or radioactively. Luminescence refers to the emission of light. For example, photoluminescence, chemiluminescence and bioluminescence are used for detection of the label. In photoluminescence or fluorescence, excitation occurs by absorption of photons. Exemplary fluorophores include, without limitation, bisbenzimidazole, fluorescein, acridine orange, Cy5, Cy3 or propidium iodide, tetramethyl-6-carboxyhodamine (TAMRA), Texas Red (TR), rhodamine, Alexa Fluor dyes (et al. Fluorescent dyes of different wavelengths from different companies), quantum dots or other related semiconductor particles; which can be covalently coupled to aptamers.

In certain embodiments, the detectable molecule is a non-fluorescent particle e.g., gold nanoparticle, colloidal non-metallic particle, organic polymer, latex particle, nanofiber (e.g., carbon nanofiber), nanotube (e.g., carbon nanotube), dendrimer, protein or liposome with signal-generating substances. Colloidal particles can be detected colorimetrically.

In certain embodiments, the detectable molecule is an enzyme. In certain embodiments, the enzyme may convert substrates to coloured products, e.g., peroxidase, luciferase, β-galactosidase or alkaline phosphatase. For example, the colourless substrate X-gal is converted by the activity of β-galactosidase to a blue product whose colour is visually detected.

In certain embodiments, the detection molecule is a radioactive isotope. The detection can also be carried out by means of radioactive isotopes with which the aptamer is labelled, including but not limited to 3H, 14C, 32P, 33P, 35S or 125I, more preferably 32P, 33P or 125I. In the scintillation counting, the radioactive radiation emitted by the radioactively labelled aptamer target complex is measured indirectly. A scintillator substance is excited by the isotope's radioactive emissions. During the transition of the scintillation material, back to the ground state, the excitation energy is released again as flashes of light, which are amplified and counted by a photomultiplier.

In certain embodiments, the detectable molecule is selected from digoxigenin and biotin. Thus, the aptamers may also be labelled with digoxigenin or biotin, which are bound for example by antibodies or streptavidin, which may in turn carry a label, such as an enzyme conjugate. The prior covalent linkage (conjugation) of an aptamer with an enzyme can be accomplished in several known ways. Detection of aptamer binding may also be achieved through labelling of the aptamer with a radioisotope in an RIA (radioactive immunoassay), preferably with 125I, or by fluorescence in a FIA (fluoroimmunoassay) with fluorophores, preferably with fluorescein or FITC.

Apparatus

The apparatus according to the invention may be provided in a number of different formats. In certain embodiments, the invention provides apparatus for detecting the presence, absence or level of SARS-CoV-2 in a sample, the apparatus comprising an aptamer as described herein.

In certain embodiments, the apparatus comprises a support as described herein. For example, in the absence of SARS-CoV-2, the aptamer may be secured directly or indirectly to a support to immobilise it.

In certain embodiments, the apparatus comprises a linker sequence as described herein.

In certain embodiments, the aptamer of the invention is attached directly or indirectly (e.g. via a linker) to the support surface. For example, the aptamers may be immobilised through a linker which is chemically attached to the end of the aptamer to allow direct immobilisation of the aptamer.

In certain embodiments, the linker sequence is a DNA or an RNA molecule or a mixed DNA/RNA molecule, wherein optionally the linker molecule comprises one or more modified nucleotides.

In certain embodiments, the apparatus may be a biosensor. Biosensors are found in many different formats. In certain embodiments, the biosensor comprises the aptamer and a transducer which converts the binding event between the aptamer and SARS-CoV-2 spike protein S1 and/or S2 into an electrically quantifiable signal. The biosensor may be comprised in a vessel or a probe or the like.

In addition, the apparatus may further comprise other elements such as a signal processing device, output electronics, a display device, a data processing device, a data memory device and interfaces to other devices. In certain embodiments, a sample containing SARS-CoV-2 is brought into contact with the biosensor. SARS-CoV-2 may then be identified via the changes in the aptamer properties upon specific binding of SARS-CoV-2 spike protein S1 and/or S2 to the aptamer.

The sensitivity of the sensor may be influenced by the transducer used. The transducer converts the signal from the binding event, which is proportional to the concentration of the target molecule in the sample, into an electrically quantifiable measurement signal. Signalling occurs due to the molecular interaction between the aptamer and SARS-CoV-2. With a biosensor according to the invention, qualitative, quantitative and/or semi-quantitative analytical information can be obtained.

The measurement in optical transducers can be based on principles of photometry, whereby, for example, colour or luminescence intensity changes are detected. Optical methods include the measurement of fluorescence, phosphorescence, bioluminescence and chemiluminescence, infrared transitions and light scattering. The optical methods also include the measurement of layer thickness changes when SARS-CoV-2 spike protein S1 is bound to the aptamer. The layer thickness can be measured, for example, by surface plasmon resonance (SPR), reflectometric interference spectroscopy (RIfS), biolayer interferometry (BLI) or similar.

Furthermore, the interference on thin layers (SPR or RIfS) and the change of the evanescent field can be measured. Acoustic transducers use the frequency changes of a piezoelectric quartz crystal, which detects highly sensitive mass changes that occur when target binds to aptamer. The quartz crystal used is placed in an oscillating electric field and the resonant frequency of the crystal is measured. A mass change on the surface of the quartz crystal can be quantified.

In certain embodiments, the apparatus is a BLI (Biolayer Interferometry) apparatus or similar apparatus. BLI is a label-free technology for measuring biomolecular interactions. It is an optical analytical technique that analyses changes in the interference pattern of white light reflected from two surfaces: a layer of immobilised ligand on the biosensor tip, and an internal reference layer. Any change in the number of molecules bound to the biosensor tip causes a shift in the interference pattern that can be measured in real-time. Only molecules binding to or dissociating from the biosensor can shift the interference pattern and generate a response profile on the BLI sensor. Unbound molecules, changes in the refractive index of the surrounding medium, or changes in flow rate do not affect the interference pattern.

In certain embodiments, the apparatus may comprise a nanopore detecting platform (e.g. Resistive Pulse Sensing (RPS) or the like). Typically, such detection platforms allow the detection of any aptamer of the invention via the use of complementary DNA hairpins. For example, any aptamer moving through the pore may be affected by protein binding thus providing an indirect measure of protein binding.

Some assay formats (such as electrochemical sensors) rely on a change in the local environment around the sensor surface to give a quantifiable readout. Some sensors simply detect the change in environment when the target (in this case SARS-CoV-2) binds to the surface immobilised ligand (an Aptamer or Optimer). Some sensors include a functional group on the immobilised ligand e.g., a redox reporter such as methylene blue, ferrocene or a nanoparticle to improve the signal.

Many aptamers undergo a conformational change upon target binding. This can alter the distance between the redox reporter and the sensor surface, leading to an increase in this response. Comparison of aptamer sequences show that some of the aptamers described herein form a tight structure which may be less likely to undergo a significant structural rearrangement upon target binding. Other aptamer sequences are less likely to form this strong structure, so may be more amenable to structural rearrangements.

In certain embodiments, the invention provides a functionalised electrode or biosensor surface comprising at least one or more of SEQ ID NO: 10, 20, 24 or 44. Without being bound by theory, it is understood these sequences are less likely to form G-quartet structures as compared to other aptamer sequences described herein.

Several aptamers from this structural class (SEQ ID NO: 10, 20, 24 or 44) were prepared with a thiol group on one end to allow immobilisation onto the gold sensor electrode, and a methylene blue redox report on the other end to allow detection. Both orientations of labelling (5′ thiol with 3′ methylene blue, and 5′ methylene blue with 3′ thiol) have been evaluated. Advantageously, the aptamers from this structural class are particularly effective in electrochemical sensor formats.

In preferred embodiments, the invention therefore provides an aptamer comprising any one or more of SEQ ID NO: 10, 20, 24 or 44 (or variants thereof as described elsewhere herein) wherein the aptamer is immobilised onto the (gold) surface of the electrode.

The invention also provides a test strip and/or lateral flow device comprising any aptamer or complex as described herein. Lateral flow devices may also be referred to as lateral flow tests, lateral flow assays and lateral flow immunoassays.

In certain embodiments, the lateral flow device comprises a support onto which a linker sequence is attached. The linker sequence may be configured to hybridise to at least a portion of an aptamer as described herein. Any sample as described herein (e.g., a swab, blood, sputum or plasma sample) may be introduced. If the sample comprises SARS-CoV-2, the aptamer may bind to the SARS-CoV-2 spike protein and undergo a conformational change, resulting in the aptamer disassociating from the linker sequence. Typically, however, the lateral flow device comprises a traditional LFD format in which the SARS-CoV-2 is ‘captured’ on a ‘test line’ compromising of an immobilised aptamer; followed by the detection with a second aptamer (which may be the same as the first aptamer) or antibody, which is conjugated to an appropriate detection molecule (e.g., latex bead, gold nanoparticles, fluorophore or similar).

In certain embodiments, the apparatus may be suitable for use in assays such as ELISA (enzyme-linked immunosorbent assay) and variants of which are known to those skilled in the art. When aptamers are used in place of antibodies, the resulting assay is often referred to as an “ELONA” (enzyme-linked oligonucleotide assay), “ELASA” (enzyme linked aptamer sorbent assay), “ELAA” (enzyme-linked aptamer assay) or similar. Incorporating aptamers into these ELISA-like assay platforms can result in increased sensitivity, allow a greater number of analytes to be detected; including analytes for which there are no antibodies available and a wide range of outputs, since aptamers can be conjugated to multiple reporter molecules including fluorophores, quencher molecules and/or any other detection moiety as described herein.

In preferred embodiments, the invention provides a lateral low device comprising (1) a first aptamer comprising a nucleic sequence selected from any one of SEQ ID NOs: 4, 8, 9, 43, 134, 140 or 144 (or any variant thereof as described herein) and (2) (i) a second aptamer comprising a nucleic sequence selected from any one of SEQ ID NOs: 4, 8, 9, 43, 134, 140 or 144 (or any variant thereof as described herein) wherein the second aptamer is different than the first aptamer or (ii) a second aptamer comprising a nucleic sequence selected from any one of SEQ ID NOs: 146, 150, 171, 177, 179, 183, 188, 190 or 191 (or any variant thereof as described herein). As described herein, such aptamer pairs are particularly effective in ELISA-like formats and/or lateral flow devices.

In even more preferred embodiments, the aptamer pair comprises SEQ ID NO: 140 (or any variant thereof as described herein) and SEQ ID NO: 190 (or any variant thereof as described herein). The aptamer set forth in SEQ ID NO: 140 (or variant thereof) may be bound to a solid support (e.g., test strip) and SEQ ID NO: 190 (or variant thereof) may be conjugated to a detectable label. Alternatively, SEQ ID NO: 190 (or variant thereof) may be bound to a solid support (e.g., test strip) and SEQ ID NO: 140 (or variant thereof) may be conjugated to a detectable label.

In certain embodiments, the apparatus may comprise a vessel. The aptamer of the invention may be immobilized via hybridization to a linker sequence in the vessel (e.g., the surface of the vessel). Alternatively, the aptamer of the invention may be directly immobilised on the surface of the vessel via a linker or functional group on the end of the aptamer.

Methods of Detecting SARS-CoV-2

In certain embodiments, the invention provides methods for detecting the presence, absence or amount of SARS-CoV-2 in a sample.

Any appropriate sample may be used. In addition, samples may be obtained using any relevant approach or technique known in in the art.

In certain embodiments, the sample is biological. For example, the sample may comprise whole blood, leukocytes, peripheral blood mononuclear cells, plasma, serum, sputum, breath, urine, semen, saliva, meningeal fluid, amniotic fluid, glandular fluid, lymph fluid, nipple aspirate, bronchial aspirate, synovial fluid, joint aspirate, cells, a cellular extract, stool, tissue, a tissue biopsy or cerebrospinal fluid.

In certain embodiments, the sample is a blood (e.g., plasma) sample. Alternatively, the sample may be a saliva or sputum (mucus or phlegm) sample. The sample may be pre-treated, such as by mixing, addition of enzymes, buffers, salt solutions or markers, or purified.

In certain embodiments, the sample is an unobtrusive breath sample. For example, the breath sample may not require obtrusive collection as compared to a swab sample. Typically, the unobtrusive breath sample is obtained using a standard breathalyser.

In certain embodiments, the sample is a faecal sample. For example, the sample may represent a pool of faecal samples from a region being monitored for an outbreak of COVID-19 infection within the population.

In certain embodiments, the sample is obtained by taking a swab test (e.g., throat and/or nose swab including, for example, nasopharyngeal, anterior nasal and/or anterior nares swabs). For example, a sample may comprise at least two different regions from the same subject (e.g., back of throat and inside of nose). Alternatively, the sample may be a blood or urine sample.

In certain embodiments, the sample is obtained from a subject having, or suspected of having, a COVID-19 infection. Typically, the subject is human. Typically, the subject has or is suspected of having a SARS-CoV-2 infection; symptoms include, but are not limited to, dry cough, shortness of breath or difficulty breathing, fever, chills, muscle and/or joint pain, headache/dizziness, sore throat and loss of taste or smell. In certain embodiments, the sample is obtained from a subject who is asymptomatic but who may later be at risk of developing (and/or spreading) the infection. In certain embodiments, the subject has been identified as being susceptible to a COVID-19 infection. In certain embodiments, the subject has been identified as at risk of developing severe symptoms of the disease.

In the methods for detecting the presence, absence, or amount of SARS-CoV-2 in a sample, the sample may be interacted (i.e., contacted) with any aptamer as described herein. For example, the sample and aptamers as described herein may be incubated under conditions sufficient for at least a portion of the aptamer to bind to any SARS-CoV-2 spike protein (e.g., the S1 and/or S2 subunit of the SARS-CoV-2 spike protein) in the sample.

Any suitable conditions for binding between the aptamers as described herein and the SARS-CoV-2 spike protein (e.g., the S1 and/or S2 subunit of the SARS-CoV-2 spike protein) may be used. In certain embodiments, the sample and aptamer are incubated at temperatures between about 20° C. and about 37° C. In certain embodiments, the sample and aptamer may be diluted to different concentrations (e.g., at least about 1%, 5%, 10%, 20%, 25%, 30%, 40%, 50%, 60%, 70% 80% v/v or more) with appropriate buffers (e.g., PBS or other buffer composition known to those skilled in the art). In certain embodiments, the sample and aptamer may be incubated whilst shaking and/or mixing. In certain embodiments, the sample and aptamer are incubated for at least 1 minute, at least 5 minutes, at least 15 minutes, at least 1 hour or more.

In certain embodiments, binding of the aptamer and SARS-CoV-2 spike protein S1 and/or S2 leads to formation of an aptamer-SARS-CoV-2 spike protein S1 and/or S2 complex. The binding or binding event may be detected, for example, visually, optically, photonically, electronically, acoustically, opto-acoustically, by mass, electrochemically, electro-optically, spectrometrically, enzymatically or otherwise chemically, biochemically or physically as described herein.

The binding of aptamer and SARS-CoV-2 spike protein (e.g., the S1 and/or S2 subunit of the SARS-CoV-2 spike protein) may be detected using any suitable technique. As discussed above, for example, binding of the aptamer and SARS-CoV-2 spike protein (e.g., the S1 and/or S2 subunit of the SARS-CoV-2 spike protein) may be detected using a biosensor. In certain embodiments, binding of the aptamer and SARS-CoV-2 spike protein (e.g., the S1 and/or S2 subunit of the SARS-CoV-2 spike protein) is detected using SPR, RIfS, BLI, LFD or ELONA as described herein, or other technique known to those skilled in the art.

Advantageously, the aptamers of the invention allow detection of clinically pathological amounts of SARS-CoV-2 spike protein S1 and/or S2. Typically, the aptamers of the invention have a detection limit of less than about 10000 pg/ml SARS-CoV-2, e.g. less than about 9000 pg/ml, less than about 8000 pg/ml, less than about 7000 pg/ml, less than about 6000 pg/ml, less than about 5000 pg/ml, less than about 4000 pg/ml, less than about 3000 pg/ml, less than about 2000 pg/ml, less than about 1000 pg/ml, less than about 900 pg/ml, less than about 800 pg/ml, less than about 700 pg/ml, less than about 600 pg/ml, less than about 500 pg/ml, less than about 400 pg/ml, less than about 300 pg/ml, less than about 200 pg/ml, less than about 100 pg/ml or less than about 50 pg/ml SARS-CoV-2. Typically, the aptamers of the invention have a detection range from about 50 pg/ml to about 10000 pg/ml SARS-CoV-2, e.g., from about 50 pg/ml to about 5000 pg/ml SARS-CoV-2. Thus, the aptamers are capable of binding to SARS-CoV-2 with high specificity and/or affinity and allow pathological and/or infectious ranges of active SARS-CoV-2 to be detected in a sample.

Advantageously, the aptamers of the invention allow pathological and/or infectious ranges of active SARS-CoV-2 to be detected in a sample in, for example, less than about 60 minutes, less than about 50 minutes. less than about 40 minutes, less than about 30 minutes, less than about 20 minutes, less than about 10 minutes, or less.

Methods of Enriching, Separating and/or Isolating SARS-CoV-2

In certain embodiments, the invention provides a method of enriching (e.g., concentrating), separating and/or isolating SARS-CoV-2 in a sample as described herein.

Typically, the invention provides a method of contacting an aptamer, complex, biosensor, test strip or apparatus as described herein with a sample as described herein. The binding of the aptamer with any SARS-CoV-2 spike protein S1 and/or S2 in the sample may then allow the virus in the sample to be enriched, separated and/or isolated prior to any subsequent treatment.

In certain embodiments, the aptamers of the invention are used to bind and concentrate SARS-CoV-2 virus in a sample. The resulting concentrate may then be used to improve the sensitivity of any COVID19 diagnostic method, for example qRT-PCR tests or the like.

In certain embodiments, the aptamers of the invention allow the amount of virus in a sample to be concentrated by at least 5×, 10×, 100×, 1000× or more prior to any technique to detect and/or quantify the virus in the initial sample.

In certain embodiments the aptamers of the invention are immobilised (e.g., in a nasal or oral swab) to capture more of the virus from respective samples. In further embodiments, the aptamers are immobilised on the inside (or surface) of a capillary, tube, fibre, membrane, plate or bead. Aptly the aptamer immobilised on the surface can then be mixed with the patient sample to ‘capture’ the virus. For example, the beads could then be concentrated, and the virus and/or viral genome released from the beads in a more concentrated sample.

In certain embodiments the aptamers of the invention may act as an affinity purification reagent, e.g., in affinity chromatography. In such embodiments, the aptamer is used to capture, purify and enrich the target of interest (the virus) but not any other non-target materials. The amount of the target (virus) in the eluted material may be purer and/or more concentrated compared to the initial sample.

In certain embodiments, the invention provides use of any aptamer, complex, biosensor, test strip or apparatus as described herein for detecting, enriching, separating and/or isolating SARS CoV-2 as described herein.

Therapeutic Aptamers

In certain embodiments, the aptamers of the invention are capable of inhibiting the interaction between the Receptor Binding Domain (RBD) within the S1 subunit of the SARS-CoV-2 spike protein (including its native trimeric form) and the Angiotensin-Converting Enzyme 2 (ACE2) receptor on the host cell surface. As such, the aptamers of the invention may act to limit virus-host cell entry and subsequent viral infection.

Aptamers may be tested for their ability to inhibit interactions between spike protein and the ACE2 cell surface receptor using any suitable technique. Such techniques include, for example, ligand binding assays such as radioactive ligand binding assays, saturation binding assays, competition binding assays, fluorescence polarization (FP) assay, fluorescence resonance energy transfer (FRET) assays, surface plasmon resonance (SPR) assays, Biolayer Interferometry (BLI), liquid phase ligand binding assays, immunoprecipitation assays, solid-phase ligand binding assays, multiwell plate assays, on-bead ligand binding assays, on-column ligand binding assays, filter assays, real-time cell-binding assays and the like.

The binding of the aptamer to SARS-CoV-2 spike protein S1 may inhibit the activity of the target antigen without provoking any undesirable side-effects. Furthermore, aptamers function well in physiological conditions, and have a high shelf-life, while still chemically synthesized in just a few minutes and in a cost-effective manner. Furthermore, due to their small size, aptamers can penetrate membranes and target antigens of smaller sizes.

In certain embodiments, the invention further provides aptamers against the S1 subunit of the SARS-CoV-2 spike protein that are capable of treating and/or alleviating the symptoms of COVID-19 infection.

In certain embodiments, the invention provides one or more therapeutic aptamers against the S1 subunit of the SARS-CoV-2 spike protein comprising a nucleic acid sequence as set forth in any one of the sequences as described herein.

In certain embodiments, the invention provides one or more therapeutic aptamers that bind to the S1 subunit of the spike protein from several related coronaviruses, rather than specifically binding to SARS-CoV-2 spike protein alone. For example, the therapeutic aptamer may be capable of cross-reacting with the S1 subunit of the spike proteins from other coronaviruses (e.g., SARS and/or MERS). Advantageously, such aptamers may be capable of treating multiple coronavirus infections.

In certain embodiments, the therapeutic aptamers of the invention may comprise or consist of the nucleic acid sequence as set forth in SEQ ID NOs 1, 5, 10, 11, 12, 14, 19, 25, 28 to 30, 32, 33, 36 to 39 (or variants thereof). For example, the therapeutic aptamer of the invention may comprise or consist of SEQ ID NO 36 or 38 (or variants thereof).

In certain embodiments, the invention provides one or more therapeutic aptamers that are capable of specifically binding to the S1 subunit of the SARS-CoV-2 spike protein. For example, the one or more therapeutic aptamers may specifically bind to the RBD of SARS-CoV-2 spike protein. In other words, the therapeutic aptamers may not cross-react with spike proteins from other coronaviruses (e.g., SARS and/or MERS). Advantageously, such aptamers may be capable of specifically treating COVID-19.

In certain embodiments, the therapeutic aptamers of the invention may comprise or consist of a nucleic acid sequence as set forth in any one of SEQ ID NOs 54 to 113. In certain embodiments, the therapeutic aptamers may comprise or consist of one or more nucleic acid sequences as set forth in any one of:
SEQ ID NOs 1, 2, 4 to 6, 8 to 14, 16 to 26, 28 to 46 or 48 to 49 (or variants thereof);
SEQ ID NOs 4, 5, 6, 14, 17, 18, 21, 29, 32, 38, 45, 50 (or variants thereof);
SEQ ID NOs 1, 2, 4 to 6, 8 to 12, 14, 16 to 17, 19 to 25, 29, 31 to 32, 35 to 38, 43 to 45 or 48 to 49 (or variants thereof);
SEQ ID NOs 4, 5, 6, 14, 17, 21, 29, 32, 38 or 45 (or variants thereof);
SEQ ID NOs 2, 4, 6, 8, 9, 13, 16, 17, 18, 20, 21, 22, 23, 24, 26, 31, 34, 35, 40 to 46, 48 or 49 (or variants thereof);
SEQ ID NOs 4, 6, 17, 18, 21 or 45 (or variants thereof);
SEQ ID NOs 2, 4, 6, 8, 9, 16, 17, 20, 21, 22, 23, 24, 31, 35, 43 to 45, 48 or 49 (or variants thereof);
SEQ ID NOs 4, 6, 17, 21 or 45 (or variants thereof);
SEQ ID NOs 4, 8, 9 or 43 (or variants thereof);
SEQ ID NOs 4, 8, 9, 16, 21, 23, 31, 43 or 48 (or variants thereof);
SEQ ID NOs 9, 21, 23, 24 or 31 (or variants thereof);
SEQ ID NOs 9, 21, 23 or 31 (or variants thereof);
SEQ ID NOs 129, 130, 131, 132, 133, 134, 135, 136, 137, 138, 139, 140, 141, 142, 143 or 144 (or variants thereof); or
SEQ ID NOs 132, 134, 140, 142 or 144 (or variants thereof).

In certain embodiments, the one or more aptamers against the S1 subunit of the SARS-CoV-2 spike protein of the invention are for use as a medicament.

In certain embodiments, the one or more aptamers against the S1 subunit of the SARS-CoV-2 spike protein of the invention are for use as a vaccine.

In certain embodiments, the one or more aptamers against the S1 subunit of the SARS-CoV-2 spike protein of the invention are conjugated to one or more therapeutic agents.

In certain embodiments, the one or more aptamers against the S1 subunit of the SARS-CoV-2 spike protein of the invention are used to help deliver another agent (e.g., anti-viral) to the COVID-19 virus.

In certain embodiments, the one or more aptamers against the S1 subunit of the SARS-CoV-2 spike protein of the invention are for use in the treatment and/or prevention of any disease or condition in which SARS-CoV-2 is implicated. Such diseases and/or conditions include, but are not limited to, acute respiratory distress syndrome, pneumonia, acute cardiac injury, dry cough, shortness of breath or difficulty breathing, fever, chills, muscle and/or joint pain, headache/dizziness, sore throat and/or loss of taste or smell.

In certain embodiments, the invention provides a method of administering a therapeutically effective amount of the one or more aptamers against the S1 subunit of the SARS-CoV-2 spike protein to a patient.

A “patient” includes any human or other mammalian subject that receives either prophylactic or therapeutic treatment. Treatment is considered prophylactic if administered to an individual susceptible to, or otherwise at risk of SARS-CoV-2 infection. Treatment is considered therapeutic if administered to an individual suspected of having, or already suffering from a disease and/or symptom associated with SARS-CoV-2 infection, such as COVID-19.

The patient may be asymptomatic. Alternatively, the patient may show symptoms of COVID-19 infection as described herein.

A “therapeutically effective amount” is an amount of an aptamer or composition (e.g., a pharmaceutical composition or aptamer-drug conjugate as also described herein) that produces a desired therapeutic effect in the patient, such as preventing or treating a target condition or alleviating symptoms associated with the condition. The precise therapeutically effective amount is an amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. A person skilled in the clinical and pharmacological arts will be able to determine a therapeutically effective amount of the aptamer to administer through routine experimentation, namely by monitoring a patient's response to administration of a compound and adjusting the dosage accordingly. For additional guidance, see Remington: The Science and Practice of Pharmacy 21st Edition, Univ. of Sciences in Philadelphia (USIP), Lippincott Williams & Wilkins, Philadelphia, Pa., 2005.

In certain embodiments, the invention provides a pharmaceutical composition comprising the aptamer against the S1 subunit of the SARS-CoV-2 spike protein of the invention. For example, in certain embodiments the invention provides a pharmaceutical composition comprising an aptamer against the S1 subunit of the SARS-CoV-2 spike protein of the invention and at least one pharmaceutically acceptable excipient, adjuvant or carrier.

Preparation of pharmaceutical compositions will be known to those skilled in the art. Typically, such compositions may be prepared as injectables, as tablets or other solids for oral administration (including time release capsules); or in any other form currently used in the art, including, but not limited to, eye drops, creams, lotions, salves, inhalants and the like.

Furthermore, injectables can be prepared either as liquid solutions (or suspensions) or solid forms suitable for solution in (or suspension in) liquid prior to injection.

The aptamer or pharmaceutical compositions against the S1 subunit of the SARS-CoV-2 spike protein described herein can be administered in a continuous or intermittent regime. For example, the regime may include multiple administrations of the therapeutic agent over a period of time.

The aptamer or pharmaceutical compositions against the S1 subunit of the SARS-CoV-2 spike protein described herein may be administered by any suitable route of administration, optionally without undue toxicity. A route of administration may refer to any administration pathway known in the art, including but not limited to aerosol, enteral, nasal, ophthalmic, oral, parenteral, rectal, transdermal or vaginal.

In certain embodiments of the present invention, the pharmaceutically acceptable excipient is selected from a group consisting of one or more water-soluble agents, such as lactose, mannitol, calcium sulphate, dextrin, dextrates, dextrose, sucrose, povidone and the like; water-dispersible diluent, such as microcrystalline cellulose, powdered cellulose, starch (corn starch, pregelatinized starch), clay or clay minerals (kaolin, bentonite, attapulgite); glindant, such as calcium carbonate, magnesium stearate, stearic acid and the like; anti-adherent, such as talk, titanium dioxide, red and yellow iron oxide, sodium lauryl sulfate and the like; lubricant, such as silicon dioxide, hydrogenated vegetable oil and the like, preservative, such as butylated hydroxyanisole and the like; suspending agent, such as sodium carboxymethylcellulose, methylcellulose and the like; and so forth.

In certain embodiments of the present invention, the pharmaceutically acceptable adjuvant may be selected from a group consisting of buffering agents, such as citric acid, sodium citrate and the like; preservatives, such as sodium benzoate and the like; anti-cracking agents, such as colloidal silicon dioxide and the like; flavours to mask bitter taste; suspending agents, such as xanthan gum, carrageenan and the like; antifoaming agents, such as simethicone and the like; and colouring agents, such as ferric oxide and the like.

In certain embodiments of the present invention, the aptamer or pharmaceutical composition is administered to the patient in combination with one or more other therapeutic agents. For example, the aptamer or pharmaceutical composition against the S1 subunit of the SARS-CoV-2 spike protein of the invention may be administered in combination with another anti-SARS-CoV2 therapy. For example, the anti-SARS-CoV-2 therapy may be used to treat acute respiratory distress syndrome and/or symptoms associated with acute respiratory distress syndrome.

In certain embodiments, the anti-SARS-CoV-2 therapy is a cell therapy treatment (e.g., treatment with antibodies generated against SARS-CoV-2 therapy obtained and/or purified from another patient). In certain embodiments, the anti-SARS-CoV-2 therapy is a pharmaceutically effective amount of remdesivir, chloroquine, hydroxychloroquine, lopinavir, ritonavir and/or interferon β. In certain embodiments, the anti-SARS-CoV-2 therapy is dexamethasone. In certain embodiments, the anti-SARS-CoV-2 therapy is azithromycin in combination with hydroxychloroquine, favipiravir, meplazumab or tocilizumab. In certain embodiments, the anti-SARS-Cov-2 therapy is tocilizumab or any other suitable anti IL-6 therapy.

In certain embodiments, the invention provides a method of detecting the presence, absence or amount of SARS-CoV-2 in a sample as described herein, wherein if SARS-CoV-2 is detected in the sample obtained from the subject, the method further comprises treating the subject with an anti-SARS-CoV-2 therapy or combination therapy as described herein. For example, the method may further comprise administering a therapeutic aptamer of the invention. The method may further comprise administering a cell therapy. The method may further comprise administering a pharmaceutically effective amount of remdesivir, chloroquine, hydroxychloroquine, lopinavir, ritonavir and/or interferon β.

In the embodiments wherein the aptamer or pharmaceutical composition are used in combination with one or more other therapeutic agents, the aptamer or pharmaceutical composition and the additional therapeutic agents may be administered by the same route or by different routes. For a non-limiting example, the aptamer or pharmaceutical composition may be administered by aerosol while the other therapeutic agents of the combination may be administered by injection.

Kits

The invention also provides a kit for detecting and/or quantifying SARS-CoV-2, wherein the kit comprises one or more aptamers as described herein. Typically, the kit also comprises a detectable molecule as described herein.

In some embodiments, the kit further comprises instructions for use in accordance with any of the methods described herein.

In certain embodiments, the kit further comprises a linker and/or support as described herein.

Typically, the kit comprises further components for the reaction intended by the kit or the method to be carried out, for example components for an intended detection of enrichment, separation and/or isolation procedures. Examples are buffer solutions, substrates for a colour reaction, dyes or enzymatic substrates. In the kit, the aptamer may be provided in a variety of forms, for example pre-immobilised onto a support (e.g., solid support), freeze-dried or in a liquid medium.

The kit of the invention may be used for carrying out any method described herein. It will be appreciated that the parts of the kit may be packaged individually in vials or in combination in containers or multi-container units. Typically, manufacture of the kit follows standard procedures which are known to the person skilled in the art.

In certain embodiments, the kit may be used to capture and/or concentrate the virus as further described herein. For example, the invention may comprise a kit comprising an aptamer as described herein to help improve the sensitivity of any diagnostic tests for COVID-19.

It will be appreciated that features of the present disclosure are susceptible to being combined in various combinations without departing from the scope of the present disclosure as defined by the appended claims.

The present invention further provides the subject matter of the following numbered paragraphs:

1. An aptamer capable of binding to the SARS-CoV-2 spike protein, wherein the aptamer comprises:
(a) a nucleic acid sequence selected from any one of SEQ ID NOs: 1 to 113 or 129 to 144;
(b) a nucleic acid sequence selected from any one of SEQ ID NOs: 145 to 192;
(c) a nucleic acid sequence having at least about 85%, 90%, 95% or more sequence identity with any one of the sequences of (a) or (b); or
(d) a nucleic acid sequence having at least about 15 consecutive nucleotides of any one of the sequences of (a) to (c).

2. The aptamer of paragraph 1, wherein the aptamer comprises:
(a) a nucleic acid sequence selected from any one of SEQ ID NOs: 1, 2, 4 to 6, 8 to 14, 16 to 26, 28 to 46 or 48 to 49;
(b) a nucleic acid sequence having at least about 85%, 90%, 95% or more sequence identity with any one of the sequences of (a); or
(c) a nucleic acid sequence having at least about 15 consecutive nucleotides of any one of the sequence of (a) or (b).

3. The aptamer of paragraph 1, wherein the aptamer is capable of binding to the receptor binding domain (RBD) of the S1 subunit of the SARS-CoV-2 spike protein and comprises:
(a) a nucleic acid sequence selected from any one of SEQ ID NOs 4, 5, 6, 14, 17, 18, 21, 29, 32, 38, 45 or 50;
(b) a nucleic acid sequence selected from any one of SEQ ID NOs 1, 2, 4 to 6, 8 to 12, 14, 16 to 17, 19 to 25, 29, 31 to 32, 35 to 38, 43 to 45 or 48 to 49;
(c) a nucleic acid sequence selected from any one of SEQ ID NOs 4, 5, 6, 14, 17, 21, 29, 32, 38 or 45;
(d) a nucleic acid sequence having at least about 85%, 90%, 95% or more sequence identity with any one of the sequences of (a) to (c); or
(e) a nucleic acid sequence having at least about 15 consecutive nucleotides of any one of the sequence of (a) to (d).

4. The aptamer according to paragraph 1, wherein the aptamer is capable of specifically binding to the RBD of the S1 subunit of the SARS-CoV-2 spike protein and comprises:
(a) a nucleic acid sequence selected from any one of SEQ ID NOs 2, 4, 6, 8, 9, 13, 16, 17, 18, 20, 21, 22, 23, 24, 26, 31, 34, 35, 40 to 46, 48 or 49;
(b) a nucleic acid sequence selected from any one of SEQ ID NOs 4, 6, 17, 18, 21 or 45;
(c) a nucleic acid sequence selected from any one of SEQ ID NOs 2, 4, 6, 8, 9, 16, 17, 20, 21, 22, 23, 24, 31, 35, 43 to 45, 48 or 49;
(d) a nucleic acid sequence selected from any one of SEQ ID NOs 4, 6, 17, 21 or 45;
(e) a nucleic acid sequence selected from any one of SEQ ID NOs 4, 8, 9 or 43;
(f) a nucleic acid sequence selected from any one of SEQ ID NOs 4, 8 or 9;
(g) a nucleic acid sequence having at least about 85%, 90%, 95% or more sequence identity with any one of the sequence of (a) to (f); or
(h) a nucleic acid sequence having at least about 15 consecutive nucleotides of any one of the sequence of (a) to (g).

5. The aptamer according to paragraph 4, wherein the aptamer comprises:
(a) a nucleic acid sequence selected from any one of SEQ ID NOs 4, 8, 9, 16, 21, 23, 31, 43 or 48;
(b) a nucleic acid sequence selected from any one of SEQ ID NOs 9, 21, 23, 24 or 31;
(c) a nucleic acid sequence selected from any one of SEQ ID NOs 9, 21, 23 or 31;
(d) a nucleic acid sequence having at least about 85%, 90%, 95% or more sequence identity with any one of the sequence of (a) to (c); or
(e) a nucleic acid sequence having at least about 15 consecutive nucleotides of any one of the sequence of (a) to (d).

6. The aptamer of paragraph 1, wherein the aptamer is capable of binding to the S1 subunit of the SARS-CoV-2 spike protein and also the homologous protein in SARS and/or MERS, wherein the aptamer comprises:
(a) a nucleic acid sequence selected from any one of SEQ ID NOs 1, 5, 10, 11, 12, 14, 19, 25, 28 to 30, 32, 33, 36 to 39;
(b) a nucleic acid sequence selected from SEQ ID NOs 36 or 38;
(c) a nucleic acid sequence having at least about 85%, 90%, 95% or more sequence identity with any one of the sequences of (a) to (b); or
(d) a nucleic acid sequence having at least about 15 consecutive nucleotides of any one of the sequence of (a) to (c).

7. The aptamer of paragraph 1, wherein the aptamer comprises:
(a) a nucleic acid sequence selected from any one of SEQ ID NOs 129, 130, 131, 132, 133 or 134;
(b) a nucleic acid sequence selected from any one of SEQ ID NOs 135, 136, 137, 138, 139, 140 or 141;
(c) a nucleic acid sequence selected from any one of SEQ ID NOs 142, 143 or 144;
(d) a nucleic acid selected from any one of SEQ ID NOs 132, 134, 140, 142 and 144;
(e) a nucleic acid sequence selected from any one of SEQ ID NOs 134, 140 and 144; or
(f) a nucleic acid sequence having at least about 85%, 90%, 95% or more sequence identity with any one of the sequences of (a) to (e).

8. The aptamer of paragraph 7, wherein the aptamer comprises:
(a) SEQ ID NO 140; or
(b) a nucleic acid sequence having at least about 85%, 90%, 95% or 99% identity with SEQ ID NO 140.

9. The aptamer according to paragraph 1, wherein the aptamer is capable of specifically binding to the S2 subunit of the SARS-CoV-2 spike protein and comprises:
(a) a nucleic acid sequence selected from any one of SEQ ID NOs: 145 to 192;
(b) a nucleic acid sequence selected from any one of SEQ ID NOs 146, 150 or 171;
(c) a nucleic acid sequence selected from any one of SEQ ID NOs: 177, 183 or 191;
(d) a nucleic acid sequence selected from any one of SEQ ID NOs 179, 188 or 190;
(e) a nucleic acid sequence having at least about 85%, 90%, 95% or more sequence identity with any one of the sequence of (a) to (d); or
(f) a nucleic acid sequence having at least about 15 consecutive nucleotides of any one of the sequence of (a) to (e).

10. The aptamer of paragraph 9, wherein the aptamer comprises:
(a) SEQ ID NO 190; or
(b) a nucleic acid sequence having at least about 85%, 90%, 95% or 99% identity with SEQ ID NO 190.

11. A pair of aptamers, comprising a first aptamer as defined in any one of paragraphs 1 to 8 capable of binding to the S1 subunit of the SARS-CoV-2 spike protein and a second aptamer as defined in any one of paragraphs 1 to 8 capable of binding to a different or non-competing region of the S1 subunit or a second aptamer as defined in any one of paragraphs 9 to 10 capable of binding to the S2 subunit of the SARS-CoV-2 spike protein.

12. The pair of aptamers of paragraph 11, wherein the aptamers comprise:
(a) a first aptamer comprising a nucleic sequence selected from any one of SEQ ID NOs 4, 8, 9, 134, 144 or 140 or a sequence having at least about 85%, 90%, 95% or more sequence identity or more with any one of SEQ ID NOs 4, 8, 9, 134, 144 or 140; and
(b) (i) a second aptamer comprising a nucleic sequence selected from any one of SEQ ID NOs 4, 8, 9, 134, 144 or 140 or a sequence having at least about 85%, 90%, 95% or more sequence identity or more with any one of SEQ ID NOs 4, 8, 9, 134, 144 or 140, wherein the second aptamer is different than the first aptamer; or
   (ii) a second aptamer comprising a nucleic sequence selected from any one of SEQ ID NOs 146, 150, 171, 177, 179, 183, 188, 190 or 191 or a sequence having at least about 85%, 90%, 95% or more sequence identity with any one of SEQ ID NOs 146, 150, 171, 177, 179, 183, 188, 190 or 191.

13. The pair of aptamers of paragraph 12, wherein the aptamers comprise:
(a) a first and second aptamer comprising nucleic acid sequences selected from SEQ ID NO: 4 and 146, 4 and 150, 4 and 171, 4 and 177, 4 and 179, 4 and 183, 4 and 188, 4 and 190 or 4 and 191 respectively;
(b) a first and second aptamer comprising nucleic acid sequences selected from SEQ ID NO: 8 and 146, 8 and 150, 8 and 171, 8 and 177, 8 and 179, 8 and 183, 8 and 188, 8 and 190 or 8 and 191 respectively;
(c) a first and second aptamer comprising nucleic acid sequences selected from SEQ ID NO: 9 and 146, 9 and 150, 9 and 171, 9 and 177, 9 and 179, 9 and 183, 9 and 188, 9 and 190 or 9 and 191 respectively;
(d) a first and second aptamer comprising nucleic acid sequences selected from SEQ ID NO: 43 and 146, 43 and 150, 43 and 171, 43 and 177, 43 and 179, 43 and 183, 43 and 188, 43 and 190 or 43 and 191 respectively;
(e) a first and second aptamer comprising nucleic acid sequences selected from SEQ ID NO: 134 and 146, 134 and 150, 134 and 171, 134 and 177, 134 and 179, 134 and 183, 134 and 188, 134 and 190 or 134 and 191 respectively;
(f) a first and second aptamer comprising nucleic acid sequences selected from SEQ ID NO: 144 and 146, 144 and 150, 144 and 171, 144 and 177, 144 and 179, 144 and 183, 144 and 188, 144 and 190 or 144 and 191 respectively;
(g) a first and second aptamer comprising nucleic acid sequences selected from SEQ ID NO: 140 and 146, 140 and 150, 140 and 171, 140 and 177, 140 and 179, 140 and 183, 140 and 188, 140 and 190 or 140 and 191 respectively;
(h) a first and/or second aptamer comprising nucleic acid sequences having at least about 85%, 90%, 95% or more sequence identity or more with any of the aptamers of (a) to (g); or
(i) a first and/or second aptamer comprising nucleic acid sequences having at least about 15 or more consecutive nucleotides of any one of the aptamers of (a) to (g).

14. The pair of aptamers of paragraph 13, wherein the aptamer comprises:
(a) a first aptamer comprising a nucleic sequence selected from SEQ ID NO 140 or a sequence having at least about 85%, 90%, 95%, 99% or more sequence identity with SEQ ID NO 140; and
(b) a second aptamer comprising a nucleic sequence selected from SEQ ID NO 190 or a sequence having at least 85%, 90%, 95%, 99% or more sequence identity with SEQ ID NO 190.

15. The aptamer(s) of any one of paragraphs 1 to 14, wherein the aptamer is a DNA aptamer.

16. An aptamer that competes for binding to the SARS-CoV-2 spike protein with the aptamer(s) of any one of paragraphs 1 to 15.

17. The aptamer(s) of any one of paragraphs 1 to 16, wherein the one or more aptamer(s) comprise a detectable label, optionally wherein the detectable label is selected from a fluorophore, a nanoparticle, a quantum dot, an enzyme, a radioactive isotope, a pre-defined sequence portion, a biotin, a desthiobiotin, a thiol group, an amine group, an azide, an aminoallyl group, a digoxigenin, an antibody, a catalyst, a colloidal metallic particle, a colloidal non-metallic particle, an organic polymer, a latex particle, a nanofiber, a nanotube, a dendrimer, a protein, and a liposome.

18. A complex comprising one or more aptamers of any preceding paragraph and a detectable molecule.

19. A biosensor, assay plate or test strip comprising one or more aptamers as described in any one of paragraphs 1 to 17.

20. Apparatus for detecting the presence, absence or level of SARS-CoV-2 in a sample, the apparatus comprising one or more aptamers as described in any one of paragraphs 1 to 17 optionally wherein the apparatus further comprises a support.

21. A lateral flow device comprising one or more aptamers as described in any one of paragraphs 1 to 17.

22. The lateral flow device according to paragraph 21, wherein the device comprises:
(a) a first aptamer comprising a nucleic sequence selected from SEQ ID NO 140 or a sequence having at least about 85%, 90%, 95%, 99% or more sequence identity with SEQ ID NO 140; and
(b) a second aptamer comprising a nucleic sequence selected from SEQ ID NO 190 or a sequence having at least 85%, 90%, 95%, 99% or more sequence identity with SEQ ID NO or 190.

23. The device according to paragraph 22, wherein the first or second aptamer is bound to a solid support, optionally wherein the solid support is a test strip.

24. The device according to paragraph 23, wherein:
(i) if the first aptamer is bound to the solid support, the second aptamer is conjugated to a detectable label; or
(ii) if the second aptamer is bound to the solid support, the first aptamer is conjugated to a detectable label.

25. The device according to paragraph 24, wherein the detectable label is a gold or latex nanoparticle.

26. A functionalised electrode or biosensor surface comprising one or more aptamers as described in any one of paragraphs 1 to 17.

27. The functionalised electrode or biosensor according to paragraph 26, wherein the functionalised electrode or biosensor comprises:
(a) an aptamer comprising a nucleic sequence selected from SEQ ID NO 10, 20, 24, 44 or 134; or
(b) a sequence having at least about 85%, 90%, 95%, 99% or more sequence identity with SEQ ID NO 10, 20, 24, 44 or 134.

28. The functionalised electrode or biosensor according to paragraph 27, wherein the functionalised electrode or biosensor comprises:
(a) an aptamer comprising a nucleic sequence selected from SEQ ID 134; or
(b) a sequence having at least about 85%, 90%, 95%, 99% or more sequence identity with SEQ ID NO 134.

29. The functionalised electrode or biosensor according to paragraph 27 or 28, wherein the aptamer is immobilised onto the surface of the electrode, optionally wherein the surface of the electrode is gold.

30. Use of one or more aptamers as described in any one of paragraphs 1 to 17, a complex as described in paragraph 18, a biosensor or test strip as described in paragraph 19, apparatus as described in paragraph 20, lateral flow device as described in any one of paragraphs 21 to 25 or functionalised electrode or biosensor as described in any one of paragraphs 26 to 29 for detecting, enriching, separating and/or isolating SARS-CoV-2.

31. The use according to paragraph 30, wherein SARS-CoV-2 is enriched in a sample prior to detecting the presence, absence or amount of the SARS-CoV-2 in the sample.

32. A method of detecting the presence, absence or amount of SARS-CoV-2 in a sample, the method comprising:
(i) interacting the sample with an aptamer of any one of paragraphs 1 to 17; and
(ii) detecting the presence, absence or amount of SARS-CoV-2.

33. The method of paragraph 32, wherein the sample is obtained from a subject having or suspected of having a COVID-19 infection.

34. The method of paragraph 33, wherein the sample is saliva, blood, or a nasal swab.

35. The method of any one of paragraphs 32 to 34, wherein an infecting viral load of SARS-CoV-2 is detected.

36. A kit for detecting, quantifying and/or enriching SARS-CoV-2, the kit comprising one or more aptamers as described in any one of paragraphs 1 to 17.

37. The kit of paragraph 36, wherein the kit comprises a linker, support and/or detectable molecule.

### EXAMPLES

In the following, the invention will be explained in more detail by means of non-limiting examples of specific embodiments. In the example experiments, standard reagents and buffers free from contamination are used.

### Example 1—Aptamer Selection

Aptamer Selection Target Protein Preparation

Recombinant, His tagged S1 subunit of the SARS-CoV-2/2019-nCoV Spike Protein (SEQ ID NO: 118) was supplied by SinoBiological (catalogue number 40591-V08B1). The His tagged RBD of the S1 subunit (SEQ ID NO: 120) was also supplied by SinoBiological (catalogue number 40592-VO8H).

The protein was characterised by UV spectroscopy and SDS-PAGE analysis for quality control purposes. The Spike proteins were immobilised onto separate batches of Dynabeads™ His-Tag Isolation and Pulldown magnetic beads (Thermo Fisher Scientific, UK), according to manufacturer's protocols. The protein loading density was determined spectrophotometrically. This process was repeated with S1 subunit of the SARS-CoV Spike Protein and the S1 subunit of the MERS-CoV Spike Protein, also provided by SinoBiological.

Aptamer Library and Oligonucleotides

The aptamer selection process was carried out, starting from a synthetic ssDNA oligonucleotide sequences of an aptamer library (manufactured by IDT, Belgium). The nucleotide sequences of the aptamer library have the following structure (in a 5′ to 3′ direction):
P1-R-P2,

wherein P1 is a first primer region, R is a randomized region (40 nucleotides in length in this case) and P2 is a further primer region wherein R or a portion thereof are involved in target molecule binding.

The following modified primers were used in the amplification of the oligomers by means of PCR: fluorescein (FAM)-labelled forward primer (P1) with the sequence: 5′-/56FAM/CCAGTGTAGACTACTCAATG-3′ (SEQ ID NO: 114) and PO₄-modified reverse primer (P2) with the sequence: 5′-/5Phos/GGTTGACCTGTGGATAGTAC-3′ (SEQ ID NO: 116).

Establishing Parameters for Aptamer Selection

A preliminary study was carried out to identify the most appropriate selection buffer and required target excess for the selection process against the S1 subunit of the SARS-CoV-2 spike protein and separately against the RBD. The naïve aptamer library was diluted into each of three aptamer selection buffers and aliquoted across a 96 well plate, such that each well contains 1.65 picomoles of buffered aptamer library. A concentration gradient of the S1 or RBD immobilised Dynabeads was prepared and aliquoted across separate 96 well plates, to give a seven-point target gradient consisting of different ‘fold-excess’ of target (and a row of ‘target-negative’ controls). An equivalent gradient of target-negative beads (blank Dynabeads with nothing immobilised) was also prepared. The binding assay was carried out on Biomek NX liquid handling robots (Beckman Coulter). In this binding assay, the buffered aptamer library is added to the bead gradient, these are mixed and incubated for one hour at room temperature, to allow aptamers to bind to the immobilised target or blank beads. After incubation, the beads are separated on a 96 well magnetic plate rack, and unbound aptamers are removed. The beads are washed with the respective buffer, to remove any weak binding aptamers. The bound aptamers are recovered from the beads by resuspending the beads in PCR mix and thermal-cycled (according to standard manufacturer protocols). This results in the elution and amplification of the bound aptamers. The amount of aptamers recovered from each target concentration is then quantified by qPCR. ‘Recovery data’ is compared to determine the amount of aptamer bound to the target immobilised and blank beads, in each of the selection buffers. The ‘optimum’ selection buffer is chosen based on the ratio of aptamer biding to target loaded vs blank beads. The amount of target to be used in selection is also chosen from this assay, based on the amount of aptamer recovered. For the aptamer selection against both S1 subunit of the SARS-CoV-2 Spike protein and RBD, the best performing buffer is 50 mM MES pH6.2, 5 mM MgCl₂, 1 mM CaCl₂), 20 mM NaCl, 4.5 mM KCl, 20 mM Na₂SO₄, 0.01% (v/v) Tween-20, 0.01% (w/v) BSA. In both cases, a 50-fold excess of target was chosen for the first round of selection.

In Vitro Selection of an Aptamer Population Against the S1 Subunit of the SARS-CoV-2 Spike Protein

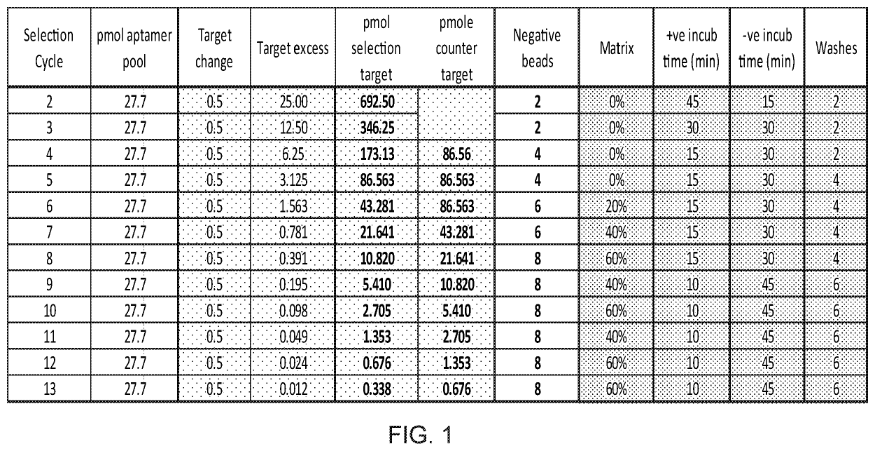

The selection process consisted of iterative selection rounds with increasingly stringent selection conditions (defined in FIG. 1). In Cycle 1, 166 pmol of naïve aptamer library is incubated with the target immobilised beads, using the binding conditions established in the preliminary binding study. The beads are washed to remove loosely bound aptamers, then remaining aptamers are eluted in PCR mix and amplified as carried out in the preliminary binding study. The recovered amplified aptamer library is purified using AxyPrep Mag PCR Clean-up Kit (Axygen Biosciences, USA), digested with Lambda exonuclease (EURx, Poland) at 37° C. according to manufacturers' protocol. The nascent ssDNA is purified using AxyPrep Mag PCR Clean-up kit to produce a purified and enriched single stranded DNA library for the subsequent aptamer selection cycle. In Cycle 2 (and all subsequent rounds), the same process is followed but aptamer-target incubations are carried out with increasingly stringent conditions. The details of this ‘stringency map’ (target ratios, incubation times, washes etc) are defined in FIG. 1. At Cycle 6, counter selection against the S1 subunit of the MERS spike protein was introduced. In this selection cycle, the aptamer library was incubated with beads derivatised with S1 subunit of the MERS spike protein. After incubation, the MERS beads were separated and aptamers which do not bind to the MERS protein were recovered and incubated with the SARS-CoV-2 S1 target loaded beads. The selection stringency map shown in FIG. 1 gives details of the counter selection steps. As the aptamer selection progresses the target excess and positive incubation time decreases, and the negative/counter excess and negative incubation time increases. The number of washes also increases, to increase selection stringency.

In Vitro Selection of an Aptamer Population Against the S2 Subunit of the SARS-CoV-2 Spike Protein

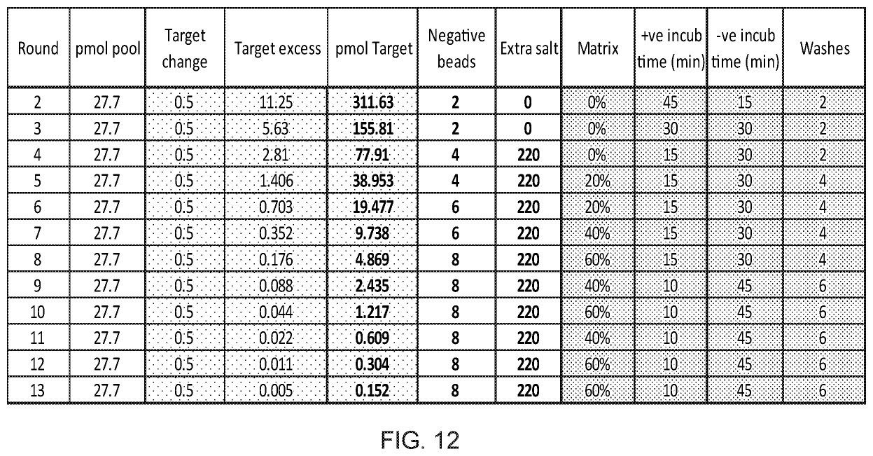

Aptamer selection against the S2 subunit of the SARS-CoV-2 spike protein was carried out as for the S1 subunit of the SARS-CoV-2 spike protein (described above) using selection conditions as described in FIG. 12.

### Example 2—Characterisation of the Aptamer Population

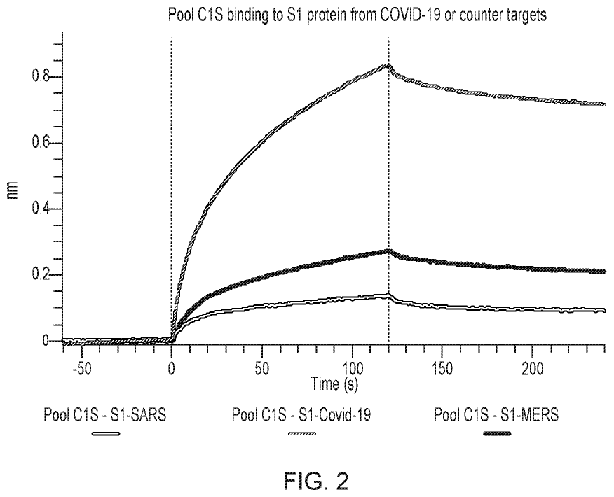

After aptamer selection, the refined aptamer populations were assessed for the ability to bind to the respective SARS-CoV-2 protein using a Biolayer interferometry assay. The experiments described here were conducted using an Octet RED384 instrument (ForteBio, Pall Life Sciences, USA) based on manufacturers defined protocols. The aptamer population was prepared using a biotinylated primer (SEQ ID NO: 114), in the PCR reaction. Biotinylated ssDNA was then immobilised onto the surface of streptavidin coated biosensor probes (Streptavidin-SA Dip & Read Biosensors, ForteBio, Pall Life Sciences, USA) following manufacturer protocols. The aptamer populations were prepared at 50 nM in 1× ‘high salt’ aptamer selection buffer (50 mM MES pH6.2, 5 mM MgCl₂, 1 mM CaCl₂), 220 mM NaCl, 4.5 mM KCl, 20 mM Na₂SO₄, 0.01% (v/v) Tween-20, 0.01% (w/v) BSA). Target protein stocks were also prepared in the 1× ‘high salt’ aptamer selection buffer. All buffer/blank/baseline interactions were carried out in 1× ‘high salt’ aptamer selection buffer with no added spike protein etc. The interaction between the immobilised aptamer population and S1 subunit of the spike protein from either SARS-COV-2, SARS or MERS was monitored (FIG. 2). All data was reference corrected using a blank sensor probe (no immobilised aptamer) to allow correction of buffer effects.

Cloning

After the target binding was confirmed for the selected aptamer population; the recovered aptamer library was amplified by PCR, using unmodified forward and reverse primers (SEQ ID NO: 114 and SEQ ID NO: 116). The purified dsDNA was cloned into the pJET1.2/blunt cloning vector, following manufacturers protocol (CloneJET PCR cloning kit, ThermoFisher Scientic, UK) and used to transform a sequencing strain of E. coli (NEB 5-alpha E. coli C2987H cells) 96 positive transformants/colonies were ‘picked’ and analysed by ‘colony PCR’, using plasmid-specific primer (pJET forward primer and pJET reverse primer (CloneJET PCR cloning kit, Thermo Fisher Scientic, UK). In parallel, aptamer DNA was produced from the same transformants/clones by ‘aptamer PCR’ using aptamer specific FAM-labelled forward primer and PO4-modified reverse primer.

Identification of Individual Aptamers

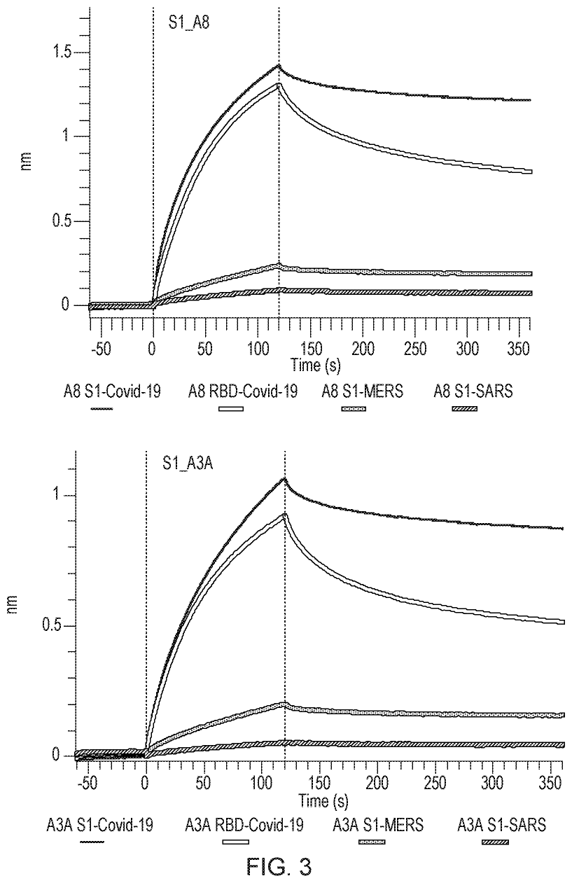

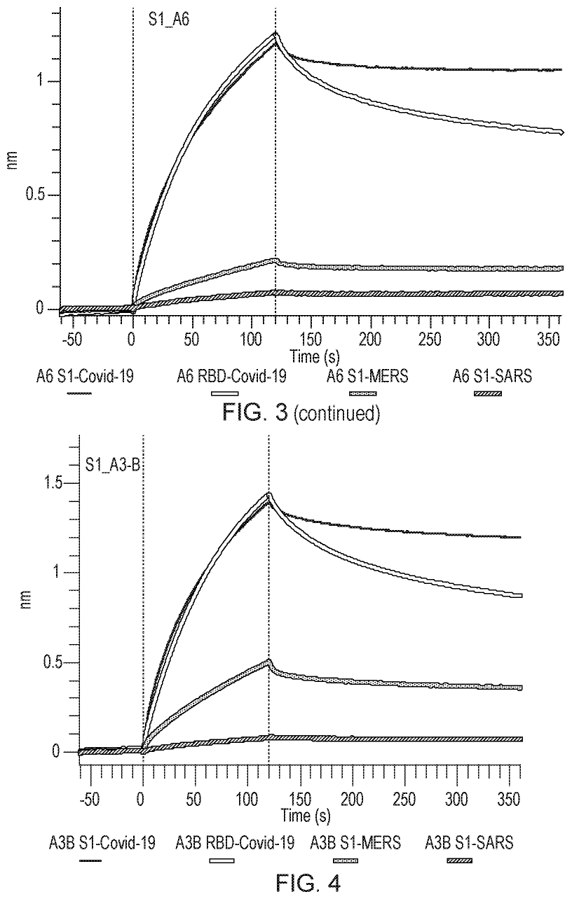

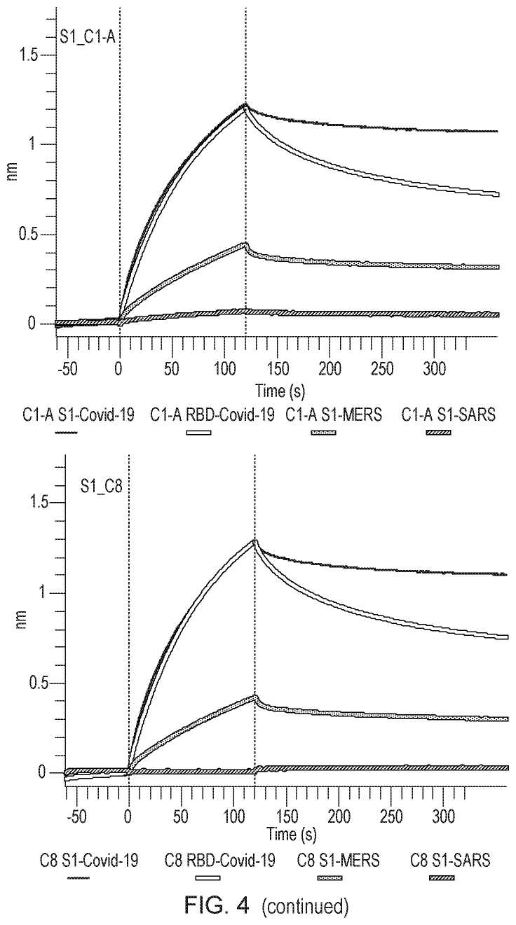

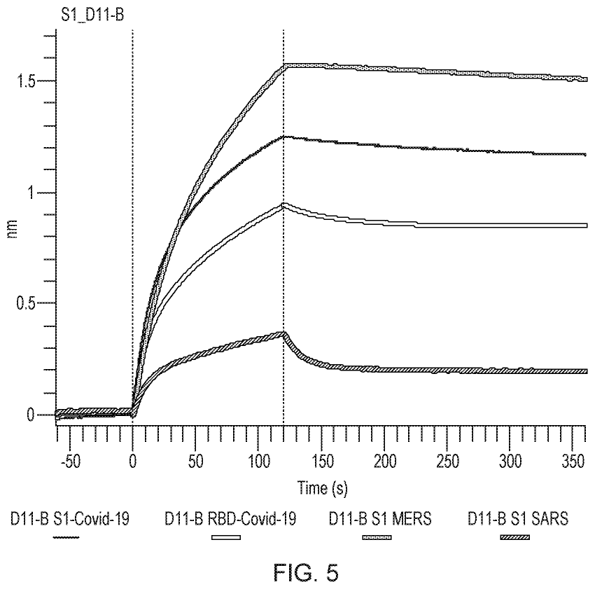

Single stranded DNA was prepared for each of the individual aptamers derived from the aptamer pool according to cloning protocol above. Each clone was then analysed for binding to the S1 subunit of the spike protein from SARS-CoV-2, SARS, MERS or the RBD protein from SARS-CoV-2; using the BLI assay described above. The aptamers broadly fall into a number of different categories; for example, those which bind to S1 subunit of the spike protein from SARS-CoV-2 but not from SARS or MERS (FIG. 3); those which bind to S1 subunit of the spike protein from SARS-CoV-2 and weakly cross react with MERS (FIG. 4); and those which bind to S1 subunit of the spike protein from SARS-CoV-2 and cross react with SARS and MERS (FIG. 5). The DNA sequence of all screened clones was determined by Sanger Sequencing of the ‘colony PCR’ product (described above) by DNA Sequencing and Services, (University of Dundee, UK). The obtained sequence data was analysed and aligned by using the web-based tool ClustalW provided by the EBI web server (http://www.ebi.ac.uk/Tools/msa/clustalw2/).

As described above, FIGS. 3 to 5 include representative examples of aptamers that fall within the different categories. An overview of the various properties of the selected aptamers including those that show unexpectedly improved binding and/or affinity as compared to other aptamers identified during the selection process is shown in Table 1 below.

| | | SARS-CoV-2 | | | |
|---|---|---|---|---|---|
| SEQ ID NO: | Clone Name | S1 | RBD | MERS | SARS |
| 1 | S1_A1-A | ++ | ++ | + | − |
| 2 | S1_A1-B | ++ | ++ | − | − |
| 4 | S1_A3-A | ++ | ++ | − | − |
| 5 | S1_A3-B | ++ | ++ | + | − |
| 6 | S1_A4 | ++ | ++ | − | − |
| 8 | S1_A6 | ++ | ++ | − | − |
| 9 | S1_A8 | ++ | ++ | − | − |
| 10 | S1_A9 | ++ | ++ | + | − |
| 11 | S1_A12 | ++ | ++ | + | − |
| 12 | S1_B1 | ++ | ++ | + | − |
| 13 | S1_B3 | + | + | − | − |
| 14 | S1_B4 | ++ | ++ | + | − |
| 16 | S1_B9 | ++ | ++ | − | − |
| 17 | S1_B11-A | ++ | ++ | − | − |
| 18 | S1_B12-B | − | + | − | − |
| 19 | S1_C1-A | ++ | ++ | + | − |
| 20 | S1_C1-B | ++ | ++ | − | − |
| 21 | S1_C2 | ++ | ++ | − | − |
| 22 | S1_C3-A | − | ++ | − | − |
| 23 | S1_C3-B | ++ | ++ | − | − |
| 24 | S1_C4 | ++ | ++ | − | − |
| 25 | S1_C8 | ++ | ++ | + | − |
| 26 | S1_C9 | + | + | − | − |
| 28 | S1_C11 | + | + | + | − |
| 29 | S1_C12 | ++ | ++ | + | − |
| 30 | S1_D1 | + | + | + | − |
| 31 | S1_D2 | ++ | ++ | − | − |
| 32 | S1_D4 | ++ | ++ | + | − |
| 33 | S1_D6 | − | + | + | − |
| 34 | S1_D8 | + | + | − | − |
| 35 | S1_D10-A | ++ | ++ | − | − |
| 36 | S1_D11-B | ++ | ++ | ++ | + |
| 37 | S1_E2 | ++ | ++ | + | − |
| 38 | S1_E3 | ++ | ++ | ++ | − |
| 39 | S1_E5 | − | + | + | − |
| 40 | S1_E6 | − | + | − | − |
| 41 | S1_E9 | + | + | − | − |
| 42 | S1_E10 | + | + | − | − |
| 43 | S1_F2 | ++ | ++ | − | − |
| 44 | S1_F3-A | ++ | ++ | − | − |
| 45 | S1_F3-B | ++ | ++ | − | − |
| 46 | S1_F5 | + | + | − | − |
| 48 | S1_F11 | ++ | ++ | − | − |
| 49 | S1_F12 | ++ | ++ | − | − |

Key to Table 1:

| | |
|---|---|
| ++ | Strongest binding |
| + | Moderate binding |
| − | Low or no significant binding |

The categorisation of the S1 targeting aptamers was carried out using aptamers produced by solid phase synthesis (incorporating a biotin group at the 5′ end of the aptamer sequence). This resulted in clearer binding data and enabled clear characterisation and categorisation of the isolated individual aptamers. Consequently, the binding of aptamers—the sequences of which were identified through aptamer selection against S1 subunit of the spike protein—was analysed for various targets (including SARS-CoV-2 (RBD and S1 subunit), MERS and SARS) and the binding categorised as either strongest, moderate, or low or no significant binding. Aptamers were split into these categories based on the signal response in the BLI assays. Aptamers with target interaction response >greater than 1 nm was classified as ‘Strong’; ˜0.5 nm was classified as ‘moderate’; less than ˜0.3 nm was classified as ‘Low or no significant binding’.

Determination of Aptamer Binding Affinity to the S1 Subunit of the SARS-CoV-2 Spike Protein

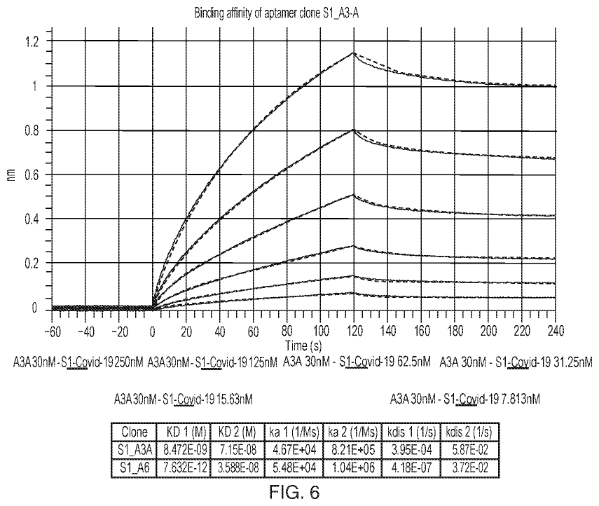

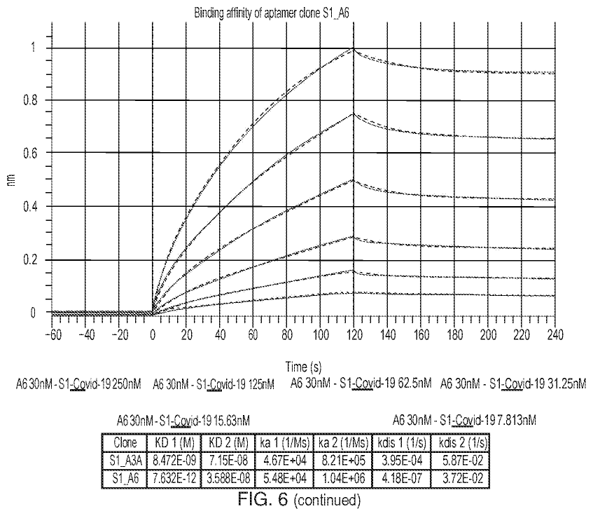

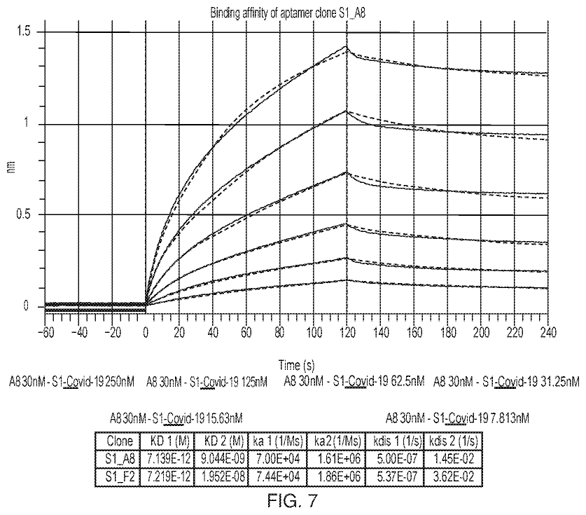

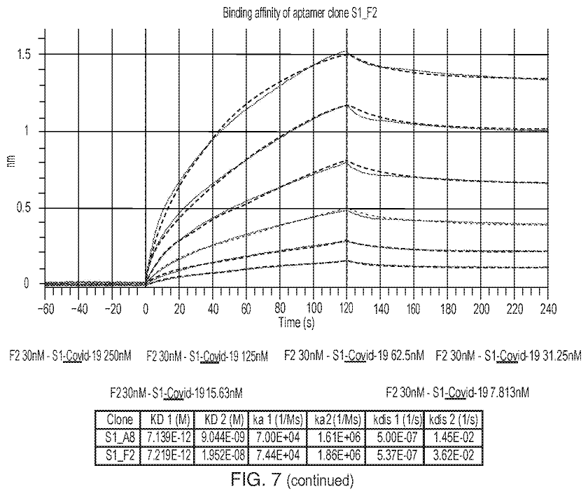

The affinity of four chosen aptamers (aptamer S1_A3-A (SEQ ID NO: 4); aptamer S1_A6 (SEQ ID NO: 8); aptamer S1_A8 (SEQ ID NO: 9); aptamer S1_F2 (SEQ ID NO: 43)) was determined using the BLI assay according to the protocol described above. The biotinylated aptamers were immobilised onto streptavidin biosensor probes (as described above) and then interaction monitored with a concentration series of SARS-CoV-2 Spike protein S1 (250 nM, 125 nM, 62.5 nM, 31.25 nM, 15.63 nM, 7.81 nM, 0 nM diluted in 1× ‘high salt’ aptamer selection buffer). K_{D} values were calculated using the ForteBio software (ForteBio Data Analysis 8.0,), using a 2:1 binding model (FIGS. 6 & 7).

Identification of Minimal Functional Aptamer Fragment

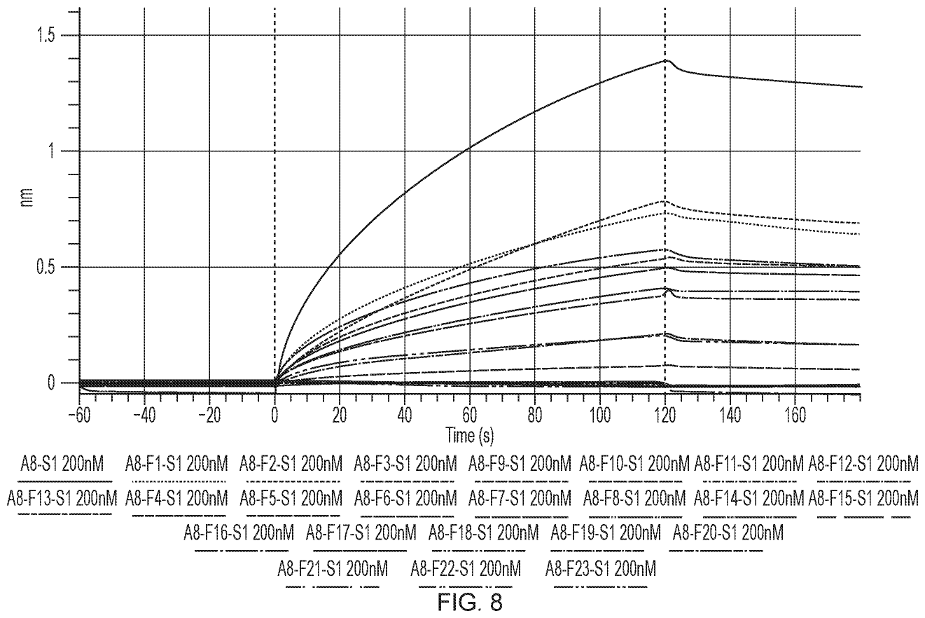

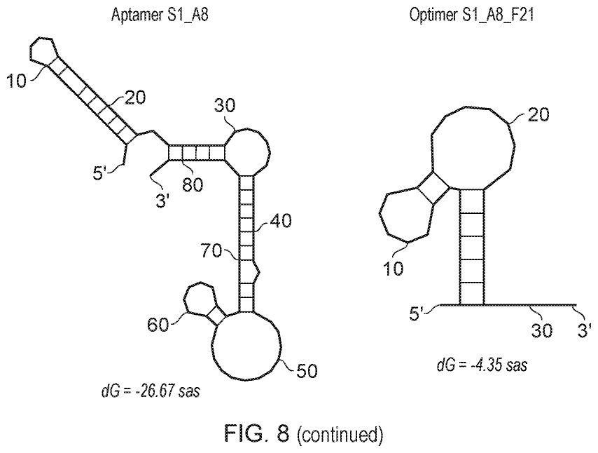

A minimal functional aptamer fragment (Optimer™) was identified for several of the isolated and characterised aptamers. Briefly, a panel of fragments representing different regions of the full-length aptamer, were produced by solid phase synthesis (again incorporating a 5′ biotin group). Each of these individual fragments was immobilised onto a separate streptavidin coated BLI sensor probe, and the interaction with the buffered S1 subunit of the SARS-CoV-2 spike protein was monitored using the BLI assay (according to the protocol described above). The BLI screen shows which fragments retain their binding affinity and which fragments have lost their binding function. These binding and non-binding fragments are mapped onto the full-length aptamer sequence to identify the minimal functional fragment (Optimer™). Example data for aptamer S1_A8 (SEQ ID NO: 9) and S1_A8_F21 (SEQ ID NO: 140) is given in FIG. 8 and clearly demonstrates the identification of a minimal aptamer fragment which retains the ability to bind to the SARS-CoV-2 protein.

### Example 3—Optimers Isolated Against the S1 Subunit of the SARS-CoV-2 Spike Protein, Bind to Full Spike Protein

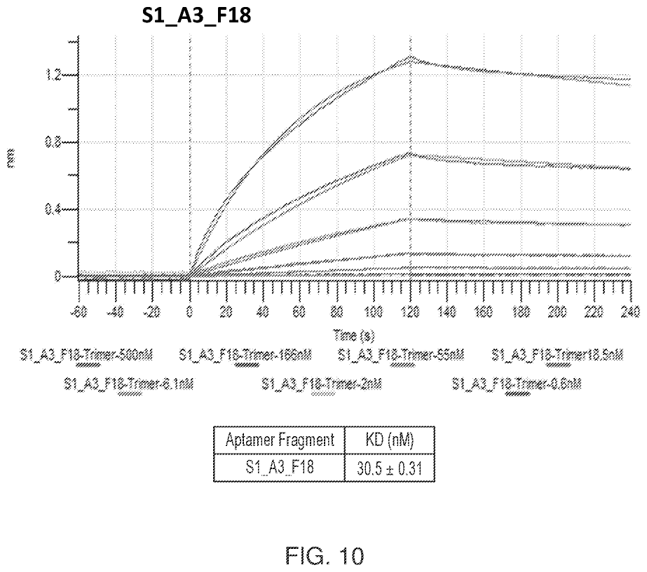

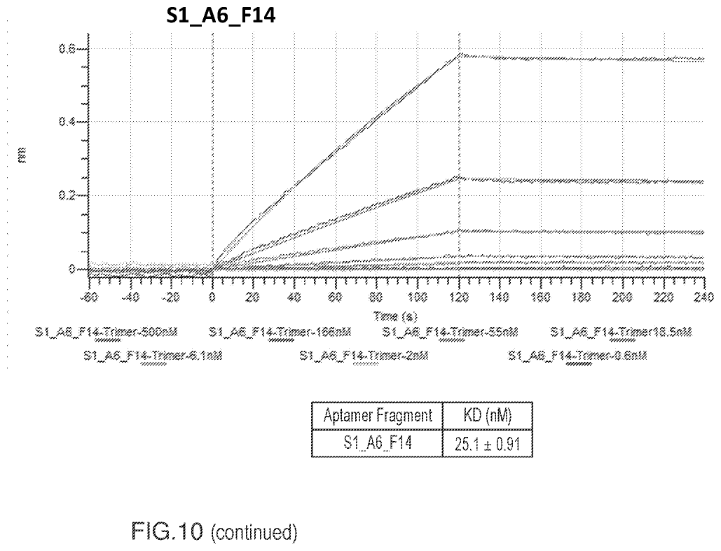

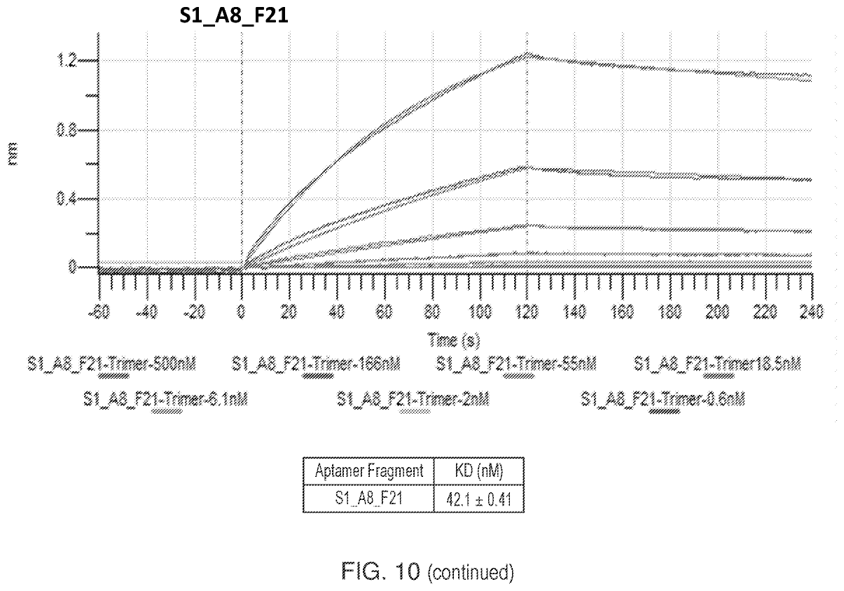

The aptamers described herein have been isolated using recombinant proteins which represent portions of the full viral surface spike protein (SEQ ID NO: 117). For diagnostic and therapeutic applications of the aptamers, it is desirable that the aptamers and/or minimal functional fragment Optimers are capable of binding to the spike protein in the full trimeric form. For example, any aptamers or Optimers which bind to regions of the monomer which are not accessible in the trimeric spike may not be capable of binding to the virus and be less likely to perform well as a diagnostic or therapeutic. Several of the isolated Optimers isolated against the S1 subunit of the SARS-CoV-3 spike protein, were assessed for binding to the full trimeric spike protein using the BLI assay described above. Briefly, exemplar Optimers were immobilised onto BLI sensor probes and assessed for the ability to bind to the full SARS-CoV-2 spike protein trimer at a range of concentrations (500 nM, 166 nM, 55 nM, 18.5 nM, 6.1 nM, 2 nM and 0.6 nM in 1× ‘high salt’ aptamer selection buffer). Data shown in FIG. 10 demonstrates a clear concentration dependant interaction between the immobilised Optimer and spike protein; indicating that each of the aptamers have high affinity (˜20-40 nM) for the full spike protein trimer. Importantly, each of the aptamers (SEQ ID NO: 134, 140 and 144) has a rapid association rate (0-120 sec) and a slow dissociation rate (120-240 sec); which may be important for many diagnostic applications.

### Example 4—Aptamers and Optimers Bind to S1 Subunit of the SARS-CoV-2 Spike Protein in Buffered Saliva

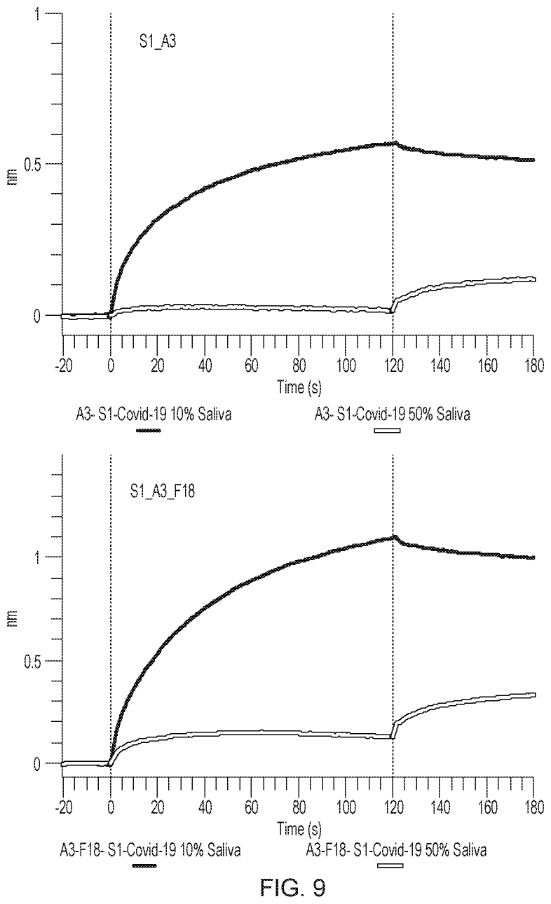

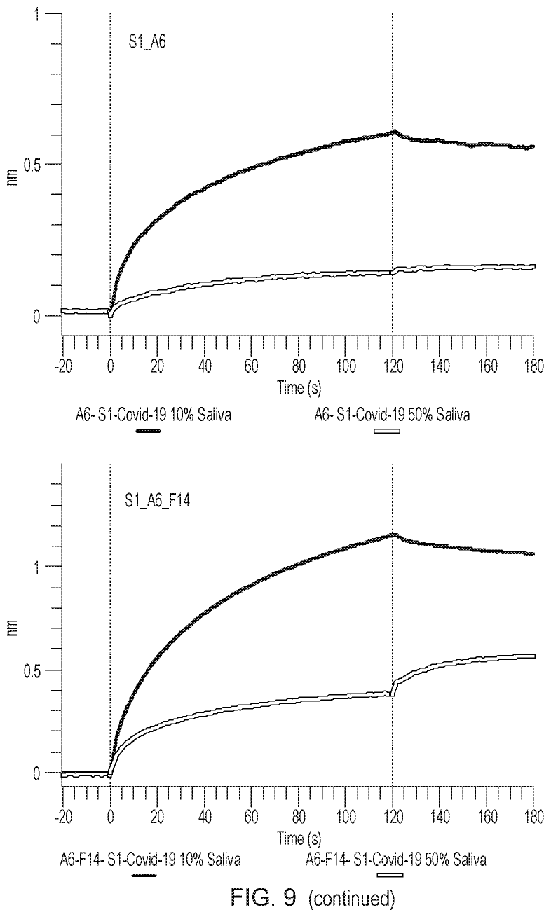

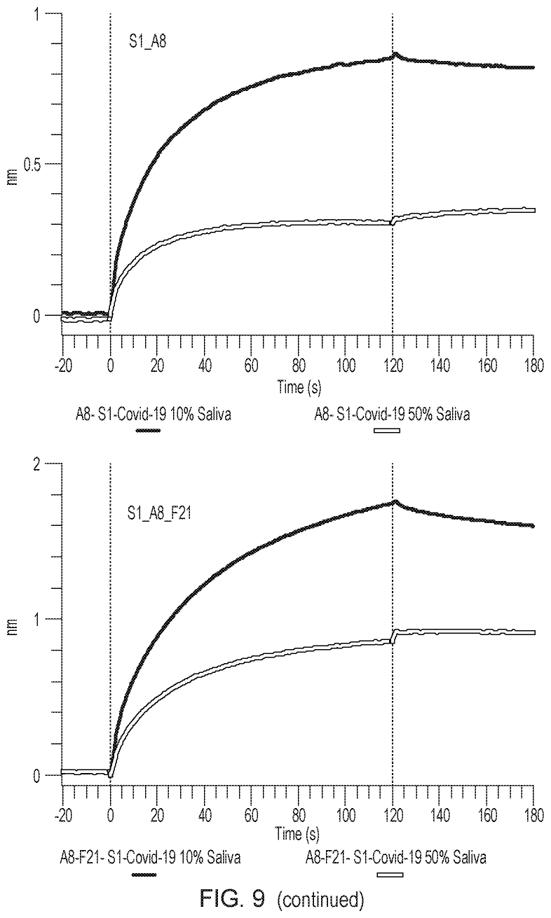

In order to use the selected aptamers in simple point-of-care diagnostic; it is desirable that the aptamers and Optimers are capable of recognising and binding to their target in a readily accessible sample matrix, with minimal processing. To demonstrate this proof-of-concept, the aptamers and respective Optimers, were immobilised onto BLI sensor probes and assessed for the ability to bind to the S1 subunit of the SARS-CoV-2 spike protein when spiked into samples of buffered saliva. Briefly; saliva samples were obtained from healthy volunteers, combined in equal volumes and then buffered with 1× ‘high salt’ aptamer selection buffer (to a final concentration of 1×). The buffered saliva was prepared at both 10% and 50% (v/v) saliva, spiked with the S1 subunit of the SARS-CoV-2 spike protein (at 0.5 μM). Spiked saliva samples were incubated with the immobilised aptamers or Optimers and interactions monitored using the BLI binding assay described above (FIG. 9). BLI data shows a clear interaction between the immobilised aptamers and Optimers, and the S1 subunit of the SARS-CoV-2 spike protein in saliva. This demonstrates the potential of these aptamers and Optimers to recognise and bind the virus in patient saliva samples; without the need for expensive or laborious sample processing steps.

An overview of the various properties of the selected aptamer fragments including those that show strongest binding of S1 and/or binding to spike S1 in saliva (described above) is shown in Table 3 below:

| SEQ ID NO: | Optimer Name | S1 | Saliva binding |
|---|---|---|---|
| 129 | S1_A3_F9 | ++ | |
| 130 | S1_A3_F10 | ++ | |
| 131 | S1_A3_F11 | ++ | |
| 132 | S1_A3_F12 | ++ | ++ |
| 133 | S1_A3_F17 | ++ | |
| 134 | S1_A3_F18 | ++ | ++ |
| 135 | S1_A8_F2 | ++ | |
| 136 | S1_A8_F12 | ++ | |
| 137 | S1_A8_F13 | ++ | |
| 138 | S1_A8_F14 | ++ | |
| 139 | S1_A8_F20 | ++ | |
| 140 | S1_A8_F21 | ++ | ++ |
| 141 | S1_A8_F22 | ++ | |
| 142 | S1_A6_F12 | ++ | ++ |
| 143 | S1_A6_F13 | ++ | |
| 144 | S1_A6_F14 | ++ | ++ |

### Example 5—S1 Binding Aptamers Specifically Bind to the S1 Subunit of the SARS-CoV-2 Spike Protein (ELONA Assay)

Before developing a sandwich assay; it is usual to assess the performance of any affinity ligand (aptamer, antibody or other) in a simpler assay format. In this case, an indirect ELONA was first carried out to assess the binding and specificity of the SARS-CoV-2 S1 binding aptamers. S1 subunits from the spike proteins from SARS-Cov-2, SARS and MERS were each immobilised onto separate wells of a standard protein binding ELISA plate (MaxiSorp™ plate, Nunc®). A row of wells was left blank (no protein) to allow background subtraction of non-specific aptamer binding. After overnight incubation, the unbound protein was removed by washing the wells with 1× ‘high salt’ aptamer selection buffer. After blocking the remaining sites on the plate (with 1% BSA in 1×PBS), the protein immobilised wells were incubated with either full-length S1 binding Aptamer A3-A, Aptamer A6 or Aptamer A8 (SEQ ID NO: 4, 8, 9 respectively); or minimal functional fragment A3_F18 (SEQ ID NO: 134). Each of the aptamers was synthesised with a biotin group on the 5′ end to allow subsequent capture of the streptavidin-HRP conjugate protein. After aptamer incubation for 90 mins at room temperature; unbound aptamer was removed by washing the wells with 1× ‘high salt’ aptamer selection buffer. Wells were then incubated with strep-HRP (0.2 μg/ml in 1×PBS) to capture the enzyme on any protein bound aptamers. After washing wells to remove unbound Strep-HRP; each of the wells was incubated with the substrate, 3,3′, 5,5′-tetramethylbenzidine (TMB) which yields a blue colour when cleaved by HRP. Termination of the enzymatic activity (with 1M HCl) yields a yellow product which can be quantified by measuring the absorbance at 450 nm. The yellow colour indicates the presence of Strep-HRP, which in turn indicates the presence of aptamers bound to either the well or the immobilised protein.

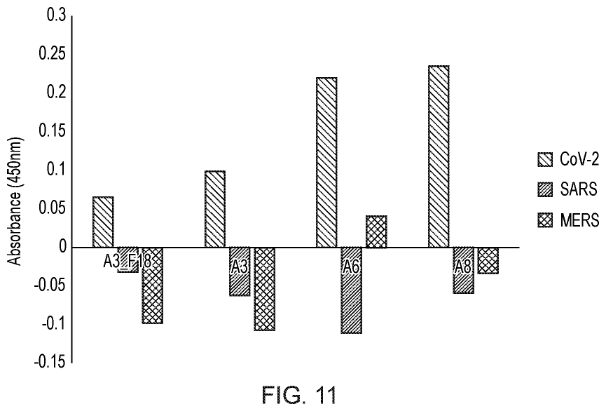

Absorbance data was background corrected by subtracting the ‘no protein’ control wells, from each of the corresponding wells; to leave only the response which is due to aptamer bound to immobilised protein. Corrected data was plotted for each of the aptamers and each of the S1 subunits of the spike protein from the respective virus (FIG. 11). Absorbance values of 0 indicate that the background signal was higher than the protein bound signal; indicating no aptamer interaction. The results show that each of the aptamers interacts with the S1 subunit from the spike protein from SARS-CoV-2 (left bars) but not with the corresponding proteins from SARS or MERS (middle and right bars respectively). This demonstrates both the specificity of the aptamers and indicates their performance in an indirect ELONA.

### Example 6—Optimers Isolated Against the S2 Subunit of the SARS-CoV-2 Spike Protein, Bind to Full Spike Protein from SARS-CoV-2

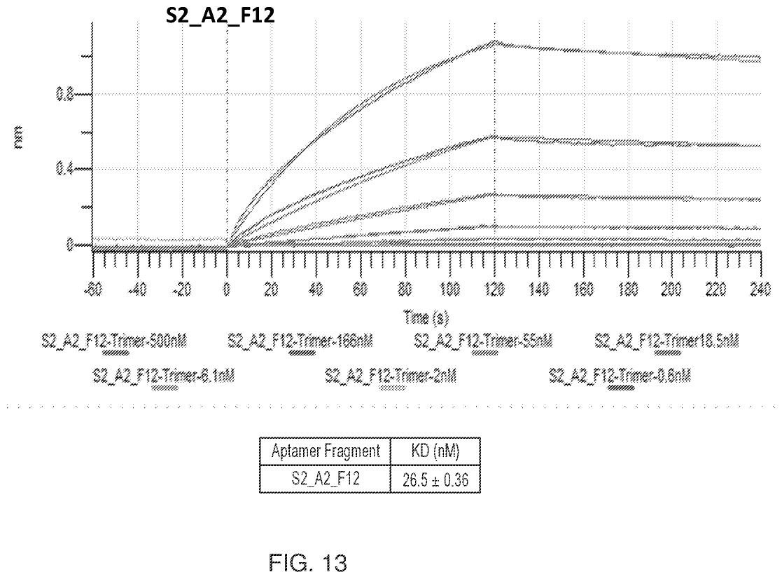

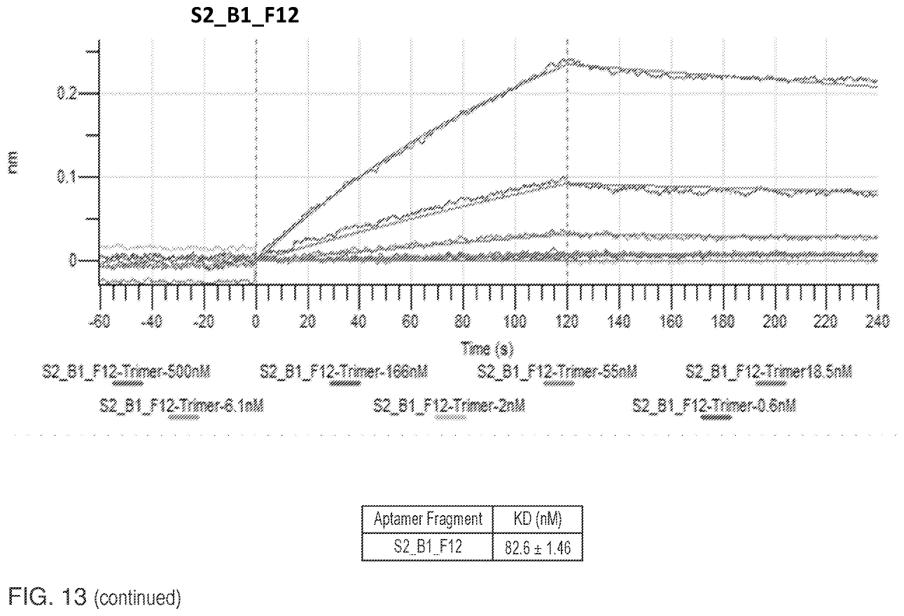

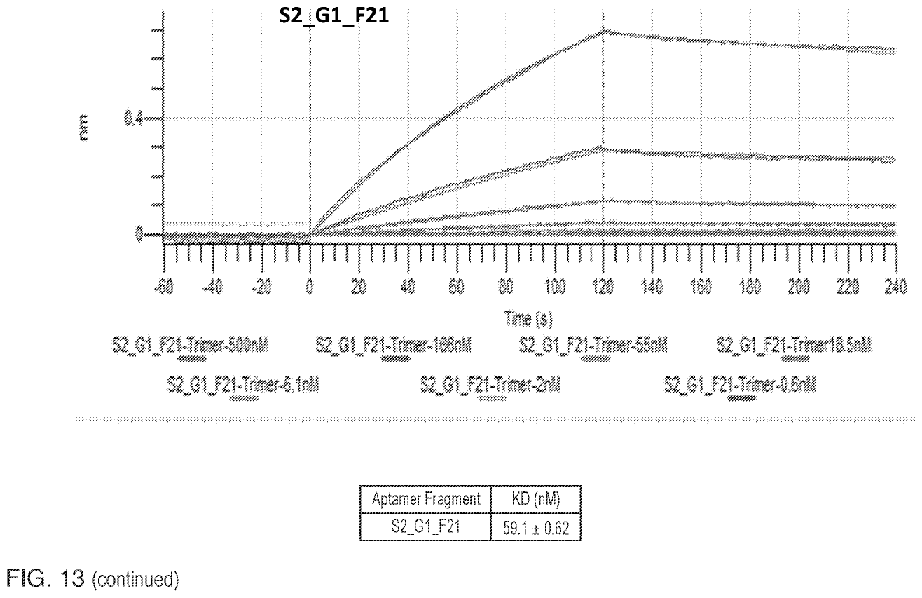

The aptamers described herein have been isolated using recombinant proteins which represent portions of the full viral surface spike protein (SEQ ID NO: 117). In particular, an amino acid sequence comprising residues Ser686 to Pro1213 of SEQ ID NO: 119 was used to isolate aptamers against the S2 subunit from the SARS-CoV-2 spike protein. For diagnostic and therapeutic applications of the aptamers, it is desirable that the aptamers are capable of binding to the spike protein in the full trimeric form. For example, any aptamers which bind to regions of the monomer which are not accessible in the trimeric spike may not be capable of binding to the virus and so would be less likely to perform well as a diagnostic or therapeutic. Several of the isolated aptamers and Optimers isolated against the S2 subunit of the SARS-CoV-2 spike protein (SEQ ID NO:119), were assessed for binding to the full trimeric spike using the BLI assay described above. Briefly, exemplar Optimers were immobilised onto BLI sensor probes and assessed for the ability to bind to the full SARS-CoV-2 spike protein trimer at a range of concentrations (500 nM, 166 nM, 55 nM, 18.5 nM, 6.1 nM, 2 nM and 0.6 nM in 1× ‘high salt’ aptamer selection buffer). Data shown in FIG. 13 demonstrates a clear concentration dependant interaction between the immobilised aptamer and spike protein trimer; indicating that each of the aptamers have high affinity (˜26-82 nM) for the full spike protein. Importantly, each of the S2_A2_F12 SEQ ID NO: 179), S2_B1_F12 SEQ ID NO: 188) and S2_G1_F21 (SEQ ID NO: 190) aptamers has a rapid association rate (0-120 sec) and a slow dissociation rate (120-240 sec); which may be important for many diagnostic applications.

An overview of the various properties of the best performing S2 subunit binding aptamers and fragments thereof is shown in Table 5 below:

| SEQ ID NO: | Clone Name | S2 | Trimer binding |
|---|---|---|---|
| 146 | S2_A2 | ++ | |
| 150 | S2_B1 | ++ | |
| 171 | S2_G1 | ++ | |
| 177 | S2_A2_F17 | ++ | + |
| 179 | S2_A2_F12 | ++ | ++ |
| 183 | S2_B1_F18 | ++ | + |
| 188 | S2_B1_F12 | ++ | ++ |
| 190 | S2_G1_F21 | ++ | ++ |
| 191 | S2_G1_F22 | ++ | + |

### Example 7—Optimers Isolated Against the S1 Subunits of the SARS-CoV-2 Spike Protein, Bind to the S1 Domain of the Spike Protein from Wild-Type SARS-CoV-2 and Variants of Concern

The aptamers described herein have been isolated using recombinant proteins which represent portions of the full viral surface spike protein from SARS-CoV-2 (SEQ ID NO: 117). For diagnostic and therapeutic applications of the aptamers, it is desirable that the aptamers and/or minimal functional fragment Optimers are capable of binding to the spike protein from the ‘original’ Wild-type form of SARS-CoV-2 and all major ‘variants of concern’. For example, diagnostic device which is based on a pair of aptamers or Optimers which only bind to the ‘wild-type form’ of SARS-CoV-2, but do not recognise the variants of concern; will likely give a ‘false negative’ test result if the sample is taken from an individual who is infected with a COVID-19 variant. The aptamers and Optimers must therefore recognise all variants of the SARS-CoV-2 spike protein.

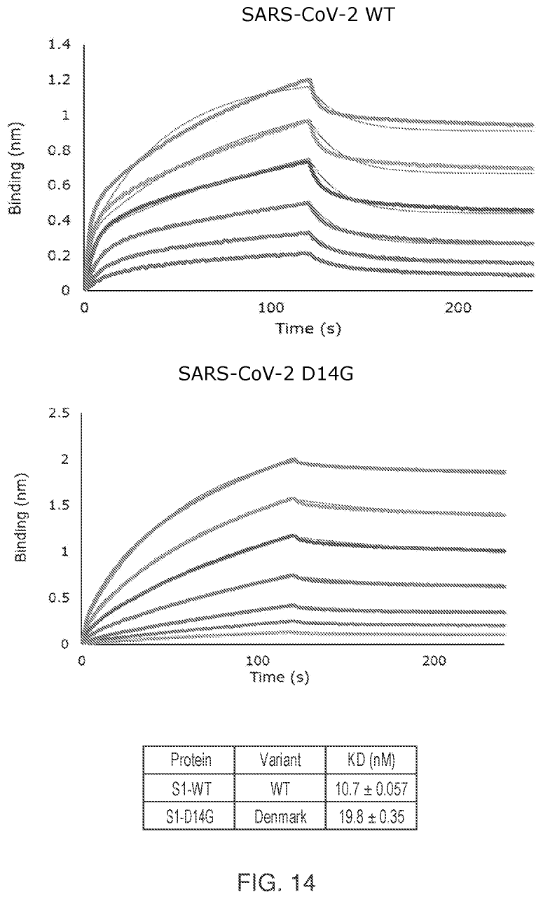

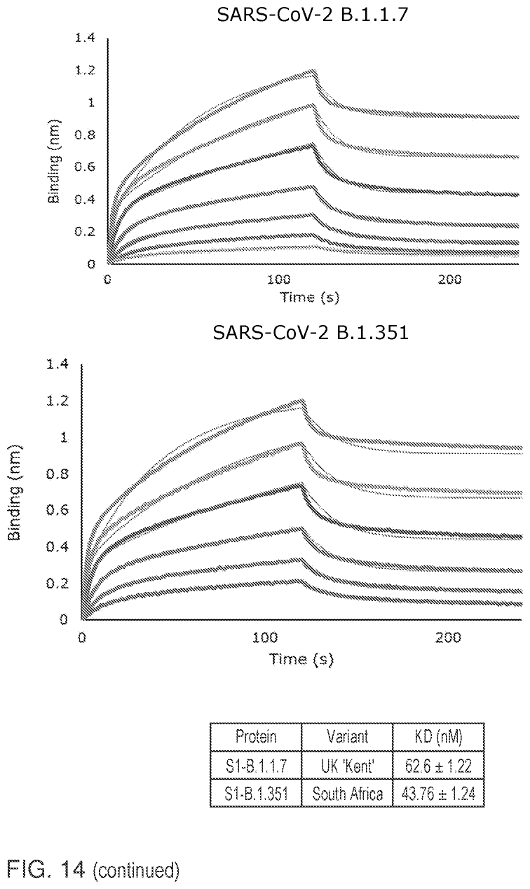

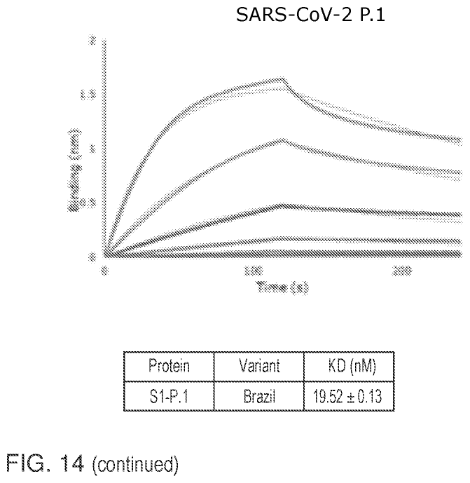

The preferred Optimer S1_A8_F21 (SEQ ID NO: 140) isolated against the S1 subunit of the SARS-CoV-2 spike protein, was assessed for binding to the S1 subunit of the spike protein from ‘wild-type’ SARS-CoV-2, as well as the UK ‘Kent variant’ (B.1.1.7), ‘Denmark variant’ (D14G), ‘South Africa variant’ (B.1.351), and the ‘Brazil variant’ (P.1) using the BLI assay described above. Briefly, the exemplar Optimer was immobilised onto BLI sensor probes and assessed for the ability to bind to the S1 subunit of the spike protein from each of the variants of SARS-CoV-2, at a range of concentrations (500 nM, 166 nM, 55 nM, 18.5 nM, 6.1 nM, 2 nM and 0.6 nM in 1× ‘high salt’ aptamer selection buffer). Data shown in FIG. 14 demonstrates a clear concentration dependant interaction between the immobilised Optimer and the S1 subunit of the spike protein from each of the variants of SARS-CoV-2; indicating that the Optimer recognises the wild-type form of SARS-CoV-2 and each of the variants of concern, with high affinity (˜10-62 nM). Importantly, the Optimer has a rapid association rate (0-120 sec) and a slow dissociation rate (120-240 sec); which may be important for many diagnostic applications and recognises all of the variants tested here; which is essential for a viable diagnostic product. Other variants of concern will be tested as they arise, and their respective S1 subunit proteins become available.

### Example 8—Optimers Isolated Against the S1 or S2 Subunits of the SARS-CoV-2 Spike Protein, Bind Irradiated Virus Material from Wild-Type SARS-CoV-2 and Variants of Concern

The aptamers described herein have been isolated using recombinant proteins which represent portions of the full viral surface spike protein from SARS-CoV-2 (SEQ ID NO: 117). For diagnostic and therapeutic applications of the aptamers, it is desirable that the aptamers and/or minimal functional fragment Optimers are capable of binding to the ‘original’ Wild-type form of SARS-CoV-2 and all major ‘variants of concern’. For example, diagnostic device which is based on a pair of aptamers or Optimers which only bind to the ‘wild-type form’ of SARS-CoV-2, but do not recognise the variants of concern; will likely give a ‘false negative’ test result if the sample is taken from an individual who is infected with a COVID-19 variant. The aptamers and Optimers must therefore recognise all variants of the SARS-CoV-2. It is also important that the Optimers recognise the respective region of the protein as it is presented on the surface of the virus.

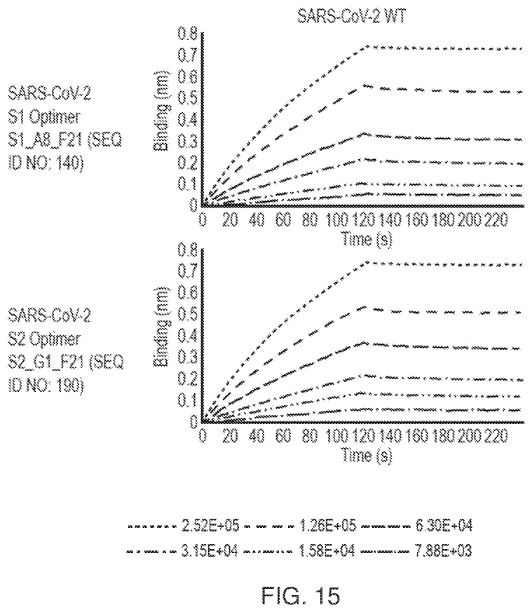

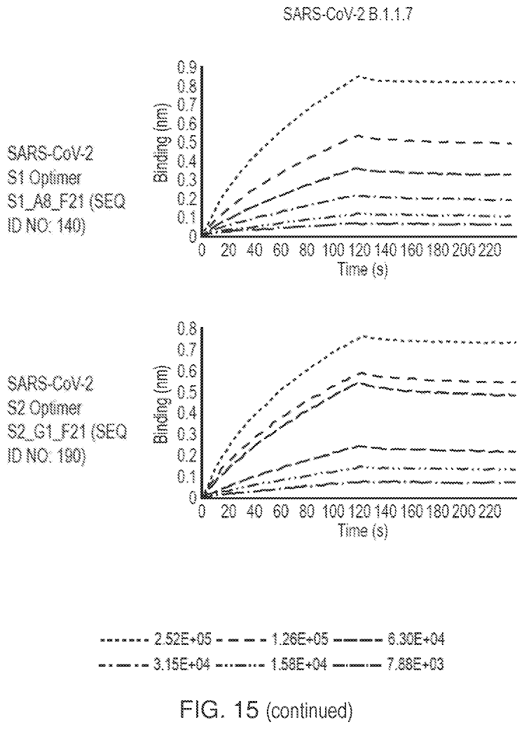

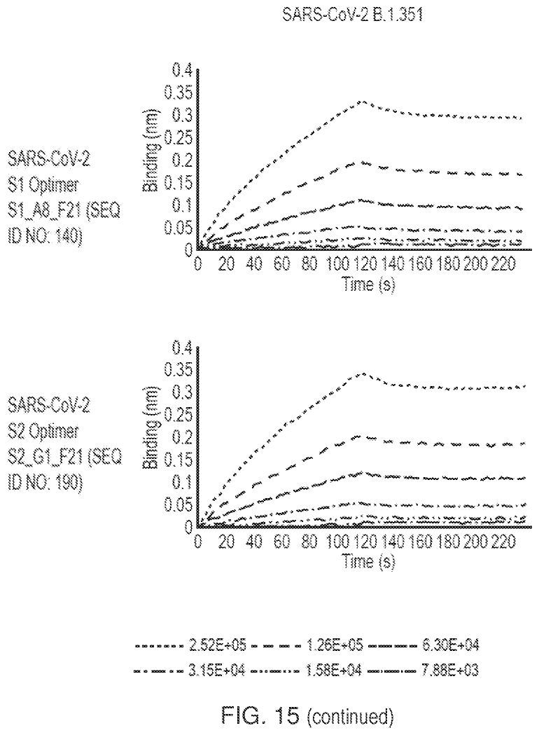

The preferred Optimers S1_A8_F21 (SEQ ID NO: 140) and S2_G1_F21 (SEQ ID NO: 190) isolated against the S1 and S2 subunit of the SARS-CoV-2 spike protein respectively, were assessed for binding to irradiated cultured viral material from ‘wild-type’ SARS-CoV-2, as well as the UK ‘Kent variant’ (B.1.1.7) and the ‘South Africa variant’ (B.1.351) using the BLI assay described above. Briefly, the exemplar Optimers were immobilised onto BLI sensor probes and assessed for the ability to bind to the irradiated viral material from each of the variants of SARS-CoV-2, at a range of concentrations (2.52e5, 1.26e5, 6.30e4, 3.15e4, 1.58e4 and 7.88e3 plaque forming units (pfu) per ml in 1× ‘high salt’ aptamer selection buffer). Data shown in FIG. 15 demonstrates a clear concentration dependant interaction between the immobilised Optimer and the S1 subunit of the spike protein from each of the variants of SARS-CoV-2; indicating that the Optimer recognises the wild-type form of SARS-CoV-2 and each of the variants of concern. Importantly, both of the Optimers have a rapid association rate (0-120 sec) and a slow dissociation rate (120-240 sec); which may be important for many diagnostic applications and recognise all of the variants tested here; which is essential for a viable diagnostic product. Other variants of concern will be tested as they arise, and irradiated viral material becomes available.

### Example 9—Identification of Non-Competing Aptamer Pairs which Bind to the S1 or S2 Subunits of the SARS-CoV-2 Spike Protein (Optimer Sandwich ELONA)

Sandwich ELONA assays are generally preferred to other assays in which a single affinity ligand is used (e.g., direct or indirect ELONA) as the use of two affinity ligands generally leads to improved specificity.

In this example, an identified S1 binding aptamer A3-A (SEQ ID NO: 4); or minimal functional fragment thereof, A3_F18 (SEQ ID NO: 134), were prepared with a 5′ biotin tag. The biotin tag was used to immobilise the aptamers/Optimers onto all wells of separate streptavidin coated ELISA plates (StreptaWell plates, Roche). After removing the unbound aptamer and blocking the unbound binding sites (with 1 mM biotin in 1×PBS, 0.01% (v/v) Tween-20); the plates were incubated with a gradient of SARS-CoV-2 Spike protein trimer (15.6-1000 ng/ml).

A row of wells on each plate, was left blank (no protein) to allow background subtraction of non-specific aptamer binding. After incubation (90 mins at room temperature); the unbound protein was removed, and the wells washed thoroughly with 1× ‘high salt’ aptamer selection buffer.

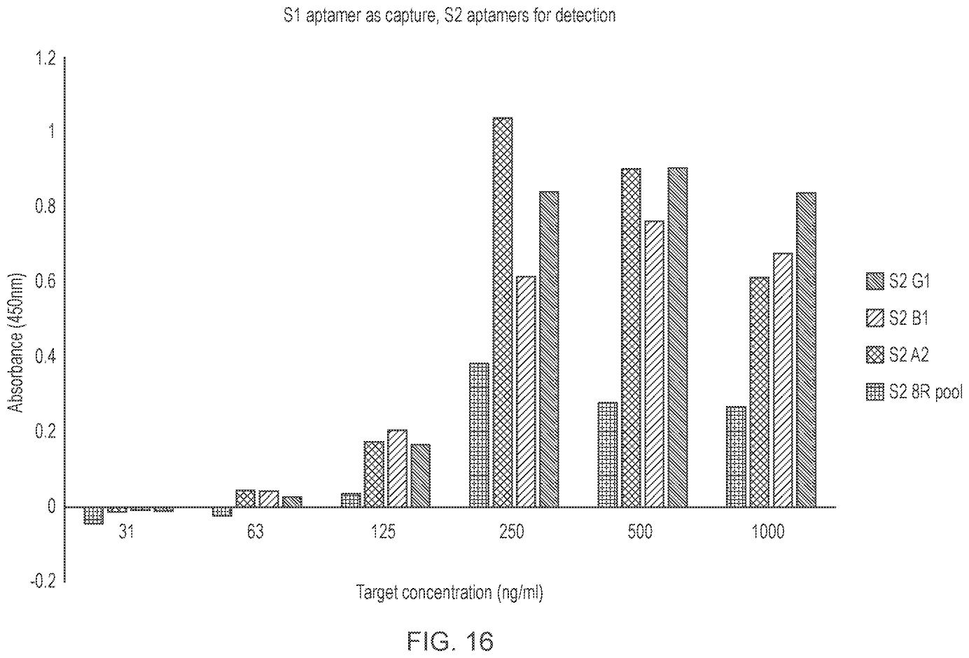

One ELONA plate (immobilised with the S1 binding aptamer A3) was then incubated with a fixed concentration (0.5 μM) of either the aptamer population isolated against the S2 subunit of the SARS-CoV-2 spike protein (S2_8R pool) or full-length aptamer clones S2_A2 (SEQ ID NO: 146), S2_B1 (SEQ ID NO: 150) and S2_G1 (SEQ ID NO: 171) (FIG. 16).

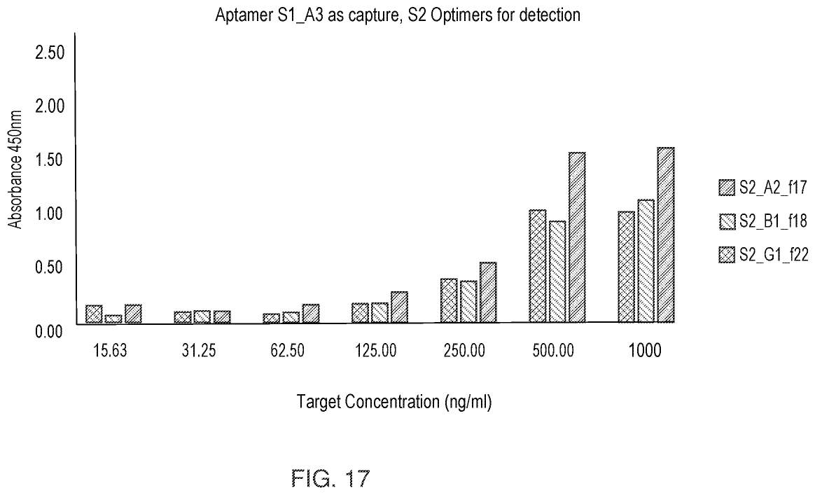

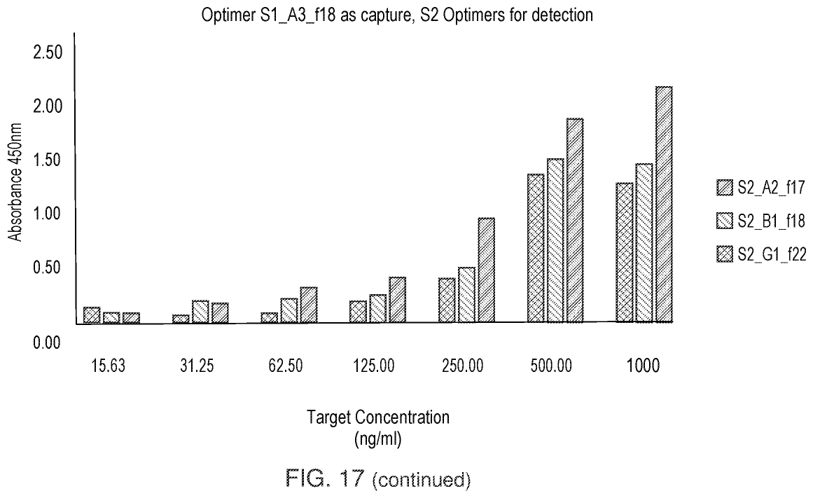

Two other ELONA plates (one immobilised with S1 binding aptamer A3; the other with S1 binding Optimer A3_F18) were incubated with a fixed concentration (0.5 μM) of the minimal functional fragments S2_A2_F17, S2_B1_F18 or S2_G1_F22 (SEQ ID NO: 177, 183 and 191 respectively) (FIG. 17).

After incubating the three plates listed above (90 mins at room temperature); unbound aptamer was removed by washing the wells with 1× ‘high salt’ aptamer selection buffer. Wells were then incubated with strep-HRP (0.2 μg/ml in 1×PBS) to capture the enzyme on any protein bound aptamers. After washing wells to remove unbound Strep-HRP; each of the wells was incubated with the substrate, 3,3′, 5,5′-tetramethylbenzidine (TMB) which yields a blue colour when cleaved by HRP. Termination of the enzymatic activity (with 1M HCl) yields a yellow product which can be quantified by measuring the absorbance at 450 nm. The yellow colour indicates the presence of Strep-HRP, which in turn indicates the presence of aptamers bound to either the well or the immobilised protein.

Absorbance data was background corrected by subtracting the ‘no protein’ control wells, from each of the corresponding wells; to leave only the response which is due to aptamer bound to immobilised protein. Corrected protein concentration dependent data was plotted for each of the Aptamers/Optimers. Absorbance values of ≤0 indicate that the background signal was higher than the protein bound signal; indicating no aptamer interaction.

The results in FIG. 16 show that each of the S2 binding aptamers (S2_A2, S2_B1 and S2_G1; SEQ ID NO: 146, 150 and 171 respectively) is able to bind and detect a concentration series of the SARS-CoV-2 spike protein trimer when it is captured on plates immobilised with the full-length S1 binding aptamer, A3 (SEQ ID NO: 4). This shows that each of these full-length aptamers can be used in a sandwich ELONA and similar diagnostic platforms e.g., Lateral Flow Devices (LFD).

The results in FIG. 17 show that each of the S2 binding Optimers (S2_A2_F17, S2_B1_F18 and S2_G1_F22; SEQ ID NO: 177, 183 and 191 respectively) is able to bind and detect a concentration series of the SARS-CoV-2 spike protein trimer when it is captured on plates immobilised with either the full-length S1 binding aptamer, A3 (SEQ ID NO:4) or the minimal functional S1 binding Optimer, A3_F18 (SEQ ID NO: 134). This shows that each of these minimal functional Optimers can be used in a sandwich ELONA with either the full-length S1 aptamer or the corresponding minimal functional fragment.

In these examples, it has been shown that the S1 binding Aptamer(s) and Optimer(s) may be used as a ‘capture reagent’ and the S2 binding Aptamer(s) and Optimer(s) may be used as a ‘detection reagent’ in a sandwich ELONA or similar assay. It is expected that the Aptamer(s) and Optimer(s) may be in the other orientation i.e., S2 ‘capture’ and S1 ‘detection’.

### Example 10—Optimers Isolated Against the S1 or S2 Subunits of the SARS-CoV-2 Spike Protein, Bind Irradiated Virus Material from Wild-Type SARS-CoV-2 in a Simple Lateral Flow Device Assay Format

Lateral Flow Devices are simple test formats that can be run without the need for special laboratory equipment. They are a preferred assay format for simple, rapid mass screening of large numbers of samples. In this test format, one of the binding pair of Optimers is immobilised on the ‘test line’, the other Optimer is immobilised onto the surface of a Gold Nanoparticle and deposited in the ‘conjugate pad’. When the test is run, a sample is applied to the ‘sample pad’. The material runs along the lateral flow membrane by capillary action. If the virus is present in the sample, it is captured on the immobilised Optimer in the ‘test line’. Optimer coated Gold Nanoparticles are detector particles that bind to virus captured on the ‘test line’ to give a visible signal. In the absence of the virus, the Optimer coated Gold Nanoparticles are not captured on the ‘test line’, giving a ‘blank’ test line. A ‘control line’ is used to show that the test has run correctly. In this case the ‘control line’ consists of an immobilised ‘capture oligonucleotide’ that is complimentary to the Optimer on the Gold Nanoparticles. Hybridization between the immobilised ‘capture oligonucleotide’ and the Optimer on the Gold Nanoparticles leads to the formation of a ‘control line’.

The aptamers described herein have been isolated using recombinant proteins which represent portions of the full viral surface spike protein from SARS-CoV-2. The preferred Optimers S1_A8_F21 (SEQ ID NO: 140) and S2_G1_F21 (SEQ ID NO: 190) are isolated against the S1 and S2 subunit of the SARS-CoV-2 spike protein respectively.

Preparation of the Gold Nanoparticles

In this example the preferred Optimer S2_G1_F21 (SEQ ID NO: 190) isolated against the S2 subunit of the SARS-CoV-2 spike protein, was prepared with a 5′ thiol group to allow the Optimer to be immobilised onto 40 nm OD5 Gold Nanoparticles (BBI Solutions, UK). Briefly, 1 ml of OD5 gold colloid was transferred into a 1.5 ml Eppendorf tube and centrifuged for 10 minutes at 4000×g to pellet the Gold Nanoparticles. The 500 μl of supernatant was removed, and the pellet resuspend in remaining 500 μl of solution to prepare OD10 nanoparticle stock. 0.5 mg of aptamer stock was applied directly to 1 ml of OD10 gold colloid stock solution and pipette mixed prior to incubating for 1 minute at room temperature on a shaker (Heidolph, Titramix 100, S/N 544-11200-00-3). 25 μl of 0.5M Tri-Sodium Citrate was added to the gold/aptamer solution, mixed gently by pipetting, and incubated for a further 10 minutes at room temperature. 17.5 μl of 2M Sodium chloride was added to the aptamer/gold solution slowly (dropwise) and mixed gently by pipetting, then incubated for a further 20 minutes at room temperature on a plate shaker to agitate the mix. 50 μl of 2M Sodium Chloride was added to the aptamer/gold solution slowly (dropwise) and mixed again by gentle pipetting, then incubated for a further 40 minutes at room temperature on a plate shaker to agitate the mix. The aptamer conjugated gold colloid was then centrifuged at 4000×g for 15 minutes to form a pellet. The supernatant was carefully removed and discarded, and the pellet was resuspended with 500 μl of 1× Phosphate Buffered Saline (PBS) with 0.05% Tween-20. The aptamer/gold mixture was centrifuged again at 4000×g for 15 minutes to re-form the pellet. The supernatant was removed, and the pellet resuspended in 500 μl of 1×PBS supplemented with 1 mM Magnesium Chloride. The aptamer/gold mixture was pelleted a final time at 4000×g for 15 minutes and the pellet was resuspended in 333 μl 20 mM Taps (pH 7.5), 5% sucrose, 3% BSA, 1% Tween-20 (Gold drying buffer) to prepare final gold at OD15. To spray the isoflow dispenser (Imagene technologies S/N 120731/108363) set up using the following parameters: Nozzle height (from conjugate pad) at 15 mm, dispense rate of 0.6 μl/ml across a 30 cm band and a spray pressure of 5 PSI. The sprayed conjugate pad dried by passing through a drying tunnel (Hedinar, S/N 0596109) at 5 mm/c at 60° C. All pads were stored within a heat-sealed foil pouch containing desiccant until required for assembly.

Preparation of the Derivatised Nitrocellulose Membrane

Preparation of both test and control line aptamers follow the same initial procedure. The preferred S1 binding Optimer S1_A8_F21 (SEQ ID NO: 140) and ‘capture oligonucleotide’ (SEQ ID NO: 193) were each synthesised with a 5′ biotin group. A separate mix was prepared for each oligonucleotide, containing 33.4 μM of the respective oligonucleotide and 1 mg/ml polystreptavidin (BioTez Berlin-Buch Gmbh, cat #10312116) e.g., for a 200 μl final volume of ‘test line’ or ‘control line’ stock; 55.6 μl of 120 μM biotinylated oligonucleotide stock was combined with 87.3 μl 1× Phosphate Buffered Saline and 57.1 μl of 3.5 mg/ml polystreptavidin stock. The components were combined by pipette mixing and incubated without additional mixing at room temperature for 1 hour. The isoflow dispenser (Imagene technologies S/N 120731/108363) was set up using the following parameters: ‘test line’ dispense nozzle to 7 mm and ‘control line’ dispense nozzle to 13 mm. Both lines were dispensed at half rate (0.05 μl/mm) across a 30 cm length band. The plotted NC was marked to denote the start and end of plotted lines and any potential inconstant plotting along the length of the band. The membrane was then dried by passing through a drying tunnel (Hedinair, S/N 0596109) at 10 mm/sec at 60° C. All membranes were stored within a heat-sealed foil pouch containing desiccant until required for assembly.

Strip Lamination

The gold conjugate pad (17 mm width) was prepared with S2 aptamer colloid (BBI™ Solutions, cat #EM/GC40). The tape cover was removed from backing card (single side adhesive (Kenosha, KN-PS1060.197, S/N 45253420) and the laminator height was set to allow a 20 mm gap from the bottom of the backing card to the bottom of the NC. The NC was placed with the ‘test line’ in the lower most position. The 22 mm wide ‘sink pad’ (Ahlstrom, grade 222 22 mm×100 m) was positioned against the top edge of the backing card such that a 7 mm overlap with the NC was created (see diagram below). The ‘Conjugate pad’ was placed 5 mm from the bottom of the backing card, to create a 2 mm overlap with the NC (see diagram below). The glass fibre ‘Sample pad’ (Ahlstrom, grade 8951 12 mm×100 m) was then aligned with the bottom of the backing card, to create a 7 mm overlap with ‘Conjugate pad’ (see diagram below). The band was then laminated and cut into individual 5 mm strips and assembled into standard single sample well housing.

Running the Test Assay

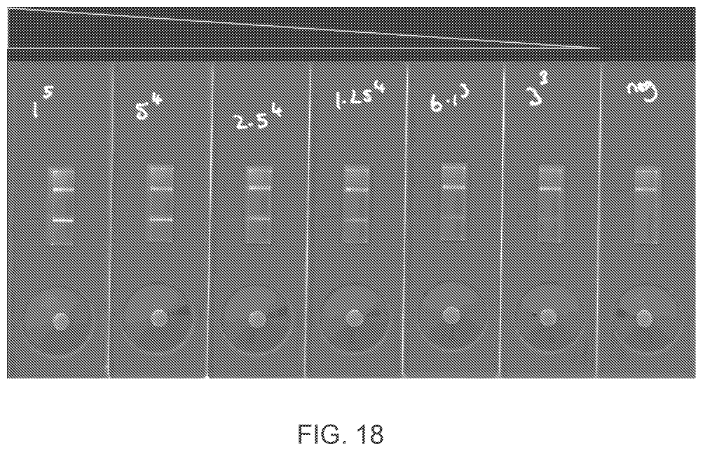

In the demonstrator test (FIG. 18), a dilution series of virus material was prepared at a range of concentrations (1×10\hat{ }5, 5×10\hat{ }4, 2.5×10\hat{ }4, 1.25×10\hat{ }4, 6.1×10\hat{ }3 and 3×10\hat{ }3 pfu/mi; and a negative control sample with no virus material) in extraction buffer (mentioned above) from a master stock of 6.3×10\hat{ }5 pfu/mi irradiated viral particles. 80 μl of each sample in the dilution series was pipetted onto a separate device. The test was then left to run for 10 minutes. Any readings after this time are classified as invalid and ensure a control line has formed within the running time.

The results presented in FIG. 18 clearly show the presence of a ‘Control line’ (upper band) in every test (including the negative control, ‘neg’), indicating that the tests have all run successfully. The ‘Test line’ (lower band) shows a clear concentration dependent response. A feint band is seen in the 3×10\hat{ }3 test indicating a lower limit of detection in the region of ˜1-5×10\hat{ }3 pfu/ml.

Running the Test Assay—Clinical Samples

Prior to sample collection, the donor was asked to gently blow their nose, then gently swab the anterior nasal cavity with the sterile swab (Miraclean, sterile foam tipped, 93050L) (˜5 rotations per nostril). The swab was immediately placed in the extraction tube containing 400 μl extraction buffer (1M NaCl, 300 mM Tris, 0.1% Tween-20, 0.2% BSA, 1 mM MgCl₂, 0.05% NaN₃ pH 8.5) and rotated back and forth 10 times. The swab was gently squeezed when withdrawing from the extraction tube to maximise the sample recovery. The cap was closed on the extraction tube before inverting it for ˜5 seconds to allow sample to pool into the filter in the dropper nozzle. The bottle was then gently squeezed to slowly add 3 drops of sample into the sample well of the Lateral Flow Device. Care was taken to ensure that the sample was taken into the device in the first 10 seconds of application. If it did not run, a fourth drop was added. The test was then left to run for 10 minutes. Any readings after this time are classified as invalid and ensure a control line has formed within the running time.

The reader's attention is directed to all papers and documents which are filed concurrently with or previous to this specification in connection with this application and which are open to public inspection with this specification, and the contents of all such papers and documents are incorporated herein by reference.

1.-27. (canceled)

28. One or more aptamers capable of specifically binding to the S1 subunit of the SARS-CoV-2 spike protein, the S2 subunit of the SARS-CoV-2 spike protein, or both, wherein the one or more aptamers comprise:
(a) a nucleic acid sequence selected from any one of SEQ ID NOs: 4, 8, 9 or 43;
(b) a nucleic acid sequence selected from any one of SEQ ID NOs: 10, 20, 24 or 44;
(c) a nucleic acid sequence selected from any one of SEQ ID NOs: 134, 140 or 144;
(d) a nucleic acid sequence selected from any one of SEQ ID NOs: 146, 150 or 171;
(e) a nucleic acid sequence selected from any one of SEQ ID NOs: 177, 179, 183, 188, 190 or 191;
(f) a nucleic acid sequence having at least about 15 consecutive nucleotides of any one of the sequences of (a) to (e); and/or
(g) a nucleic acid sequence having at least about 85% or more sequence identity with any one of the sequences of (a) to (f).

29. The one or more aptamers of claim 28, wherein the aptamers comprise:
(a) a nucleic acid sequence capable of specifically binding to the S1 subunit of the SARS-CoV-2 spike protein, wherein the sequence is selected from SEQ ID NO: 140;
(b) a nucleic acid sequence capable of specifically binding to the S2 subunit of the SARS-CoV-2 spike protein, wherein the sequence is a nucleic acid sequence selected from SEQ ID NO: 190;
(c) a nucleic acid sequence having at least about 25 consecutive nucleotides of the sequences of (a) or (b); and/or
(d) a nucleic acid sequence having at least about 85% or more sequence identity with any one of the sequences of (a) to (c).

30. The one or more aptamers of claim 28, comprising:
(a) a first aptamer comprising a nucleic sequence selected from any one of SEQ ID NOs: 4, 8, 9, 43, 134, 10 or 144 or a sequence having at least about 90% or more sequence identity with any one of SEQ ID NOs: 4, 8, 9, 43, 134, 140 or 144; and
(b) (i) a second aptamer comprising a nucleic sequence selected from any one of SEQ ID NOs: 4, 8, 9, 43, 134, 140 or 144 or a sequence having at least about 90% or more sequence identity or more with any one of SEQ ID NOs: 4, 8, 9, 43, 134, 140 or 144, wherein the second aptamer is different than the first aptamer; or
(ii) a second aptamer comprising a nucleic sequence selected from any one of SEQ ID NOs: 146, 150, 171, 177, 179, 183, 188, 190 or 191 or a sequence having at least about 90% or more sequence identity with any one of SEQ ID NOs: 146, 150, 171, 177, 179, 183, 188, 190 or 191.

31. The one or more aptamers of claim 30, wherein:
(a) the first aptamer comprises a nucleic sequence selected from SEQ ID NO: 140 or a sequence having at least about 95% or more sequence identity with SEQ ID NO: 140; and
(b) the second aptamer comprises a nucleic sequence selected from SEQ ID NO: 190 or a sequence having at least about 85% or more sequence identity with SEQ ID: NO 190.

32. The one or more aptamers of claim 28, wherein:
(i) the aptamer is a DNA aptamer; or
(ii) the one or more aptamers comprise a detectable label selected from a fluorophore, a nanoparticle, a quantum dot, an enzyme, a radioactive isotope, a pre-defined sequence portion, a biotin, a desthiobiotin, a thiol group, an amine group, an azide, an aminoallyl group, a digoxigenin, an antibody, a catalyst, a colloidal metallic particle, a colloidal non-metallic particle, an organic polymer, a latex particle, a nanofiber, a nanotube, a dendrimer, a protein, and a liposome.

33. An aptamer that competes for binding to the S1 and/or S2 subunits of the SARS-CoV-2 spike protein with the one or more aptamers of claim 28.

34. A complex comprising the one or more aptamers of claim 28 and a detectable molecule.

35. The complex of claim 34, further comprising the S1 subunit of the SARS-Cov-2 spike protein, the S2 subunit of the SARS-Cov-2 spike protein, a monomer of the SARS-CoV-2 spike protein, a trimer of the SARS-CoV-2 spike protein, the SARS-Cov-2 virus, or combinations of any of the foregoing.

36. A biosensor, assay plate or test strip comprising the one or more aptamers of claim 28.

37. An apparatus for detecting the presence, absence or level of SARS-CoV-2 in a sample, comprising: the one or more aptamers of claim 28 and a support.

38. A lateral flow device comprising the one or more aptamers of claim 28.

39. The lateral flow device of claim 38, further comprising:
(a) a first aptamer comprising a nucleic sequence selected from SEQ ID NO: 140 or a sequence having at least about 85% or more sequence identity with SEQ ID NO: 140; and
(b) a second aptamer comprising a nucleic sequence selected from SEQ ID NO: 190 or a sequence having at least about 85% or more sequence identity with SEQ ID NO: 190.

40. The lateral flow device of claim 39, wherein either the first aptamer or the second aptamer is bound to a solid support.

41. The lateral flow device of claim 40, wherein:
(i) if the first aptamer is bound to the solid support, the second aptamer is conjugated to a detectable label; or
(ii) if the second aptamer is bound to the solid support, the first aptamer is conjugated to a detectable label;
wherein the detectable label is a gold nanoparticle, a latex nanoparticle, a fluorescent nanoparticle, a fluorophore or a Quantum Dot.

42. A functionalised electrode or biosensor surface comprising the one or more aptamers of claim 28.

43. The functionalised electrode or biosensor of claim 42, wherein the one or more aptamers are:
(a) an aptamer comprising a nucleic sequence selected from SEQ ID NO: 10, 20, 24 or 44; or
(b) a sequence having at least about 85% or more sequence identity with SEQ ID NO: 10, 20, 24 or 44;
wherein the aptamer is immobilised onto the surface of the electrode.

44. A method of detecting the presence, absence or amount of SARS-CoV-2 in a sample, comprising:
(i) interacting the sample with the one or more aptamer(s) of claim 28; and
(ii) detecting the presence, absence or amount of SARS-CoV-2.

45. The method of claim 45, wherein:
(i) the sample is obtained from a subject having or suspected of having a COVID-19 infection;
(ii) the sample is saliva, blood, or a nasal swab; or
(iii) an infecting viral load of SARS-CoV-2 is detected.

46. A kit comprising: the one or more aptamers of claim 28; and a linker, support, detectable molecule, or combination thereof, wherein the kit detects, quantifies or enriches SARS-CoV-2.


SEQUENCE LISTING (193 sequences)

SEQ ID NO: 1 - DNA, 82 bp, Artificial Sequence
  S1 Aptamer A1-A (S1_A0
ccagtgtagactactcaatgccgatgggtcgggtgggtgggtaggcattgatcgctccttccgtactatccacaggtcaacc

SEQ ID NO: 2 - DNA, 81 bp, Artificial Sequence
  S1 Aptamer A1-B (S1_A1-B)
ccagtgtagactactcaatgcggctgtgtgacttgacctctggatatgggagggagggcgggtactatccacaggtcaacc

SEQ ID NO: 3 - DNA, 81 bp, Artificial Sequence
  S1 Aptamer A2 (S1_A2)
ccagtgtagactactcaatgcttcggcgctcctaattttatcccctagattcatattttccgtactatccacaggtcaacc

SEQ ID NO: 4 - DNA, 81 bp, Artificial Sequence
  S1 Aptamer A3-A (S1_A3-A)
ccagtgtagactactcaatgcgtgggaagggtgggtgggagcattgataactcggaggagtgtactatccacaggtcaacc

SEQ ID NO: 5 - DNA, 82 bp, Artificial Sequence
  S1 Aptamer A3-B (S1_A3-B)
ccagtgtagactactcaatgccgatgggtcgggggggtgggtaggcattgatcgctcctttcgtactatccacaggtcaacc

SEQ ID NO: 6 - DNA, 81 bp, Artificial Sequence
  S1 Aptamer A4 (S1_A4)
ccagtgtagactactcaatgcgtgggaagggtgggcgggagcattgataactcggaggagcgtactatccacaggtcaacc

SEQ ID NO: 7 - DNA, 81 bp, Artificial Sequence
  S1 Aptamer A5 (S1_A5)
ccagtgtagactactcaatgcggtgactgctgagatagacagagtccgaggaccgtttaaagtactatccacaggtcaacc

SEQ ID NO: 8 - DNA, 81 bp, Artificial Sequence
  S1 Aptamer A6 (S1_A6)
ccagtgtagactactcaatgctgttatgacttgaccttgggttatgggtttgggcgggagggtactatccacaggtcaacc

SEQ ID NO: 9 - DNA, 82 bp, Artificial Sequence
  S1 Aptamer A8 (S1_A8)
ccagtgtagactactcaatgctgtgttgacttgatcctgtggtatatgggtgggagggtcgggtactatccacaggtcaacc

SEQ ID NO: 10 - DNA, 82 bp, Artificial Sequence
  S1 Aptamer A9 (S1_A9)
ccagtgtagacttacacctgcagtgttcacgagggccggcggtatatgggcggttgatacgagtactatccacaggtcaacc

SEQ ID NO: 11 - DNA, 85 bp, Artificial Sequence
  S1 Aptamer A12 (S1_A12)
ccagtgtagactactcaatgccgtgtggtgaccttgatcctgtggtatatgggtgggaggggcgggtactatccacaggtcaacc

SEQ ID NO: 12 - DNA, 81 bp, Artificial Sequence
  S1 Aptamer B1 (S1_B1)
ccagtgtagactactcaatgccgatgggtcgggtgggtgggtaggcattgatcgctcctcagtactatccacaggtcaacc

SEQ ID NO: 13 - DNA, 82 bp, Artificial Sequence
  S1 Aptamer B3 (S1_B3)
ccagtgtagactactcaatgccgatgggtcgggggggtgggtaggcattgattcctcctttcgtactatccacaggtcaacc

SEQ ID NO: 14 - DNA, 81 bp, Artificial Sequence
  S1 Aptamer B4 (S1_B4)
ccagtgtagactactcaatgctgtgttgacttgatcctgtggtatatgggtgggagggagggtactatccacaggtcaacc

SEQ ID NO: 15 - DNA, 81 bp, Artificial Sequence
  S1 Aptamer B8 (S1_B8)
ccagtgtagactactcaatgctgagatgacttgatcctgtcgtagatggggggaaggtcgggtactatccacaggtcaacc

SEQ ID NO: 16 - DNA, 81 bp, Artificial Sequence
  S1 Aptamer B9 (S1_B9)
ccagtgtagactactcaatgctgtgttgacttgatcctgtggtatatgggtgggagggcgggtactatccacaggtcaacc

SEQ ID NO: 17 - DNA, 83 bp, Artificial Sequence
  S1 Aptamer B-11A (S1_B11-A)
ccagtgtagactactcaatgcgtgtggtgaccttgaccttggattatgggtttgggcgggagggtactatccacaggtcaacc

SEQ ID NO: 18 - DNA, 82 bp, Artificial Sequence
  S1 Aptamer B12-B (S1_B12-B)
ccagtgtagactactcaatgctgtgttgacttgatcctgtggtatatgggtgggagggatgggtactatccacaggtcaacc

SEQ ID NO: 19 - DNA, 82 bp, Artificial Sequence
  S1 Aptamer C1-A (S1_C1-A)
ccagtgtagactactcaatgctgtgttgacttgatcctgtggtatttgggtgggaggggcgggtactatccacaggtcaacc

SEQ ID NO: 20 - DNA, 81 bp, Artificial Sequence
  S1 Aptamer C1-B (S1_C1-B)
ccagtgtagactactcaatgccgcttctcgaccgccccttgttttggttcttttgtccgtagtactatccacaggtcaacc

SEQ ID NO: 21 - DNA, 82 bp, Artificial Sequence
  S1 Aptamer C2 (S1_C2)
ccagtgtagactactcaatgcggctgtgtgacttgacctctggatatgggtgggagggatgggtactatccacaggtcaacc

SEQ ID NO: 22 - DNA, 81 bp, Artificial Sequence
  S1 Aptamer C3-A (S1_C3-A)
ccagtgtagactactcaatgctgtgttgacttgatcctgtggcatatgggtgggagggcgggtactatccacaggtcaacc

SEQ ID NO: 23 - DNA, 82 bp, Artificial Sequence
  S1 Aptamer C3-B (S1_C3-B)
ccagtgtagactactcaatgccgatgggtcgggggggtgggtaggcattgatcgctccttccgtactatccacaggtcaacc

SEQ ID NO: 24 - DNA, 81 bp, Artificial Sequence
  S1 Aptamer C4 (S1_C4)
ccagtgtagactactcaatgctactgattcatctgccgaatccaaacccgaccagtccacggtactatccacaggtcaacc

SEQ ID NO: 25 - DNA, 82 bp, Artificial Sequence
  S1 Aptamer C8 (S1_C8)
ccagtgtagactactcaatgccgatgggtcgggggggtgggtaggcattgatcattcctttcgtactatccacaggtcaacc

SEQ ID NO: 26 - DNA, 82 bp, Artificial Sequence
  S1 Aptamer C9 (S1_C9)
  misc_feature (44)..(44): n is a, c, g, or t
ccagtgtagactactcaatgcgtgggtaagggtgggtgggagcnttgataactcggaggagcgtactatccacaggtcaacc

SEQ ID NO: 27 - DNA, 81 bp, Artificial Sequence
  S1 Aptamer C10 (S1_C10)
ccagtgtagactactcaatgctgtgttgacttgatcctgcgggatatgggtggcattgagggtactatccacaggtcaacc

SEQ ID NO: 28 - DNA, 82 bp, Artificial Sequence
  S1 Aptamer C11 (S1_C11)
ccagtgtagactactcaatgccgtggactggtcgggtttggattcggcagatgaatccagtagtactatccacaggtcaacc

SEQ ID NO: 29 - DNA, 81 bp, Artificial Sequence
  S1 Aptamer C12 (S1_C12)
ccagtgtagactactcaatgcgtgggaagggtgggtgggagcattgataactcggaggagcgtactatccacaggtcaacc

SEQ ID NO: 30 - DNA, 81 bp, Artificial Sequence
  S1 Aptamer D1 (S1_D1)
ccagtgtagactactcaatgcattccttttgctacctatattccgtctccagcataatcccgtactatccacaggtcaacc

SEQ ID NO: 31 - DNA, 81 bp, Artificial Sequence
  S1 Aptamer D2 (S1_D2)
ccagtgtagactactcaatgctaggtccgtgggatgggtgggggggaggaccagccagcgagtactatccacaggtcaacc

SEQ ID NO: 32 - DNA, 81 bp, Artificial Sequence
  S1 Aptamer D4 (S1_D4)
ccagtgtagactactcaatgcggcttcgggagggggggcgggtaaaaagcccattgccctagtactatccacaggtcaacc

SEQ ID NO: 33 - DNA, 81 bp, Artificial Sequence
  S1 Aptamer D6 (S1_D6)
ccagtgtagactactcaatgctactgactcatctgccgaatccaaacccgaccagtccacggtactatccacaggtcaacc

SEQ ID NO: 34 - DNA, 81 bp, Artificial Sequence
  S1 Aptamer D8 (S1_D8)
cagtgtaagactactcaatgccgtggactggtcgggtttggattcggcagatgaatcagtagtactatccacaggtcaacc

SEQ ID NO: 35 - DNA, 81 bp, Artificial Sequence
  S1 Aptamer D10-A (S1_D10-A)
  misc_feature (47)..(47): n is a, c, g, or t
ccagtgtagactactcaatgcggctgtgtgacttgacctctggatangggagggagggcgggtactatccacaggtcaacc

SEQ ID NO: 36 - DNA, 81 bp, Artificial Sequence
  S1 Aptamer D11-B (S1_D11-B)
ccagtgtagactactcaatgccggccgggagggaggggtgggtaaagccgcgcaacatatagtactatccacaggtcaacc

SEQ ID NO: 37 - DNA, 82 bp, Artificial Sequence
  S1 Aptamer E2 (S1_E2)
ccagtgtagactactcaatgcgtgtggtgaccttgacctctggatatgggtgggaggggcgggtactatccacaggtcaacc

SEQ ID NO: 38 - DNA, 81 bp, Artificial Sequence
  S1 Aptamer E3 (S1_E3)
ccagtgtagactactcaatgccgatgggtcgggtgggtgggtaggcattgatcgctcctccgtactatccacaggtcaacc

SEQ ID NO: 39 - DNA, 81 bp, Artificial Sequence
  S1 Aptamer E5 (S1_E5)
ccagtgtagactactcaatgccacctccttttccgtttcttgtatcatgctactatcctgtgtactatccacaggtcaacc

SEQ ID NO: 40 - DNA, 85 bp, Artificial Sequence
  S1 Aptamer E6 (S1_E6)
ccagtgtagactactcaatgcccagtggcgagcgactttttatgtaagaacatcgattttccatggtactatccacaggtcaacc

SEQ ID NO: 41 - DNA, 81 bp, Artificial Sequence
  S1 Aptamer E9 (S1_E9)
ccagtgtagactactcaatgcggcttcgagagggggggcgggtaaaaagtccattgccctagtactatccacaggtcaacc

SEQ ID NO: 42 - DNA, 81 bp, Artificial Sequence
  S1 Aptamer E10 (S1_E10)
ccagtgtagactactcaatgctgtgttgacttgaccttggattatgggtttgggcgggagggtactatccacaggtcaacc

SEQ ID NO: 43 - DNA, 82 bp, Artificial Sequence
  S1 Aptamer F2 (S1_F2)
ccagtgtagactactcaatgctgcgttgacttgatcctgtggtatatgggtgggagggtcgggtactatccacaggtcaacc

SEQ ID NO: 44 - DNA, 81 bp, Artificial Sequence
  S1 Aptamer F3-A (S1_F3-A)
ccagtgtagactactcaatgcggtatgaatatgatcggccctatcccttcagctattccgggtactatccacaggtcaacc

SEQ ID NO: 45 - DNA, 81 bp, Artificial Sequence
  S1 Aptamer F3-B (S1_F3-B)
ccagtgtagactactcaatgcgtgggatgggtgggtgggagcattgataactcggaggagcgtactatccacaggtcaacc

SEQ ID NO: 46 - DNA, 83 bp, Artificial Sequence
  S1 Aptamer F5 (S1_F5)
ccagtgtagactactcaatgcgtgtggtaaccttgaccttggattatgggtttgggcgggcgggtactatccacaggtcaacc

SEQ ID NO: 47 - DNA, 91 bp, Artificial Sequence
  S1 Aptamer F7 (S1_F7)
ccagtgtagactactcaatgcccgccctccctcccatatccagaggtcaaggtcaccacacccataacgccgtactatccacaggtcaacc

SEQ ID NO: 48 - DNA, 82 bp, Artificial Sequence
  S1 Aptamer F11 (S1_F11)
ccagtgtagactactcaatgcgtgggaagggtgggcgggagcattgataacttcggaggagcgtactatccacaggtcaacc

SEQ ID NO: 49 - DNA, 81 bp, Artificial Sequence
  S1 Aptamer F12 (S1_F12)
ccagtgtagactactcaatgcgtgggaagggagggcgggagcattgataactcggaggagcgtactatccacaggtcaacc

SEQ ID NO: 50 - DNA, 82 bp, Artificial Sequence
  S1 Aptamer G4 (S1_G4)
ccagtgtagactactcaatgccgcattactagaatcctgtggtatatgggtgggaggggcgggtactatccacaggtcaacc

SEQ ID NO: 51 - DNA, 81 bp, Artificial Sequence
  S1 Aptamer G5 (S1_G5)
ccagtgtagactactcaatgcgtgtggtgaccttgacctttggatatggaagggagggtgggtactatccacaggtcaacc

SEQ ID NO: 52 - DNA, 82 bp, Artificial Sequence
  S1 Aptamer G6 (S1_G6)
ccagtgtagactactcaatgctgtgttgacttgatcctgtggtatatgggtgggaggggcgggtactatccacaggtcaacc

SEQ ID NO: 53 - DNA, 62 bp, Artificial Sequence
  S1 Aptamer H12 (S1_H12)
tgcatagtctcagctgccgtggacaatcgatgttcttggagggtactatccacaggtcaacc

SEQ ID NO: 54 - DNA, 81 bp, Artificial Sequence
  RBD Aptamer A1 (RBD_A1)
ccagtgtagactactcaatgctgtgttgacttgaccttggattatgggtttgggcgggcgggtactatccacaggtcaacc

SEQ ID NO: 55 - DNA, 81 bp, Artificial Sequence
  RBD Aptamer A2 (RBD_A2)
ccagtgtagactactcaatgcgtgggaagggtgggtgggagcattgataactcggaggagagtactatccacaggtcaacc

SEQ ID NO: 56 - DNA, 81 bp, Artificial Sequence
  RBD Aptamer A4 (RBD_A4)
ccagtgtagactactcaatgcgcattcgtgttcctttattttttgtttttccttcgctctcgtactatccacaggtcaacc

SEQ ID NO: 57 - DNA, 81 bp, Artificial Sequence; identical to SEQ ID NO: 45
  RBD Aptamer A5 (RBD_A5)

SEQ ID NO: 58 - DNA, 82 bp, Artificial Sequence
  RBD Aptamer A6 (RBD_A6)
ccagtgtagactactcaatgcggctgtgtggcttgacctctggatatgggtgggagggatgggtactatccacaggtcaacc

SEQ ID NO: 59 - DNA, 81 bp, Artificial Sequence; identical to SEQ ID NO: 32
  RBD Aptamer A7 (RBD_A7)

SEQ ID NO: 60 - DNA, 81 bp, Artificial Sequence; identical to SEQ ID NO: 29
  RBD Aptamer A8 (RBD_A8)

SEQ ID NO: 61 - DNA, 80 bp, Artificial Sequence
  RBD Aptamer A9 (RBD_A9)
ccagtgtagactactcaatgcggggaaaggagggcgggagcattgatagctcggaggagagtactatccacaggtcaacc

SEQ ID NO: 62 - DNA, 81 bp, Artificial Sequence; identical to SEQ ID NO: 14
  RBD Aptamer A10 (RBD_A10)

SEQ ID NO: 63 - DNA, 81 bp, Artificial Sequence
  RBD Aptamer A12-B (RBD_A12-B)
ccagtgtagactactcaatgccgatgggtcgggcgggtgggtaggcattgatcgctcctccgtactatccacaggtcaacc

SEQ ID NO: 64 - DNA, 80 bp, Artificial Sequence
  RBD Aptamer B1 (RBD_B1)
ccagtgtagactactcaatgcgtgggaagggtgggggggagcttgataactcgcaggagagtactatccacaggtcaacc

SEQ ID NO: 65 - DNA, 81 bp, Artificial Sequence; identical to SEQ ID NO: 4
  RBD Aptamer B2 (RBD_B2)

SEQ ID NO: 66 - DNA, 81 bp, Artificial Sequence; identical to SEQ ID NO: 6
  RBD Aptamer B3 (RBD_B3)

SEQ ID NO: 67 - DNA, 81 bp, Artificial Sequence; identical to SEQ ID NO: 38
  RBD Aptamer B4-A (RBD_B4-A)

SEQ ID NO: 68 - DNA, 81 bp, Artificial Sequence
  RBD Aptamer B4-5 (RBD_B4-B)
ccagtgtagactactcaatgcagggcgggggggagggtattgcattgcttaatcgagggtagtactatccacaggtcaacc

SEQ ID NO: 69 - DNA, 82 bp, Artificial Sequence; identical to SEQ ID NO: 50
  RBD Aptamer B6-B (RBD_B6-B)

SEQ ID NO: 70 - DNA, 80 bp, Artificial Sequence
  RBD Aptamer B10 (RBD_B10)
ccagtgtagactactcaatgcgtggtatattgatcctgtgcattgataatgcgaaggatggtactatccacaggtcaacc

SEQ ID NO: 71 - DNA, 84 bp, Artificial Sequence
  RBD Aptamer B12 (RBD_B12)
ccagtgtagactactcaatgccgtgtggtgaccttgatcctgtggtatatgggtgggagggagggtactatccacaggtcaacc

SEQ ID NO: 72 - DNA, 81 bp, Artificial Sequence
  RBD Aptamer C1 (RBD_C1)
ccagtgtagactactcaatgcgtgggaagggtgggcgggagcattgataactcggaggagagtactatccacaggtcaacc

SEQ ID NO: 73 - DNA, 81 bp, Artificial Sequence
  RBD Aptamer C7-A (RBD_C7-A)
ccagtgtagactactcaatgccgatgggtcgggtgggtgggtaggcattggtcgctcctccgtactatccacaggtcaacc

SEQ ID NO: 74 - DNA, 81 bp, Artificial Sequence
  RBD Aptamer C7-B (RBD_C7-B)
ccagtgtagactactcaatgcgtgtggtgaccttgacctctggatatgggtgggagggagggtactatccacaggtcaacc

SEQ ID NO: 75 - DNA, 81 bp, Artificial Sequence
  RBD Aptamer C8-A (RBD_C8-A)
ccagtgtagactactcaatgcgtgggaagggtgggtgggagcattgatagctcggaggagagtactatccacaggtcaacc

SEQ ID NO: 76 - DNA, 81 bp, Artificial Sequence
  RBD Aptamer C11-B (RBD_C11-B)
ccagtgtagactactcaatgcgttaggtgaccttgacctccggatatgggagggagggcgggtactatccacaggtcaacc

SEQ ID NO: 77 - DNA, 81 bp, Artificial Sequence
  RBD Aptamer D1 (RBD_D1)
ccagtgtagactactcaatgcacctagtgcccgctattcacaataattactactatctcgtgtactatccacaggtcaacc

SEQ ID NO: 78 - DNA, 85 bp, Artificial Sequence
  RBD Aptamer D2 (RBD_D2)
ccagtgtagactactcaatgctaggtgtggtgaccttgacctctggatacgggtgggaggggcgggtactatccacaggtcaacc

SEQ ID NO: 79 - DNA, 81 bp, Artificial Sequence
  RBD Aptamer D3 (RBD_D3)
ccagtgtagactactcaatgcgtgggaagggtgggcgggagcattgataactcggaggggcgtactatccacaggtcaacc

SEQ ID NO: 80 - DNA, 81 bp, Artificial Sequence
  RBD Aptamer D4 (RBD_D4)
ccagtgtagactactcaatgcggcctcgggagggggggcgggtaaaaggcccattgccctagtactatccacaggtcaacc

SEQ ID NO: 81 - DNA, 81 bp, Artificial Sequence
  RBD Aptamer D5 (RBD_D5)
ccagtgtagactactcaatgcgtgggatgggtgggtgggagcattgataactcggaggagagtactatccacaggtcaacc

SEQ ID NO: 82 - DNA, 86 bp, Artificial Sequence
  RBD Aptamer D7 (RBD_D7)
ccagtgtagactactcaatgccgtggtggtgaccttgatcctgtggtatatgggtgggaggggcgggtactatccacaggtcaacc

SEQ ID NO: 83 - DNA, 81 bp, Artificial Sequence
  RBD Aptamer D8 (RBD_D8)
ccagtgtagactactcaatgcagggtgggagggagggtattgcattgcctaatcggggagagtactatccacaggtcaacc

SEQ ID NO: 84 - DNA, 84 bp, Artificial Sequence
  RBD Aptamer D9 (RBD_D9)
ccagtgtagactactcaatgccgtgtggtgaccttgaccttggattatgggtttgggcgggagggtactatccacaggtcaacc

SEQ ID NO: 85 - DNA, 82 bp, Artificial Sequence
  RBD Aptamer D10 (RBD_D10)
ccagtgtagactactcaatgcagggtgggagggaagggagtgcattgcctaatcggggcagagtactatccacaggtcaacc

SEQ ID NO: 86 - DNA, 82 bp, Artificial Sequence; identical to SEQ ID NO: 18
  RBD Aptamer D12-A (RBD_D12-A)

SEQ ID NO: 87 - DNA, 84 bp, Artificial Sequence
  RBD Aptamer D12-B (RBD_D12-B)
ccagtgtagactactcaatgccgtgtggtgaccttgatcctgtggtatatgggtgggggggagggtactatccacaggtcaacc

SEQ ID NO: 88 - DNA, 81 bp, Artificial Sequence
  RBD Aptamer E1 (RBD_E1)
ccagtgtagactactcaatgcggcttcgggagggggggcgggtaaaaagtccattgccctagtactatccacaggtcaacc

SEQ ID NO: 89 - DNA, 81 bp, Artificial Sequence
  RBD Aptamer E3 (RBD_E3)
ccagtgtagactactcaatgcgtggggggggtgggtgggagcattgataagtcggaggagcgtactatccacaggtcaacc

SEQ ID NO: 90 - DNA, 81 bp, Artificial Sequence
  RBD Aptamer E5 (RBD_E5)
ccagtgtagactactcaatgcgtgggatgggtgggtgggagcattgatagctcggaggagcgtactatccacaggtcaacc

SEQ ID NO: 91 - DNA, 81 bp, Artificial Sequence
  RBD Aptamer E6 (RBD_E6)
ccagtgtagactactcaatgcgtgggatgggtgggggggagcattgataactcgggggagcgtactatccacaggtcaacc

SEQ ID NO: 92 - DNA, 81 bp, Artificial Sequence
  RBD Aptamer E8-B (RBD_E8-B)
ccagtgtagactactcaatgccgatgggttgggggggtgggtaggcattgatcgctcctccgtactatccacaggtcaacc

SEQ ID NO: 93 - DNA, 81 bp, Artificial Sequence
  RBD Aptamer E11 (RBD_E11)
ccagtgtagactactcaatgcagggtgggagggagggtattgcattgcctaatcgaggggagtactatccacaggtcaacc

SEQ ID NO: 94 - DNA, 85 bp, Artificial Sequence
  RBD Aptamer F5 (RBD_F5)
ccagtgtagactactcaatgccgtgtggtgaccttgaccctgtggtatatgggtgggaggggtgggtactatccacaggtcaacc

SEQ ID NO: 95 - DNA, 82 bp, Artificial Sequence
  RBD Aptamer F12 (RBD_F12)
ccagtgtagactactcaatgctagggcgggagggagggtatttagcatttacacgttagatagtactatccacaggtcaacc

SEQ ID NO: 96 - DNA, 78 bp, Artificial Sequence
  RBD Aptamer G1-A (RBD_G1-A)
ccagtgtagactactcaatgcgtgggaagggtgggtgggagcattgataaccctgatagtactatccacaggtcaacc

SEQ ID NO: 97 - DNA, 81 bp, Artificial Sequence
  RBD Aptamer G1-B (RBD_G1-B)
ccagtgtagactactcaatgccgatgggttggggggtgggtaggcattgatcgctcctttcgtactatccacaggtcaacc

SEQ ID NO: 98 - DNA, 83 bp, Artificial Sequence
  RBD Aptamer G2 (RBD_G2)
ccagtgtagactactcaatgcgtgtggtgaccttgaccttggattatgggtttgggcgggcgggtactatccacaggtcaacc

SEQ ID NO: 99 - DNA, 81 bp, Artificial Sequence
  RBD Aptamer G9-A (RBD_G9-A)
ccagtgtagactactcaatgcgagggatgggtgggtgggagcattgataactcggaggagcgtactatccacaggtcaacc

SEQ ID NO: 100 - DNA, 81 bp, Artificial Sequence
  RBD Aptamer G9-B (RBD_G9-B)
ccagtgtagactactcaatgctgtgttgacttgaccttggattatgggtttgggcgggtgggtactatccacaggtcaacc

SEQ ID NO: 101 - DNA, 81 bp, Artificial Sequence
  RBD Aptamer G10-B (RBD_G10-B)
ccagtgtagactactcaatgcggcttcgggagggggggcgggtaaaaagcccattaccctagtactatccacaggtcaacc

SEQ ID NO: 102 - DNA, 81 bp, Artificial Sequence
  RBD Aptamer G11 (RBD_G11)
ccagtgtagactactcaatgcgtgggaagggtgggtgggagcattgataactcgtaggagagtactatccacaggtcaacc

SEQ ID NO: 103 - DNA, 81 bp, Artificial Sequence
  RBD Aptamer G12-B (RBD_G12-B)
ccagtgtagactactcaatgcggctgtgtgacttgacctctggatatgggtgggagggggggtactatccacaggtcaacc

SEQ ID NO: 104 - DNA, 81 bp, Artificial Sequence
  RBD Aptamer H1 (RBD_H1)
ccagtgtagactactcaatgcagggtgggagggagggtattgcattgcctaatcgaggagagtactatccacaggtcaacc

SEQ ID NO: 105 - DNA, 80 bp, Artificial Sequence
  RBD Aptamer H2 (RBD_H2)
ccagtgtagactactcaatgcggggggggggggggggggaggattgataacggggaggagggtactataccgggtcccat

SEQ ID NO: 106 - DNA, 79 bp, Artificial Sequence
  RBD Aptamer H4 (RBD_H4)
ccaggtagactactcaatgcgtgggaagggtgggcgggagcatgataactcggaggagagtactatccacaggtcacgc

SEQ ID NO: 107 - DNA, 80 bp, Artificial Sequence
  RBD Aptamer H5 (RBD_H5)
ccagtgtagactactcaatgcgtgtggtgaccttgaaatgatgatatgggtgggaggggcgtactatccagaggtcaacc

SEQ ID NO: 108 - DNA, 82 bp, Artificial Sequence; identical to SEQ ID NO: 5
  RBD Aptamer H6-A (RBD_H6-A)

SEQ ID NO: 109 - DNA, 82 bp, Artificial Sequence
  RBD Aptamer H6-B (RBD_H6-B)
ccagtgtagactactcaatgctgtgttgacttgatcctgtggtatatgggtgggaaggatgggtactatccacaggtcaacc

SEQ ID NO: 110 - DNA, 85 bp, Artificial Sequence
  RBD Aptamer H7-A (RBD_H7-A)
ccagtgtagactactcaatgccgtgtggtgaccttgactctgtggtatatgggtgggaggggcgggtactatccacaggtcaacc

SEQ ID NO: 111 - DNA, 82 bp, Artificial Sequence; identical to SEQ ID NO: 21
  RBD Aptamer H7-B (RBD_H7-B)

SEQ ID NO: 112 - DNA, 83 bp, Artificial Sequence; identical to SEQ ID NO: 17
  RBD Aptamer H8 (RBD_H8)

SEQ ID NO: 113 - DNA, 81 bp, Artificial Sequence
  RBD Aptamer H9 (RBD_H9)
ccagtgtagactactcaatgcccgtattctctagatttatgcttatccatcttcttcctaagtactatccacaggtcaacc

SEQ ID NO: 114 - DNA, 20 bp, Artificial Sequence
  first primer region (P1)
ccagtgtagactactcaatg

SEQ ID NO: 115 - DNA, 20 bp, Artificial Sequence
  second primer region (P2)
gtactatccacaggtcaacc

SEQ ID NO: 116 - DNA, 20 bp, Artificial Sequence
  reverse second primer region (P2)
ggttgacctgtggatagtac

SEQ ID NO: 117 - protein, 1273 aa, Artificial Sequence
  SARS-CoV-2 spike protein amino acid sequence (P0DTC2, UniProtKB version 1)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 118 - protein, 670 aa, Artificial Sequence
  SARS-CoV-2 spike protein S1 subunit amino acid sequence (P0DTC2, Val16- Arg685, UniProtKB version 1)
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 119 - protein, 588 aa, Artificial Sequence
  SARS-CoV-2 spike protein S2 subunit amino acid sequence (P0DTC2, Ser686- Thr1273, UniProtKB version 1)
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 120 - protein, 223 aa, Artificial Sequence
  SARS-CoV-2 spike protein Receptor-binding domain (RBD) amino acid sequence (P0DTC2, Arg319- Phe541, UniProtKB version 1)
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 121 - protein, 1255 aa, Artificial Sequence
  SARS-CoV spike protein full sequence amino acid sequence (P59594, UniProtKB version 1)
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 122 - protein, 667 aa, Artificial Sequence
  SARS-CoV spike protein S1 subunit amino acid sequence (P59594, Met1-Arg667, UniProtKB version 1)
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLR

SEQ ID NO: 123 - protein, 588 aa, Artificial Sequence
  SARS-CoV spike protein S2 subunit amino acid sequence (P59594, Ser668- Thr1255, UniProtKB version 1)
STSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 124 - protein, 222 aa, Artificial Sequence
  SARS-CoV spike protein Receptor-binding domain (RBD) amino acid sequence (P59594, Arg306- Phe527, UniProtKB version 1)
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNF

SEQ ID NO: 125 - protein, 1353 aa, Artificial Sequence
  MERS-CoV spike protein full sequence amino acid sequence (K9N5Q8, UniProtKB version 1)
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRFLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 126 - protein, 725 aa, Artificial Sequence
  MERS-CoV spike protein S1 subunit amino acid sequence (K9N5Q8, Met1-Glu725, UniProtKB version 1)
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSHTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVE

SEQ ID NO: 127 - protein, 602 aa, Artificial Sequence
  MERS-CoV spike protein S2 subunit amino acid sequence (K9N5Q8, Ser752-His1353, UniProtKB version 1)
SVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 128 - protein, 240 aa, Artificial Sequence
  MERS-CoV spike protein Receptor-binding domain (RBD) amino acid sequence (K9N5Q8, Glu367- Tyr606, UniProtKB version 1)
EAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRFLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEY

SEQ ID NO: 129 - DNA, 40 bp, Artificial Sequence
  nucleic acid fragment (F9) of S1 Aptamer A3 (S1_A3)
ccagtgtagactactcaatgcgtgggaagggtgggtggga

SEQ ID NO: 130 - DNA, 48 bp, Artificial Sequence
  nucleic acid fragment (F10) of S1 Aptamer A3 (S1_A3)
ccagtgtagactactcaatgcgtgggaagggtgggtgggagcattgat

SEQ ID NO: 131 - DNA, 53 bp, Artificial Sequence
  nucleic acid fragment (F11) of S1 Aptamer A3 (S1_A3)
ccagtgtagactactcaatgcgtgggaagggtgggtgggagcattgataactc

SEQ ID NO: 132 - DNA, 64 bp, Artificial Sequence
  nucleic acid fragment (F12) of S1 Aptamer A3 (S1_A3)
ccagtgtagactactcaatgcgtgggaagggtgggtgggagcattgataactcggaggagtgta

SEQ ID NO: 133 - DNA, 25 bp, Artificial Sequence
  nucleic acid fragment (F17) of S1 Aptamer A3 (S1_A3)
aatgcgtgggaagggtgggtgggag

SEQ ID NO: 134 - DNA, 33 bp, Artificial Sequence
  nucleic acid fragment (F18) of S1 Aptamer A3 (S1_A3)
aatgcgtgggaagggtgggtgggagcattgata

SEQ ID NO: 135 - DNA, 40 bp, Artificial Sequence
  nucleic acid fragment (F2) of S1 Aptamer A8 (S1_A8)
gtatatgggtgggagggtcgggtactatccacaggtcaac

SEQ ID NO: 136 - DNA, 64 bp, Artificial Sequence
  nucleic acid fragment (F12) of S1 Aptamer A8 (S1_A8)
ccagtgtagactactcaatgctgtgttgacttgatcctgtggtatatgggtgggagggtcgggt

SEQ ID NO: 137 - DNA, 65 bp, Artificial Sequence
  nucleic acid fragment (F13) of S1 Aptamer A8 (S1_A8)
aatgctgtgttgacttgatcctgtggtatatgggtgggagggtcgggtactatccacaggtcaac

SEQ ID NO: 138 - DNA, 53 bp, Artificial Sequence
  nucleic acid fragment (F14) of S1 Aptamer A8 (S1_A8)
acttgatcctgtggtatatgggtgggagggtcgggtactatccacaggtcaac

SEQ ID NO: 139 - DNA, 36 bp, Artificial Sequence
  nucleic acid fragment (F20) of S1 Aptamer A8 (S1_A8)
acttgatcctgtggtatatgggtgggagggtcgggt

SEQ ID NO: 140 - DNA, 32 bp, Artificial Sequence
  nucleic acid fragment (F21) of S1 Aptamer A8 (S1_A8)
gatcctgtggtatatgggtgggagggtcgggt

SEQ ID NO: 141 - DNA, 24 bp, Artificial Sequence
  nucleic acid fragment (F22) of S1 Aptamer A8 (S1_A8)
ggtatatgggtgggagggtcgggt

SEQ ID NO: 142 - DNA, 64 bp, Artificial Sequence
  nucleic acid fragment (F12) of S1 Aptamer A6 (S1_A6)
ccagtgtagactactcaatgctgttatgacttgaccttgggttatgggtttgggcgggagggta

SEQ ID NO: 143 - DNA, 65 bp, Artificial Sequence
  nucleic acid fragment (F13) of S1 Aptamer A6 (S1_A6)
aatgctgttatgacttgaccttgggttatgggtttgggcgggagggtactatccacaggtcaacc

SEQ ID NO: 144 - DNA, 53 bp, Artificial Sequence
  nucleic acid fragment (F14) of S1 Aptamer A6 (S1_A6)
acttgaccttgggttatgggtttgggcgggagggtactatccacaggtcaacc

SEQ ID NO: 145 - DNA, 81 bp, Artificial Sequence
  S2 Aptamer A1 (S2_A1)
ccagtgtagactactcaatgcgtgggaagggtgggtgggagcattgataactcgtgggagcgtactatccacaggtcaacc

SEQ ID NO: 146 - DNA, 82 bp, Artificial Sequence; identical to SEQ ID NO: 5
  S2 Aptamer A2 (S2_A2)

SEQ ID NO: 147 - DNA, 81 bp, Artificial Sequence; identical to SEQ ID NO: 32
  S2 Aptamer A6 (S2_A6)

SEQ ID NO: 148 - DNA, 82 bp, Artificial Sequence
  S2 Aptamer A10 (S2_A10)
ccagtgtagactactcaatgccggtgggtcgggggggtgggtaggcattgatcgctccttccgtactatccacaggtcaacc

SEQ ID NO: 149 - DNA, 81 bp, Artificial Sequence; identical to SEQ ID NO: 38
  S2 Aptamer A12 (S2_A12)

SEQ ID NO: 150 - DNA, 81 bp, Artificial Sequence
  S2 Aptamer B1 (S2_B1)
ccagtgtagactactcaatgccgtggactggtcgggtttggattcggcagatgaatcagtagtactatccacaggtcaacc

SEQ ID NO: 151 - DNA, 85 bp, Artificial Sequence; identical to SEQ ID NO: 11
  S2 Aptamer B2 (S2_B2)

SEQ ID NO: 152 - DNA, 82 bp, Artificial Sequence
  S2 Aptamer B4 (S2_B4)
ccagtgtagactactcaatgccgatgggtcgggggggtgggtaggcattgatccttcctttcgtactatccacaggtcaacc

SEQ ID NO: 153 - DNA, 81 bp, Artificial Sequence
  S2 Aptamer B6 (S2_B6)
ccagtgtagactactcaatgccgtggactggtcgggtttggattcggcaggtgaatcagtagtactatccacaggtcaacc

SEQ ID NO: 154 - DNA, 82 bp, Artificial Sequence
  S2 Aptamer B11 (S2_B11)
ccagtgtagactactcaatgcattagacccctaatatacgatttccacccaatttttcccacgtactatccacaggtcaacc

SEQ ID NO: 155 - DNA, 81 bp, Artificial Sequence; identical to SEQ ID NO: 24
  S2 Aptamer B12 (S2_B12)

SEQ ID NO: 156 - DNA, 81 bp, Artificial Sequence
  S2 Aptamer C6 (S2_C6)
ccagtgtagactactcaatgccgtggactggtcgggtttggattccccacatgaatcagtagtactatccacaggtcaacc

SEQ ID NO: 157 - DNA, 84 bp, Artificial Sequence
  S2 Aptamer C9 (S2_C9)
ccagtgtagactactcaatgctgctgtgtcgacttgatcctgtggtatatgggtgggagggagggtactatccacaggtcaacc

SEQ ID NO: 158 - DNA, 81 bp, Artificial Sequence
  S2 Aptamer D4 (S2_D4)
ccagtgtagactactcaatgctgtccgggtgggagggggggtttgacagcatctgcaggtagtactatccacaggtcaacc

SEQ ID NO: 159 - DNA, 72 bp, Artificial Sequence
  S2 Aptamer D5 (S2_D5)
actcaatgctaggtgtggtgaccttgacctctggatatgggagggagggtgggtactatccacaggtcaacc

SEQ ID NO: 160 - DNA, 81 bp, Artificial Sequence; identical to SEQ ID NO: 54
  S2 Aptamer D7 (S2_D7)

SEQ ID NO: 161 - DNA, 81 bp, Artificial Sequence; identical to SEQ ID NO: 29
  S2 Aptamer D8 (S2_D8)

SEQ ID NO: 162 - DNA, 81 bp, Artificial Sequence
  S2 Aptamer D10 (S2_D10)
ccagtgtagactactcaatgcggcttcgggagggggggcaggtaaaaagcccattgctctagtactatccacaggtcaacc

SEQ ID NO: 163 - DNA, 85 bp, Artificial Sequence
  S2 Aptamer D11 (S2_D11)
ccagtgtagactactcaatgccgtgtggtgaccttgatcctgtggtatatgggtgggagggatgggtactatccacaggtcaacc

SEQ ID NO: 164 - DNA, 81 bp, Artificial Sequence
  S2 Aptamer E2 (S2_E2)
ccagtgtagactactcaatgcctccggaagttctgtatcattaaactccatcctcccacgcgtactatccacaggtcaacc

SEQ ID NO: 165 - DNA, 81 bp, Artificial Sequence
  S2 Aptamer E3 (S2_E3)
ccagtgtagactactcaatgccgtggactggtcgggtttggattcggcagatgaattagtagtactatccacaggtcaacc

SEQ ID NO: 166 - DNA, 82 bp, Artificial Sequence
  S2 Aptamer E5 (S2_E5)
ccagtgtagactactcaatgccgatgggtcgggggggtgggtaggcattgatcattcccttcgtactatccacaggtcaacc

SEQ ID NO: 167 - DNA, 84 bp, Artificial Sequence
  S2 Aptamer E8 (S2_E8)
ccagtgtagactactcaatgctaggtgtggtgaccttgacctctggatatgggagggagggcgggtactatccacaggtcaacc

SEQ ID NO: 168 - DNA, 81 bp, Artificial Sequence
  S2 Aptamer E9 (S2_E9)
ccagtgtagactactcaatgccgtggactggtcgggtttggattcggctgatgaaacagtagtactatccacaggtcaacc

SEQ ID NO: 169 - DNA, 81 bp, Artificial Sequence
  S2 Aptamer E10 (S2_E10)
ccagtgtagactactcaatgctcatatattcacctctctccccaaccctaatccacatcacgtactatccacaggtcaacc

SEQ ID NO: 170 - DNA, 82 bp, Artificial Sequence
  S2 Aptamer F10 (S2_F10)
ccagtgtagactactcaatgccgtgtggtgaccttgacctctggatatgggtgggagggagggtactatccacaggtcaacc

SEQ ID NO: 171 - DNA, 82 bp, Artificial Sequence; identical to SEQ ID NO: 21
  S2 Aptamer G1 (S2_G1)

SEQ ID NO: 172 - DNA, 81 bp, Artificial Sequence
  S2 Aptamer G7 (S2_G7)
ccagtgtagactactcaatgccgtggactggttgggtttggattcggcaggtgaatcagtagtactatccacaggtcaacc

SEQ ID NO: 173 - DNA, 81 bp, Artificial Sequence
  S2 Aptamer G9 (S2_G9)
ccagtgtagactactcaatgcgtgtggtgaccttgacctctggatatgggtgggagggggggtactatccacaggtcaacc

SEQ ID NO: 174 - DNA, 82 bp, Artificial Sequence; identical to SEQ ID NO: 23
  S2 Aptamer H3 (S2_H3)

SEQ ID NO: 175 - DNA, 81 bp, Artificial Sequence
  S2 Aptamer H6 (S2_H6)
ccagtgtagactactcaatgccgtgggctggtcgggtttggattcggcagatgaatcagtagtactatccacaggtcaacc

SEQ ID NO: 176 - DNA, 81 bp, Artificial Sequence; identical to SEQ ID NO: 54
  S2 Aptamer H9 (S2_H9)

SEQ ID NO: 177 - DNA, 25 bp, Artificial Sequence
  nucleic acid fragment (F17) of S2 Aptamer A2 (S2_A2)
aatgccgatgggtcgggggggtggg

SEQ ID NO: 178 - DNA, 33 bp, Artificial Sequence
  nucleic acid fragment (F18) of S2 Aptamer A2 (S2_A2)
aatgccgatgggtcgggggggtgggtaggcatt

SEQ ID NO: 179 - DNA, 64 bp, Artificial Sequence
  nucleic acid fragment (F12) of S2 Aptamer A2 (S2_A2)
ccagtgtagactactcaatgccgatgggtcgggggggtgggtaggcattgatcgctcctttcgt

SEQ ID NO: 180 - DNA, 53 bp, Artificial Sequence
  nucleic acid fragment (F11) of S2 Aptamer A2 (S2_A2)
ccagtgtagactactcaatgccgatgggtcgggggggtgggtaggcattgatc

SEQ ID NO: 181 - DNA, 41 bp, Artificial Sequence
  nucleic acid fragment (F1) of S2 Aptamer A2 (S2_A2)
ccagtgtagactactcaatgccgatgggtcgggggggtggg

SEQ ID NO: 182 - DNA, 38 bp, Artificial Sequence
  nucleic acid fragment (F19) of S2 Aptamer A2 (S2_A2)
aatgccgatgggtcgggggggtgggtaggcattgatcg

SEQ ID NO: 183 - DNA, 33 bp, Artificial Sequence
  nucleic acid fragment (F18) of S2 Aptamer B1 (S2_B1)
aatgccgtggactggtcgggtttggattcggca

SEQ ID NO: 184 - DNA, 38 bp, Artificial Sequence
  nucleic acid fragment (F19) of S2 Aptamer B1 (S2_B1)
aatgccgtggactggtcgggtttggattcggcagatga

SEQ ID NO: 185 - DNA, 48 bp, Artificial Sequence
  nucleic acid fragment (F23) of S2 Aptamer B1 (S2_B1)
aatgccgtggactggtcgggtttggattcggcagatgaatcagtagta

SEQ ID NO: 186 - DNA, 48 bp, Artificial Sequence
  nucleic acid fragment (F10) of S2 Aptamer B1 (S2_B1)
ccagtgtagactactcaatgccgtggactggtcgggtttggattcggc

SEQ ID NO: 187 - DNA, 53 bp, Artificial Sequence
  nucleic acid fragment (F11) of S2 Aptamer B1 (S2_B1)
ccagtgtagactactcaatgccgtggactggtcgggtttggattcggcagatg

SEQ ID NO: 188 - DNA, 64 bp, Artificial Sequence
  nucleic acid fragment (F12) of S2 Aptamer B1 (S2_B1)
ccagtgtagactactcaatgccgtggactggtcgggtttggattcggcagatgaatcagtagta

SEQ ID NO: 189 - DNA, 37 bp, Artificial Sequence
  nucleic acid fragment (F20) of S2 Aptamer G1 (S2_G1)
gtgacttgacctctggatatgggtgggagggatgggt

SEQ ID NO: 190 - DNA, 32 bp, Artificial Sequence
  nucleic acid fragment (F21) of S2 Aptamer G1 (S2_G1)
ttgacctctggatatgggtgggagggatgggt

SEQ ID NO: 191 - DNA, 24 bp, Artificial Sequence
  nucleic acid fragment (F22) of S2 Aptamer G1 (S2_G1)
tggatatgggtgggagggatgggt

SEQ ID NO: 192 - DNA, 65 bp, Artificial Sequence
  nucleic acid fragment (F13) of S2 Aptamer G1 (S2_G1)
aatgcggctgtgtgacttgacctctggatatgggtgggagggatgggtactatccacaggtcaac

SEQ ID NO: 193 - DNA, 30 bp, artificial sequence
  exemplary sequence used in the Lateral Flow Device ′Control line′ to capture fragment (F21) of S2 Aptamer G1 (S2_G1)
acccatccctcccacccatatccagaggtc